SEQUENCE LISTING (610 sequences)

SEQ ID NO: 1 - RNA, 22 nt, Homo sapiens
ugagguaguagguuguauaguu

SEQ ID NO: 2 - RNA, 21 nt, Homo sapiens
cuauacaaucuacugucuuuc

SEQ ID NO: 3 - RNA, 22 nt, Homo sapiens
ugagguaguagguugugugguu

SEQ ID NO: 4 - RNA, 22 nt, Homo sapiens
cuauacaaccuacugccuuccc

SEQ ID NO: 5 - RNA, 22 nt, Homo sapiens
ugagguaguagguuguaugguu

SEQ ID NO: 6 - RNA, 22 nt, Homo sapiens
uagaguuacacccugggaguua

SEQ ID NO: 7 - RNA, 22 nt, Homo sapiens
agagguaguagguugcauaguu

SEQ ID NO: 8 - RNA, 22 nt, Homo sapiens
cuauacgaccugcugccuuucu

SEQ ID NO: 9 - RNA, 22 nt, Homo sapiens
ugagguaggagguuguauaguu

SEQ ID NO: 10 - RNA, 22 nt, Homo sapiens
cuauacggccuccuagcuuucc

SEQ ID NO: 11 - RNA, 22 nt, Homo sapiens
ugagguaguagauuguauaguu

SEQ ID NO: 12 - RNA, 22 nt, Homo sapiens
ugagguaguaguuuguacaguu

SEQ ID NO: 13 - RNA, 21 nt, Homo sapiens
cuguacaggccacugccuugc

SEQ ID NO: 14 - RNA, 23 nt, Homo sapiens
ucuuugguuaucuagcuguauga

SEQ ID NO: 15 - RNA, 22 nt, Homo sapiens
auaaagcuagauaaccgaaagu

SEQ ID NO: 16 - RNA, 23 nt, Homo sapiens
uacccuguagauccgaauuugug

SEQ ID NO: 17 - RNA, 22 nt, Homo sapiens
caaauucguaucuaggggaaua

SEQ ID NO: 18 - RNA, 23 nt, Homo sapiens
uacccuguagaaccgaauuugug

SEQ ID NO: 19 - RNA, 22 nt, Homo sapiens
acagauucgauucuaggggaau

SEQ ID NO: 20 - RNA, 22 nt, Homo sapiens
uagcagcacauaaugguuugug

SEQ ID NO: 21 - RNA, 22 nt, Homo sapiens
caggccauauugugcugccuca

SEQ ID NO: 22 - RNA, 22 nt, Homo sapiens
uagcagcacaucaugguuuaca

SEQ ID NO: 23 - RNA, 22 nt, Homo sapiens
cgaaucauuauuugcugcucua

SEQ ID NO: 24 - RNA, 22 nt, Homo sapiens
uagcagcacguaaauauuggcg

SEQ ID NO: 25 - RNA, 23 nt, Homo sapiens
caaagugcuuacagugcagguag

SEQ ID NO: 26 - RNA, 22 nt, Homo sapiens
acugcagugaaggcacuuguag

SEQ ID NO: 27 - RNA, 23 nt, Homo sapiens
uaaggugcaucuagugcagauag

SEQ ID NO: 28 - RNA, 23 nt, Homo sapiens
acugcccuaagugcuccuucugg

SEQ ID NO: 29 - RNA, 22 nt, Homo sapiens
aguuuugcauaguugcacuaca

SEQ ID NO: 30 - RNA, 23 nt, Homo sapiens
ugugcaaaucuaugcaaaacuga

SEQ ID NO: 31 - RNA, 23 nt, Homo sapiens
ugugcaaauccaugcaaaacuga

SEQ ID NO: 32 - RNA, 23 nt, Homo sapiens
uaaagugcuuauagugcagguag

SEQ ID NO: 33 - RNA, 22 nt, Homo sapiens
acugcauuaugagcacuuaaag

SEQ ID NO: 34 - RNA, 23 nt, Homo sapiens
caaagugcucauagugcagguag

SEQ ID NO: 35 - RNA, 22 nt, Homo sapiens
acuguaguaugggcacuuccag

SEQ ID NO: 36 - RNA, 22 nt, Homo sapiens
uagcuuaucagacugauguuga

SEQ ID NO: 37 - RNA, 21 nt, Homo sapiens
caacaccagucgaugggcugu

SEQ ID NO: 38 - RNA, 22 nt, Homo sapiens
aguucuucaguggcaagcuuua

SEQ ID NO: 39 - RNA, 22 nt, Homo sapiens
aagcugccaguugaagaacugu

SEQ ID NO: 40 - RNA, 22 nt, Homo sapiens
uggguuccuggcaugcugauuu

SEQ ID NO: 41 - RNA, 21 nt, Homo sapiens
aucacauugccagggauuacc

SEQ ID NO: 42 - RNA, 22 nt, Homo sapiens
uggcucaguucagcaggaacag

SEQ ID NO: 43 - RNA, 22 nt, Homo sapiens
cauugcacuugucucggucuga

SEQ ID NO: 44 - RNA, 22 nt, Homo sapiens
uucaaguaauccaggauaggcu

SEQ ID NO: 45 - RNA, 21 nt, Homo sapiens
uucaaguaauucaggauaggu

SEQ ID NO: 46 - RNA, 22 nt, Homo sapiens
ccuguucuccauuacuuggcuc

SEQ ID NO: 47 - RNA, 22 nt, Homo sapiens
agggcuuagcugcuugugagca

SEQ ID NO: 48 - RNA, 21 nt, Homo sapiens
uucacaguggcuaaguuccgc

SEQ ID NO: 49 - RNA, 22 nt, Homo sapiens
agagcuuagcugauuggugaac

SEQ ID NO: 50 - RNA, 22 nt, Homo sapiens
cacuagauugugagcuccugga

SEQ ID NO: 51 - RNA, 22 nt, Homo sapiens
aaggagcucacagucuauugag

SEQ ID NO: 52 - RNA, 22 nt, Homo sapiens
uagcaccaucugaaaucgguua

SEQ ID NO: 53 - RNA, 22 nt, Homo sapiens
ugaccgauuucuccugguguuc

SEQ ID NO: 54 - RNA, 22 nt, Homo sapiens
uagcaccauuugaaaucgguua

SEQ ID NO: 55 - RNA, 22 nt, Homo sapiens
uguaaacauccucgacuggaag

SEQ ID NO: 56 - RNA, 22 nt, Homo sapiens
cuuucagucggauguuugcagc

SEQ ID NO: 57 - RNA, 22 nt, Homo sapiens
uguaaacauccuacacucagcu

SEQ ID NO: 58 - RNA, 22 nt, Homo sapiens
cugggagguggauguuuacuuc

SEQ ID NO: 59 - RNA, 23 nt, Homo sapiens
uguaaacauccuacacucucagc

SEQ ID NO: 60 - RNA, 22 nt, Homo sapiens
uguaaacauccccgacuggaag

SEQ ID NO: 61 - RNA, 22 nt, Homo sapiens
cuuucagucagauguuugcugc

SEQ ID NO: 62 - RNA, 22 nt, Homo sapiens
uguaaacauccuugacuggaag

SEQ ID NO: 63 - RNA, 22 nt, Homo sapiens
cuuucagucggauguuuacagc

SEQ ID NO: 64 - RNA, 22 nt, Homo sapiens
uauugcacauuacuaaguugca

SEQ ID NO: 65 - RNA, 22 nt, Homo sapiens
caauuuagugugugugauauuu

SEQ ID NO: 66 - RNA, 22 nt, Homo sapiens
uggcagugucuuagcugguugu

SEQ ID NO: 67 - RNA, 22 nt, Homo sapiens
caaucagcaaguauacugcccu

SEQ ID NO: 68 - RNA, 23 nt, Rattus norvegicus
aggcaguguaguuagcugauugc

SEQ ID NO: 69 - RNA, 22 nt, Rattus norvegicus
aaucacuaaccacacagccagg

SEQ ID NO: 70 - RNA, 22 nt, Homo sapiens
uauugcacuugucccggccugu

SEQ ID NO: 71 - RNA, 23 nt, Homo sapiens
caaagugcuguucgugcagguag

SEQ ID NO: 72 - RNA, 22 nt, Homo sapiens
acugcugagcuagcacuucccg

SEQ ID NO: 73 - RNA, 22 nt, Homo sapiens
ugagguaguaaguuguauuguu

SEQ ID NO: 74 - RNA, 22 nt, Homo sapiens
aacccguagauccgaucuugug

SEQ ID NO: 75 - RNA, 22 nt, Homo sapiens
cacccguagaaccgaccuugcg

SEQ ID NO: 76 - RNA, 22 nt, Homo sapiens
caagcucgugucuguggguccg

SEQ ID NO: 77 - RNA, 22 nt, Homo sapiens
aacccguagauccgaacuugug

SEQ ID NO: 78 - RNA, 22 nt, Homo sapiens
caagcuuguaucuauagguaug

SEQ ID NO: 79 - RNA, 22 nt, Homo sapiens
caguuaucacagugcugaugcu

SEQ ID NO: 80 - RNA, 21 nt, Homo sapiens
uacaguacugugauaacugaa

SEQ ID NO: 81 - RNA, 23 nt, Homo sapiens
agcagcauuguacagggcuauga

SEQ ID NO: 82 - RNA, 23 nt, Homo sapiens
aaaagugcuuacagugcagguag

SEQ ID NO: 83 - RNA, 22 nt, Homo sapiens
cugcaauguaagcacuucuuac

SEQ ID NO: 84 - RNA, 21 nt, Homo sapiens
uaaagugcugacagugcagau

SEQ ID NO: 85 - RNA, 22 nt, Homo sapiens
ccgcacuguggguacuugcugc

SEQ ID NO: 86 - RNA, 23 nt, Homo sapiens
agcagcauuguacagggcuauca

SEQ ID NO: 87 - RNA, 22 nt, Homo sapiens
acaggugagguucuugggagcc

SEQ ID NO: 88 - RNA, 24 nt, Homo sapiens
ucccugagacccuuuaaccuguga

SEQ ID NO: 89 - RNA, 22 nt, Homo sapiens
ucccugagacccuaacuuguga

SEQ ID NO: 90 - RNA, 21 nt, Homo sapiens
cauuauuacuuuugguacgcg

SEQ ID NO: 91 - RNA, 22 nt, Homo sapiens
ucguaccgugaguaauaaugcg

SEQ ID NO: 92 - RNA, 21 nt, Homo sapiens
ucacagugaaccggucucuuu

SEQ ID NO: 93 - RNA, 22 nt, Homo sapiens
uucacauugugcuacugucugc

SEQ ID NO: 94 - RNA, 22 nt, Homo sapiens
cagugcaauguuaaaagggcau

SEQ ID NO: 95 - RNA, 21 nt, Homo sapiens
acucuuucccuguugcacuac

SEQ ID NO: 96 - RNA, 22 nt, Homo sapiens
cagugcaaugaugaaagggcau

SEQ ID NO: 97 - RNA, 22 nt, Homo sapiens
accguggcuuucgauuguuacu

SEQ ID NO: 98 - RNA, 22 nt, Homo sapiens
uaacagucuacagccauggucg

SEQ ID NO: 99 - RNA, 22 nt, Homo sapiens
uuugguccccuucaaccagcug

SEQ ID NO: 100 - RNA, 23 nt, Homo sapiens
agcugguguugugaaucaggccg

SEQ ID NO: 101 - RNA, 22 nt, Homo sapiens
ucuacagugcacgugucuccag

SEQ ID NO: 102 - RNA, 21 nt, Homo sapiens
uaccacaggguagaaccacgg

SEQ ID NO: 103 - RNA, 22 nt, Homo sapiens
cagugguuuuacccuaugguag

SEQ ID NO: 104 - RNA, 22 nt, Homo sapiens
ggugcagugcugcaucucuggu

SEQ ID NO: 105 - RNA, 21 nt, Homo sapiens
ugagaugaagcacuguagcuc

SEQ ID NO: 106 - RNA, 23 nt, Homo sapiens
guccaguuuucccaggaaucccu

SEQ ID NO: 107 - RNA, 22 nt, Homo sapiens
ugagaacugaauuccauggguu

SEQ ID NO: 108 - RNA, 22 nt, Homo sapiens
ccucugaaauucaguucuucag

SEQ ID NO: 109 - RNA, 22 nt, Homo sapiens
ugagaacugaauuccauaggcu

SEQ ID NO: 110 - RNA, 22 nt, Homo sapiens
aaguucuguuauacacucaggc

SEQ ID NO: 111 - RNA, 22 nt, Homo sapiens
ucagugcaucacagaacuuugu

SEQ ID NO: 112 - RNA, 23 nt, Homo sapiens
ucuggcuccgugucuucacuccc

SEQ ID NO: 113 - RNA, 21 nt, Homo sapiens
agggagggacgggggcugugc

SEQ ID NO: 114 - RNA, 22 nt, Homo sapiens
ucucccaacccuuguaccagug

SEQ ID NO: 115 - RNA, 22 nt, Homo sapiens
cugguacaggccugggggacag

SEQ ID NO: 116 - RNA, 21 nt, Homo sapiens
cuagacugaagcuccuugagg

SEQ ID NO: 117 - RNA, 21 nt, Homo sapiens
ucagugcaugacagaacuugg

SEQ ID NO: 118 - RNA, 23 nt, Homo sapiens
uuaaugcuaaucgugauaggggu

SEQ ID NO: 119 - RNA, 22 nt, Homo sapiens
cuccuacauauuagcauuaaca

SEQ ID NO: 120 - RNA, 23 nt, Homo sapiens
aacauucaacgcugucggugagu

SEQ ID NO: 121 - RNA, 22 nt, Homo sapiens
aacauucaaccugucggugagu

SEQ ID NO: 122 - RNA, 22 nt, Homo sapiens
aaccaucgaccguugaguggac

SEQ ID NO: 123 - RNA, 22 nt, Homo sapiens
uggacggagaacugauaagggu

SEQ ID NO: 124 - RNA, 22 nt, Homo sapiens
uggagagaaaggcaguuccuga

SEQ ID NO: 125 - RNA, 22 nt, Homo sapiens
aggggcuggcuuuccucugguc

SEQ ID NO: 126 - RNA, 22 nt, Homo sapiens
caaagaauucuccuuuugggcu

SEQ ID NO: 127 - RNA, 22 nt, Homo sapiens
gcccaaaggugaauuuuuuggg

SEQ ID NO: 128 - RNA, 21 nt, Homo sapiens
caucccuugcaugguggaggg

SEQ ID NO: 129 - RNA, 22 nt, Homo sapiens
ugauauguuugauauauuaggu

SEQ ID NO: 130 - RNA, 23 nt, Homo sapiens
caacggaaucccaaaagcagcug

SEQ ID NO: 131 - RNA, 22 nt, Homo sapiens
gcugcgcuuggauuucgucccc

SEQ ID NO: 132 - RNA, 21 nt, Homo sapiens
cugaccuaugaauugacagcc

SEQ ID NO: 133 - RNA, 22 nt, Homo sapiens
cugccaauuccauaggucacag

SEQ ID NO: 134 - RNA, 22 nt, Homo sapiens
aacuggccuacaaagucccagu

SEQ ID NO: 135 - RNA, 22 nt, Homo sapiens
ugggucuuugcgggcgagauga

SEQ ID NO: 136 - RNA, 22 nt, Homo sapiens
cgggguuuugagggcgagauga

SEQ ID NO: 137 - RNA, 22 nt, Homo sapiens
aacuggcccucaaagucccgcu

SEQ ID NO: 138 - RNA, 22 nt, Homo sapiens
uguaacagcaacuccaugugga

SEQ ID NO: 139 - RNA, 21 nt, Homo sapiens
uagcagcacagaaauauuggc

SEQ ID NO: 140 - RNA, 22 nt, Homo sapiens
ccaauauuggcugugcugcucc

SEQ ID NO: 141 - RNA, 22 nt, Homo sapiens
uucaccaccuucuccacccagc

SEQ ID NO: 142 - RNA, 22 nt, Homo sapiens
acaguagucugcacauugguua

SEQ ID NO: 143 - RNA, 23 nt, Homo sapiens
cccaguguucagacuaccuguuc

SEQ ID NO: 144 - RNA, 23 nt, Homo sapiens
cccaguguuuagacuaucuguuc

SEQ ID NO: 145 - RNA, 22 nt, Homo sapiens
caucuuacugggcagcauugga

SEQ ID NO: 146 - RNA, 22 nt, Homo sapiens
uaauacugccugguaaugauga

SEQ ID NO: 147 - RNA, 22 nt, Homo sapiens
cgucuuacccagcaguguuugg

SEQ ID NO: 148 - RNA, 23 nt, Homo sapiens
uaauacugccggguaaugaugga

SEQ ID NO: 149 - RNA, 22 nt, Homo sapiens
uggaauguaaggaagugugugg

SEQ ID NO: 150 - RNA, 22 nt, Homo sapiens
cugugcgugugacagcggcuga

SEQ ID NO: 151 - RNA, 21 nt, Homo sapiens
uaacagucuccagucacggcc

SEQ ID NO: 152 - RNA, 22 nt, Homo sapiens
ugccugucuacacuugcugugc

SEQ ID NO: 153 - RNA, 22 nt, Homo sapiens
acagcaggcacagacaggcagu

SEQ ID NO: 154 - RNA, 21 nt, Homo sapiens
uugugcuugaucuaaccaugu

SEQ ID NO: 155 - RNA, 22 nt, Homo sapiens
accuggcauacaauguagauuu

SEQ ID NO: 156 - RNA, 23 nt, Homo sapiens
agcuacauugucugcuggguuuc

SEQ ID NO: 157 - RNA, 22 nt, Homo sapiens
cucaguagccaguguagauccu

SEQ ID NO: 158 - RNA, 21 nt, Homo sapiens
agcuacaucuggcuacugggu

SEQ ID NO: 159 - RNA, 22 nt, Homo sapiens
cguguauuugacaagcugaguu

SEQ ID NO: 160 - RNA, 22 nt, Homo sapiens
ugucaguuugucaaauacccca

SEQ ID NO: 161 - RNA, 21 nt, Homo sapiens
caagucacuagugguuccguu

SEQ ID NO: 162 - RNA, 23 nt, Homo sapiens
aaaauggugcccuagugacuaca

SEQ ID NO: 163 - RNA, 21 nt, Homo sapiens
agggcccccccucaauccugu

SEQ ID NO: 164 - RNA, 23 nt, Homo sapiens
cagugcaauaguauugucaaagc

SEQ ID NO: 165 - RNA, 23 nt, Homo sapiens
cagugcaaugauauugucaaagc

SEQ ID NO: 166 - RNA, 22 nt, Homo sapiens
aaaagcuggguugagagggcga

SEQ ID NO: 167 - RNA, 21 nt, Homo sapiens
cacauuacacggucgaccucu

SEQ ID NO: 168 - RNA, 20 nt, Homo sapiens
acugccccaggugcugcugg

SEQ ID NO: 169 - RNA, 23 nt, Homo sapiens
cgcauccccuagggcauuggugu

SEQ ID NO: 170 - RNA, 22 nt, Homo sapiens
cuggcccucucugcccuuccgu

SEQ ID NO: 171 - RNA, 21 nt, Homo sapiens
gccccugggccuauccuagaa

SEQ ID NO: 172 - RNA, 22 nt, Homo sapiens
cuagguauggucccagggaucc

SEQ ID NO: 173 - RNA, 23 nt, Homo sapiens
ugagcgccucgacgacagagccg

SEQ ID NO: 174 - RNA, 23 nt, Homo sapiens
ucccuguccuccaggagcucacg

SEQ ID NO: 175 - RNA, 22 nt, Homo sapiens
uuaucagaaucuccagggguac

SEQ ID NO: 176 - RNA, 22 nt, Mus musculus
uaaugccccuaaaaauccuuau

SEQ ID NO: 177 - RNA, 22 nt, Homo sapiens
agggacuuucaggggcagcugu

SEQ ID NO: 178 - RNA, 22 nt, Homo sapiens
acuguugcuaauaugcaacucu

SEQ ID NO: 179 - RNA, 22 nt, Homo sapiens
aauugcacuuuagcaaugguga

SEQ ID NO: 180 - RNA, 22 nt, Homo sapiens
auauaauacaaccugcuaagug

SEQ ID NO: 181 - RNA, 22 nt, Homo sapiens
cuuagcagguuguauuaucauu

SEQ ID NO: 182 - RNA, 22 nt, Homo sapiens
cuccugacuccagguccugugu

SEQ ID NO: 183 - RNA, 21 nt, Homo sapiens
acuggacuuggagucagaagg

SEQ ID NO: 184 - RNA, 22 nt, Homo sapiens
acuggacuuagggucagaaggc

SEQ ID NO: 185 - RNA, 23 nt, Homo sapiens
ugaggggcagagagcgagacuuu

SEQ ID NO: 186 - RNA, 23 nt, Homo sapiens
aaugacacgaucacucccguuga

SEQ ID NO: 187 - RNA, 22 nt, Homo sapiens
aucgggaaugucguguccgccc

SEQ ID NO: 188 - RNA, 22 nt, Homo sapiens
aggcaguguauuguuagcuggc

SEQ ID NO: 189 - RNA, 22 nt, Homo sapiens
cagccacaacuacccugccacu

SEQ ID NO: 190 - RNA, 22 nt, Homo sapiens
uuuugcgauguguuccuaauau

SEQ ID NO: 191 - RNA, 22 nt, Homo sapiens
aacuguuugcagaggaaacuga

SEQ ID NO: 192 - RNA, 22 nt, Homo sapiens
cucaucugcaaagaaguaagug

SEQ ID NO: 193 - RNA, 22 nt, Homo sapiens
aagacgggaggaaagaagggag

SEQ ID NO: 194 - RNA, 22 nt, Homo sapiens
ucaggcucaguccccucccgau

SEQ ID NO: 195 - RNA, 22 nt, Homo sapiens
gucauacacggcucuccucucu

SEQ ID NO: 196 - RNA, 22 nt, Homo sapiens
gugacaucacauauacggcagc

SEQ ID NO: 197 - RNA, 22 nt, Homo sapiens
ugaaacauacacgggaaaccuc

SEQ ID NO: 198 - RNA, 21 nt, Homo sapiens
cagcagcacacugugguuugu

SEQ ID NO: 199 - RNA, 22 nt, Homo sapiens
caaaccacacugugguguuaga

SEQ ID NO: 200 - RNA, 22 nt, Homo sapiens
ugauugguacgucuguggguag

SEQ ID NO: 201 - RNA, 18 nt, Homo sapiens
ugcuuccuuucagagggu

SEQ ID NO: 202 - RNA, 22 nt, Homo sapiens
ccucccacacccaaggcuugca

SEQ ID NO: 203 - RNA, 22 nt, Homo sapiens
caugccuugaguguaggaccgu

SEQ ID NO: 204 - RNA, 22 nt, Homo sapiens
ccucccacacccaaggcuugca

SEQ ID NO: 205 - RNA, 23 nt, Homo sapiens
agugccugagggaguaagagccc

SEQ ID NO: 206 - RNA, 22 nt, Homo sapiens
ugucuuacucccucaggcacau

SEQ ID NO: 207 - RNA, 19 nt, Homo sapiens
aggcacggugucagcaggc

SEQ ID NO: 208 - RNA, 20 nt, Homo sapiens
guccgcucggcgguggccca

SEQ ID NO: 209 - RNA, 22 nt, Homo sapiens
cacgcucaugcacacacccaca

SEQ ID NO: 210 - RNA, 21 nt, Homo sapiens
aagccugcccggcuccucggg

SEQ ID NO: 211 - RNA, 21 nt, Homo sapiens
ugagcuaaaugugugcuggga

SEQ ID NO: 212 - RNA, 21 nt, Homo sapiens
uggguuuacguugggagaacu

SEQ ID NO: 213 - RNA, 22 nt, Homo sapiens
guucucccaacguaagcccagc

SEQ ID NO: 214 - RNA, 23 nt, Homo sapiens
aucgcugcgguugcgagcgcugu

SEQ ID NO: 215 - RNA, 21 nt, Homo sapiens
aauggcgccacuaggguugug

SEQ ID NO: 216 - RNA, 24 nt, Homo sapiens
ugccugggucucuggccugcgcgu

SEQ ID NO: 217 - RNA, 21 nt, Homo sapiens
uccgguucucagggcuccacc

SEQ ID NO: 218 - RNA, 23 nt, Homo sapiens
aaggagcuuacaaucuagcuggg

SEQ ID NO: 219 - RNA, 22 nt, Homo sapiens
caacuagacugugagcuucuag

SEQ ID NO: 220 - RNA, 20 nt, Homo sapiens
cggcucugggucugugggga

SEQ ID NO: 221 - RNA, 23 nt, Homo sapiens
uccaguaccacgugucagggcca

SEQ ID NO: 222 - RNA, 22 nt, Homo sapiens
uauaccucaguuuuaucaggug

SEQ ID NO: 223 - RNA, 20 nt, Homo sapiens
guagaggagauggcgcaggg

SEQ ID NO: 224 - RNA, 21 nt, Homo sapiens
uccucuucucccuccucccag

SEQ ID NO: 225 - RNA, 24 nt, Homo sapiens
uggggagcugaggcucugggggug

SEQ ID NO: 226 - RNA, 23 nt, Homo sapiens
uggaagacuagugauuuuguugu

SEQ ID NO: 227 - RNA, 23 nt, Homo sapiens
uaaggugcaucuagugcaguuag

SEQ ID NO: 228 - RNA, 22 nt, Homo sapiens
ugcccuaaaugccccuucuggc

SEQ ID NO: 229 - RNA, 23 nt, Homo sapiens
uauggcuuuucauuccuauguga

SEQ ID NO: 230 - RNA, 22 nt, Homo sapiens
auguagggcuaaaagccauggg

SEQ ID NO: 231 - RNA, 23 nt, Homo sapiens
uguaguguuuccuacuuuaugga

SEQ ID NO: 232 - RNA, 21 nt, Homo sapiens
cauaaaguagaaagcacuacu

SEQ ID NO: 233 - RNA, 22 nt, Homo sapiens
gugugcggaaaugcuucugcua

SEQ ID NO: 234 - RNA, 22 nt, Homo sapiens
ucgugucuuguguugcagccgg

SEQ ID NO: 235 - RNA, 22 nt, Homo sapiens
ggcuacaacacaggacccgggc

SEQ ID NO: 236 - RNA, 22 nt, Homo sapiens
uagguaguuuccuguuguuggg

SEQ ID NO: 237 - RNA, 22 nt, Homo sapiens
ucgacagcacgacacugccuuc

SEQ ID NO: 238 - RNA, 22 nt, Homo sapiens
uucccuuugucauccuaugccu

SEQ ID NO: 239 - RNA, 22 nt, Homo sapiens
uaaucucagcuggcaacuguga

SEQ ID NO: 240 - RNA, 22 nt, Homo sapiens
gaggguuggguggaggcucucc

SEQ ID NO: 241 - RNA, 22 nt, Homo sapiens
uaacacugucugguaacgaugu

SEQ ID NO: 242 - RNA, 22 nt, Homo sapiens
caucuuaccggacagugcugga

SEQ ID NO: 243 - RNA, 23 nt, Homo sapiens
ucccccaggugugauucugauuu

SEQ ID NO: 244 - RNA, 22 nt, Homo sapiens
uuauaauacaaccugauaagug

SEQ ID NO: 245 - RNA, 22 nt, Homo sapiens
cuuaucagauuguauuguaauu

SEQ ID NO: 246 - RNA, 21 nt, Homo sapiens
uaugugccuuuggacuacauc

SEQ ID NO: 247 - RNA, 21 nt, Homo sapiens
gcaguccaugggcauauacac

SEQ ID NO: 248 - RNA, 22 nt, Homo sapiens
aagcccuuaccccaaaaagcau

SEQ ID NO: 249 - RNA, 22 nt, Homo sapiens
uuugguccccuucaaccagcua

SEQ ID NO: 250 - RNA, 21 nt, Homo sapiens
ugauauguuugauauuggguu

SEQ ID NO: 251 - RNA, 23 nt, Homo sapiens
ucucacacagaaaucgcacccgu

SEQ ID NO: 252 - RNA, 23 nt, Homo sapiens
uaaauuucaccuuucugagaagg

SEQ ID NO: 253 - RNA, 18 nt, Homo sapiens
uucacagggaggugucau

SEQ ID NO: 254 - RNA, 21 nt, Homo sapiens
ugaguuggccaucugagugag

SEQ ID NO: 255 - RNA, 22 nt, Homo sapiens
uugugucaauaugcgaugaugu

SEQ ID NO: 256 - RNA, 25 nt, Homo sapiens
agggaucgcgggcggguggcggccu

SEQ ID NO: 257 - RNA, 23 nt, Homo sapiens
ggcagguucucacccucucuagg

SEQ ID NO: 258 - RNA, 22 nt, Homo sapiens
cuugguucagggagggucccca

SEQ ID NO: 259 - RNA, 22 nt, Homo sapiens
acuccagccccacagccucagc

SEQ ID NO: 260 - RNA, 22 nt, Homo sapiens
uggagugugacaaugguguuug

SEQ ID NO: 261 - RNA, 22 nt, Rattus norvegicus
ucggauccgucugagcuuggcu

SEQ ID NO: 262 - RNA, 22 nt, Homo sapiens
ugugacugguugaccagagggg

SEQ ID NO: 263 - RNA, 23 nt, Homo sapiens
uuauugcuuaagaauacgcguag

SEQ ID NO: 264 - RNA, 22 nt, Homo sapiens
aaaguucugagacacuccgacu

SEQ ID NO: 265 - RNA, 22 nt, Homo sapiens
ugguuuaccgucccacauacau

SEQ ID NO: 266 - RNA, 22 nt, Homo sapiens
acuuuaacauggaagugcuuuc

SEQ ID NO: 267 - RNA, 21 nt, Homo sapiens
gaacggcuucauacaggaguu

SEQ ID NO: 268 - RNA, 22 nt, Homo sapiens
uuauaaagcaaugagacugauu

SEQ ID NO: 269 - RNA, 22 nt, Homo sapiens
uccgucucaguuacuuuauagc

SEQ ID NO: 270 - RNA, 22 nt, Homo sapiens
gcugacuccuaguccagggcuc

SEQ ID NO: 271 - RNA, 24 nt, Homo sapiens
aauccuuggaaccuaggugugagu

SEQ ID NO: 272 - RNA, 22 nt, Homo sapiens
agaucgaccguguuauauucgc

SEQ ID NO: 273 - RNA, 22 nt, Homo sapiens
gccugcugggguggaaccuggu

SEQ ID NO: 274 - RNA, 21 nt, Homo sapiens
aucauagaggaaaauccacgu

SEQ ID NO: 275 - RNA, 22 nt, Homo sapiens
guagauucuccuucuaugagua

SEQ ID NO: 276 - RNA, 21 nt, Homo sapiens
aucauagaggaacauccacuu

SEQ ID NO: 277 - RNA, 22 nt, Homo sapiens
guggauauuccuucuaugguua

SEQ ID NO: 278 - RNA, 21 nt, Homo sapiens
aacauagaggaaauuucacgu

SEQ ID NO: 279 - RNA, 21 nt, Homo sapiens
ugguagacuauggaacguagg

SEQ ID NO: 280 - RNA, 22 nt, Homo sapiens
uauguaacaugguccacuaacu

SEQ ID NO: 281 - RNA, 22 nt, Homo sapiens
gaauguugcucggugaaccccu

SEQ ID NO: 282 - RNA, 21 nt, Homo sapiens
uaguagaccguauagcguacg

SEQ ID NO: 283 - RNA, 22 nt, Homo sapiens
uauguaacacgguccacuaacc

SEQ ID NO: 284 - RNA, 22 nt, Homo sapiens
cagcagcaauucauguuuugaa

SEQ ID NO: 285 - RNA, 23 nt, Homo sapiens
ucuuggaguaggucauugggugg

SEQ ID NO: 286 - RNA, 21 nt, Homo sapiens
cuggauggcuccuccaugucu

SEQ ID NO: 287 - RNA, 22 nt, Homo sapiens
aucaugaugggcuccucggugu

SEQ ID NO: 288 - RNA, 22 nt, Homo sapiens
uuuugcaauauguuccugaaua

SEQ ID NO: 289 - RNA, 22 nt, Homo sapiens
uugggaucauuuugcauccaua

SEQ ID NO: 290 - RNA, 23 nt, Homo sapiens
uagugcaauauugcuuauagggu

SEQ ID NO: 291 - RNA, 22 nt, Homo sapiens
acccuaucaauauugucucugc

SEQ ID NO: 292 - RNA, 21 nt, Homo sapiens
gcaguccaugggcauauacac

SEQ ID NO: 293 - RNA, 22 nt, Homo sapiens
uccuguacugagcugccccgag

SEQ ID NO: 294 - RNA, 22 nt, Homo sapiens
aaucguacagggucauccacuu

SEQ ID NO: 295 - RNA, 22 nt, Homo sapiens
caaccuggaggacuccaugcug

SEQ ID NO: 296 - RNA, 22 nt, Homo sapiens
aguggggaacccuuccaugagg

SEQ ID NO: 297 - RNA, 22 nt, Homo sapiens
ugaaggucuacugugugccagg

SEQ ID NO: 298 - RNA, 22 nt, Homo sapiens
uuguacaugguaggcuuucauu

SEQ ID NO: 299 - RNA, 22 nt, Homo sapiens
aaacaaacauggugcacuucuu

SEQ ID NO: 300 - RNA, 23 nt, Homo sapiens
uaauccuugcuaccugggugaga

SEQ ID NO: 301 - RNA, 22 nt, Homo sapiens
augcaccugggcaaggauucug

SEQ ID NO: 302 - RNA, 22 nt, Homo sapiens
aauccuuugucccugggugaga

SEQ ID NO: 303 - RNA, 22 nt, Homo sapiens
aaugcaccugggcaaggauuca

SEQ ID NO: 304 - RNA, 23 nt, Homo sapiens
uagcagcgggaacaguucugcag

SEQ ID NO: 305 - RNA, 22 nt, Homo sapiens
ggagaaauuauccuuggugugu

SEQ ID NO: 306 - RNA, 23 nt, Homo sapiens
ucggggaucaucaugucacgaga

SEQ ID NO: 307 - RNA, 22 nt, Homo sapiens
aaacauucgcggugcacuucuu

SEQ ID NO: 308 - RNA, 21 nt, Homo sapiens
ugacaacuauggaugagcucu

SEQ ID NO: 309 - RNA, 22 nt, Homo sapiens
gagcuuauucauaaaagugcag

SEQ ID NO: 310 - RNA, 21 nt, Homo sapiens
agggggaaaguucuauagucc

SEQ ID NO: 311 - RNA, 22 nt, Homo sapiens
augcugacauauuuacuagagg

SEQ ID NO: 312 - RNA, 22 nt, Homo sapiens
auaauacaugguuaaccucuuu

SEQ ID NO: 313 - RNA, 22 nt, Homo sapiens
uacccauugcauaucggaguug

SEQ ID NO: 314 - RNA, 23 nt, Homo sapiens
ugucacucggcucggcccacuac

SEQ ID NO: 315 - RNA, 22 nt, Homo sapiens
uuugugaccugguccacuaacc

SEQ ID NO: 316 - RNA, 22 nt, Homo sapiens
ugagaccucuggguucugagcu

SEQ ID NO: 317 - RNA, 23 nt, Homo sapiens
cugggaucuccggggucuugguu

SEQ ID NO: 318 - RNA, 21 nt, Homo sapiens
cgcgggugcuuacugacccuu

SEQ ID NO: 319 - RNA, 23 nt, Homo sapiens
cgggucggaguuagcucaagcgg

SEQ ID NO: 320 - RNA, 22 nt, Rattus norvegicus
uacaguacugugauaacugaag

SEQ ID NO: 321 - RNA, 23 nt, Rattus norvegicus
uacccuguagauccgaauuugug

SEQ ID NO: 322 - RNA, 23 nt, Rattus norvegicus
uacccuguagaaccgaauuugug

SEQ ID NO: 323 - RNA, 22 nt, Rattus norvegicus
acaggugagguucuugggagcc

SEQ ID NO: 324 - RNA, 24 nt, Rattus norvegicus
ucccugagacccuuuaaccuguga

SEQ ID NO: 325 - RNA, 22 nt, Rattus norvegicus
acggguuaggcucuugggagcu

SEQ ID NO: 326 - RNA, 21 nt, Rattus norvegicus
ucacagucaaccggucucuuu

SEQ ID NO: 327 - RNA, 22 nt, Rattus norvegicus
aagcccuuaccccaaaaagcau

SEQ ID NO: 328 - RNA, 23 nt, Rattus norvegicus
agcugguguugugaaucaggccg

SEQ ID NO: 329 - RNA, 23 nt, Rattus norvegicus
uguaguguuuccuacuuuaugga

SEQ ID NO: 330 - RNA, 21 nt, Rattus norvegicus
cauaaaguagaaagcacuacu

SEQ ID NO: 331 - RNA, 23 nt, Rattus norvegicus
gcaaagcacagggccugcagaga

SEQ ID NO: 332 - RNA, 22 nt, Homo sapiens
acuccagccccacagccucagc

SEQ ID NO: 333-340 - skipped (8 entries)

SEQ ID NO: 341 - RNA, 21 nt, Rattus norvegicus
ugagaugaagcacuguagcuc

SEQ ID NO: 342 - RNA, 22 nt, Rattus norvegicus
ugagaacugaauuccauggguu

SEQ ID NO: 343 - RNA, 22 nt, Rattus norvegicus
gugugcggaaaugcuucugcua

SEQ ID NO: 344 - RNA, 22 nt, Rattus norvegicus
ucagugcacuacagaacuuugu

SEQ ID NO: 345 - RNA, 22 nt, Rattus norvegicus
ucagugcaucacagaacuuugu

SEQ ID NO: 346 - RNA, 21 nt, Rattus norvegicus
cuagacugaggcuccuugagg

SEQ ID NO: 347 - RNA, 25 nt, Rattus norvegicus
uuuggcaaugguagaacucacaccg

SEQ ID NO: 348 - RNA, 22 nt, Rattus norvegicus
ucgugucuuguguugcagccgg

SEQ ID NO: 349 - RNA, 21 nt, Rattus norvegicus
caucccuugcaugguggaggg

SEQ ID NO: 350 - RNA, 23 nt, Rattus norvegicus
uaaggugcaucuagugcagauag

SEQ ID NO: 351 - RNA, 22 nt, Rattus norvegicus
ugauauguuugauauauuaggu

SEQ ID NO: 352 - RNA, 22 nt, Rattus norvegicus
uagguaguuuccuguuguuggg

SEQ ID NO: 353 - RNA, 22 nt, Rattus norvegicus
uucaccaccuucuccacccagc

SEQ ID NO: 354 - RNA, 22 nt, Rattus norvegicus
acaguagucugcacauugguua

SEQ ID NO: 355 - RNA, 23 nt, Rattus norvegicus
uaauacugccggguaaugaugga

SEQ ID NO: 356 - RNA, 22 nt, Rattus norvegicus
uucccuuugucauccuaugccu

SEQ ID NO: 357 - RNA, 22 nt, Rattus norvegicus
cugugcgugugacagcggcuga

SEQ ID NO: 358 - RNA, 22 nt, Rattus norvegicus
uagcuuaucagacugauguuga

SEQ ID NO: 359 - RNA, 21 nt, Rattus norvegicus
agcuacaucuggcuacugggu

SEQ ID NO: 360 - RNA, 21 nt, Rattus norvegicus
aucacauugccagggauuucc

SEQ ID NO: 361 - RNA, 21 nt, Rattus norvegicus
aucacauugccagggauuacc

SEQ ID NO: 362 - RNA, 22 nt, Rattus norvegicus
uucaaguaauccaggauaggcu

SEQ ID NO: 363 - RNA, 23 nt, Rattus norvegicus
uagcaccauuugaaaucaguguu

SEQ ID NO: 364 - RNA, 22 nt, Rattus norvegicus
uagcaccauuugaaaucgguua

SEQ ID NO: 365 - RNA, 22 nt, Rattus norvegicus
aaaagcuggguugagagggcga

SEQ ID NO: 366 - RNA, 22 nt, Rattus norvegicus
cagcagcaauucauguuuugga

SEQ ID NO: 367 - RNA, 23 nt, Rattus norvegicus
cgcauccccuagggcauuggugu

SEQ ID NO: 368 - RNA, 22 nt, Rattus norvegicus
cuagguauggucccagggaucc

SEQ ID NO: 369 - RNA, 22 nt, Rattus norvegicus
uuuuucauuauugcuccugacc

SEQ ID NO: 370 - RNA, 23 nt, Rattus norvegicus
ucccuguccuccaggagcucacg

SEQ ID NO: 371 - RNA, 22 nt, Rattus norvegicus
gcugaccccuaguccagugcuu

SEQ ID NO: 372 - RNA, 22 nt, Rattus norvegicus
uucacaaagcccauacacuuuc

SEQ ID NO: 373 - RNA, 24 nt, Rattus norvegicus
ucccugaggagcccuuugagccug

SEQ ID NO: 374 - RNA, 22 nt, Rattus norvegicus
uuaucagaaucuccagggguac

SEQ ID NO: 375 - RNA, 22 nt, Rattus norvegicus
aacacaccuguucaaggauuca

SEQ ID NO: 376 - RNA, 23 nt, Rattus norvegicus
uguaaacaauuccuaggcaaugu

SEQ ID NO: 377 - RNA, 22 nt, Rattus norvegicus
uaauacugucugguaaugccgu

SEQ ID NO: 378 - RNA, 22 nt, Rattus norvegicus
uuuugcgauguguuccuaauau

SEQ ID NO: 379 - RNA, 22 nt, Rattus norvegicus
ugaaacauacacgggaaaccuc

SEQ ID NO: 380 - RNA, 22 nt, Rattus norvegicus
aaugcaccugggcaaggguuca

SEQ ID NO: 381 - RNA, 23 nt, Rattus norvegicus
uagcagcgggaacaguacugcag

SEQ ID NO: 382 - RNA, 22 nt, Rattus norvegicus
ugugacagauugauaacugaaa

SEQ ID NO: 383 - RNA, 22 nt, Rattus norvegicus
ccuguugaacaacugaacccaa

SEQ ID NO: 384 - RNA, 22 nt, Rattus norvegicus
uacaguuguucaaccaguuacu

SEQ ID NO: 385 - RNA, 22 nt, Rattus norvegicus
uacgucaucgucgucaucguua

SEQ ID NO: 386 - RNA, 21 nt, Rattus norvegicus
aauggcgccacuaggguugug

SEQ ID NO: 387 - RNA, 23 nt, Rattus norvegicus
ugacaccugccacccagcccaag

SEQ ID NO: 388 - RNA, 21 nt, Rattus norvegicus
ucaauggcugaggugaggcac

SEQ ID NO: 389 - RNA, 21 nt, Rattus norvegicus
uguucagacugguguccauca

SEQ ID NO: 390 - RNA, 22 nt, Rattus norvegicus
ugcggggcuagggcuaacagca

SEQ ID NO: 391 - RNA, 22 nt, Rattus norvegicus
uaacugcaacagcucucaguau

SEQ ID NO: 392 - RNA, 22 nt, Rattus norvegicus
uaacugcaacaucucucaguau

SEQ ID NO: 393 - RNA, 22 nt, Rattus norvegicus
ugagguaguaaguuguauuguu

SEQ ID NO: 394 - RNA, 21 nt, Rattus norvegicus
ugauauguuugauauuagguu

SEQ ID NO: 395 - RNA, 23 nt, Rattus norvegicus
gcuucuccuggcucuccucccuu

SEQ ID NO: 396 - RNA, 20 nt, Rattus norvegicus
guggugugcuaguuacuuuu

SEQ ID NO: 397 - RNA, 23 nt, Rattus norvegicus
ugagcgccucgacgacagagcca

SEQ ID NO: 398 - RNA, 22 nt, Rattus norvegicus
cccugaacuaggggucuggaga

SEQ ID NO: 399 - RNA, 25 nt, Rattus norvegicus
ucccugaggagcccuuugagccuga

SEQ ID NO: 400 - RNA, 21 nt, Rattus norvegicus
ugugaugugugcauguacaug

SEQ ID NO: 401 - RNA, 22 nt, Rattus norvegicus
gaaagacaccauacugaauaga

SEQ ID NO: 402 - DNA, 48 bp, Rattus norvegicus
gctgtactgacttgatgaaagtacttttgaacccttttccatctgatg

SEQ ID NO: 403 - RNA, 22 nt, Rattus norvegicus
cuauacaaucuauugccuuccc

SEQ ID NO: 404 - RNA, 22 nt, Rattus norvegicus
acuguacaggccacugccuugc

SEQ ID NO: 405 - RNA, 22 nt, Rattus norvegicus
cugcgcaagcuacugccuugcu

SEQ ID NO: 406 - RNA, 22 nt, Rattus norvegicus
ccgcacuguggguacuugcugc

SEQ ID NO: 407 - RNA, 22 nt, Rattus norvegicus
caaauucguaucuaggggaaua

SEQ ID NO: 408 - RNA, 22 nt, Rattus norvegicus
cagauucgauucuaggggaaua

SEQ ID NO: 409 - RNA, 22 nt, Rattus norvegicus
acucuuucccuguugcacuacu

SEQ ID NO: 410 - RNA, 22 nt, Rattus norvegicus
uauagggauuggagccguggcg

SEQ ID NO: 411 - RNA, 23 nt, Rattus norvegicus
ucuggcuccgugucuucacuccc

SEQ ID NO: 412 - RNA, 22 nt, Rattus norvegicus
cgaaucauuauuugcugcucua

SEQ ID NO: 413 - RNA, 22 nt, Rattus norvegicus
ccaguauugacugugcugcuga

SEQ ID NO: 414 - RNA, 22 nt, Rattus norvegicus
acugcagugagggcacuuguag

SEQ ID NO: 415 - RNA, 22 nt, Rattus norvegicus
acugcccuaagugcuccuucug

SEQ ID NO: 416 - RNA, 22 nt, Rattus norvegicus
gcugcacuuggauuucguuccc

SEQ ID NO: 417 - RNA, 23 nt, Rattus norvegicus
cccaguguuuagacuaccuguuc

SEQ ID NO: 418 - RNA, 22 nt, Rattus norvegicus
uggaauguaaggaagugugugg

SEQ ID NO: 419 - RNA, 22 nt, Rattus norvegicus
ugccugucuacacuugcugugc

SEQ ID NO: 420 - RNA, 22 nt, Rattus norvegicus
aaacaugguuccgucaagcacc

SEQ ID NO: 421 - RNA, 23 nt, Rattus norvegicus
gugccuacugagcugauaucagu

SEQ ID NO: 422 - RNA, 22 nt, Rattus norvegicus
ccuguucuccauuacuuggcuc

SEQ ID NO: 423 - RNA, 22 nt, Rattus norvegicus
caucaaaguggaggcccucucu

SEQ ID NO: 424 - RNA, 22 nt, Rattus norvegicus
uauacauacacacauacccaua

SEQ ID NO: 425 - RNA, 22 nt, Rattus norvegicus
acugauuucuuuugguguucag

SEQ ID NO: 426 - RNA, 22 nt, Rattus norvegicus
gcugguuucauauggugguuua

SEQ ID NO: 427 - RNA, 22 nt, Rattus norvegicus
ugaccgauuucuccugguguuc

SEQ ID NO: 428 - RNA, 22 nt, Rattus norvegicus
cuuucagucggauguuugcagc

SEQ ID NO: 429 - RNA, 22 nt, Rattus norvegicus
cugggauguggauguuuacguc

SEQ ID NO: 430 - RNA, 22 nt, Rattus norvegicus
cugggagaggguuguuuacucc

SEQ ID NO: 431 - RNA, 22 nt, Rattus norvegicus
cuuucagucggauguuuacagc

SEQ ID NO: 432 - RNA, 21 nt, Rattus norvegicus
aaacaugaagcgcugcaacac

SEQ ID NO: 433 - RNA, 21 nt, Rattus norvegicus
ccucugggcccuuccuccagu

SEQ ID NO: 434 - RNA, 22 nt, Rattus norvegicus
auauaauacaaccugcuaagug

SEQ ID NO: 435 - RNA, 22 nt, Rattus norvegicus
cuccugacuccagguccugugu

SEQ ID NO: 436 - RNA, 21 nt, Rattus norvegicus
acuggacuuggagucagaagg

SEQ ID NO: 437 - RNA, 21 nt, Rattus norvegicus
aucgggaaugucguguccgcc

SEQ ID NO: 438 - RNA, 21 nt, Rattus norvegicus
uauacauacacgcacacauag

SEQ ID NO: 439 - RNA, 22 nt, Rattus norvegicus
auauacauacacacaccuacac

SEQ ID NO: 440 - RNA, 22 nt, Rattus norvegicus
auauacauacacacaccaacac

SEQ ID NO: 441 - RNA, 22 nt, Rattus norvegicus
gaguauuguuuccacugccugg

SEQ ID NO: 442 - RNA, 22 nt, Rattus norvegicus
cucacagcucugguccuuggag

SEQ ID NO: 443 - RNA, 22 nt, Rattus norvegicus
cacagcucccaucucagaacaa

SEQ ID NO: 444 - RNA, 22 nt, Rattus norvegicus
gucucggugcaaggacuggagg

SEQ ID NO: 445 - RNA, 21 nt, Rattus norvegicus
aucucuuugagcgccucacuc

SEQ ID NO: 446 - RNA, 20 nt, Rattus norvegicus
aggcagugcgaccuggcucg

SEQ ID NO: 447 - RNA, 18 nt, Rattus norvegicus
aucucgcuggggccucca

SEQ ID NO: 448 - RNA, 21 nt, Rattus norvegicus
cagugcaauuaaaagggggaa

SEQ ID NO: 449 - RNA, 22 nt, Rattus norvegicus
cuguugccacuaaccucaaccu

SEQ ID NO: 450 - RNA, 20 nt, Rattus norvegicus
cggcucugggucugugggga

SEQ ID NO: 451 - RNA, 24 nt, Rattus norvegicus
gauugcugugcgugcggaaucgac

SEQ ID NO: 452 - RNA, 22 nt, Rattus norvegicus
uauaccucaguuuuaucaggug

SEQ ID NO: 453 - RNA, 20 nt, Rattus norvegicus
guagaggagauggcgcaggg

SEQ ID NO: 454 - RNA, 22 nt, Rattus norvegicus
auaaagcuagauaaccgaaagu

SEQ ID NO: 455 - RNA, 22 nt, Rattus norvegicus
caagcucgugucuguggguccg

SEQ ID NO: 456 - RNA, 22 nt, Rattus norvegicus
cacuagauugugagcuccugga

SEQ ID NO: 457 - RNA, 22 nt, Rattus norvegicus
ugauagacgccaauuuggguag

SEQ ID NO: 458 - RNA, 22 nt, Rattus norvegicus
caagcucguuucuaugggucug

SEQ ID NO: 459 - DNA, 57 bp, Rattus norvegicus
ctaaaatagctggaattaccggcagattggtagtggtgagcctatggttttctgaag

SEQ ID NO: 460 - RNA, 23 nt, Homo sapiens
uuaaugcuaaucgugauaggggu

SEQ ID NO: 461 - RNA, 22 nt, Homo sapiens
ugagguaguagguuguauaguu

SEQ ID NO: 462 - RNA, 22 nt, Homo sapiens
agagguaguagguugcauaguu

SEQ ID NO: 463 - RNA, 22 nt, Homo sapiens
ugagguaguagauuguauaguu

SEQ ID NO: 464 - RNA, 22 nt, Homo sapiens
ugagguaguaguuuguacaguu

SEQ ID NO: 465 - RNA, 22 nt, Homo sapiens
aacccguagauccgaacuugug

SEQ ID NO: 466 - RNA, 23 nt, Homo sapiens
agcagcauuguacagggcuauga

SEQ ID NO: 467 - RNA, 23 nt, Homo sapiens
aaaagugcuuacagugcagguag

SEQ ID NO: 468 - RNA, 21 nt, Homo sapiens
uaaagugcugacagugcagau

SEQ ID NO: 469 - RNA, 24 nt, Homo sapiens
ucccugagacccuuuaaccuguga

SEQ ID NO: 470 - RNA, 22 nt, Homo sapiens
ucccugagacccuaacuuguga

SEQ ID NO: 471 - RNA, 22 nt, Homo sapiens
ucguaccgugaguaauaaugcg

SEQ ID NO: 472 - RNA, 22 nt, Homo sapiens
ucggauccgucugagcuuggcu

SEQ ID NO: 473 - RNA, 22 nt, Homo sapiens
uaacagucuacagccauggucg

SEQ ID NO: 474 - RNA, 22 nt, Homo sapiens
ugugacugguugaccagagggg

SEQ ID NO: 475 - RNA, 23 nt, Homo sapiens
agcugguguugugaaucaggccg

SEQ ID NO: 476 - RNA, 22 nt, Homo sapiens
ucuacagugcacgugucuccag

SEQ ID NO: 477 - RNA, 21 nt, Homo sapiens
uaccacaggguagaaccacgg

SEQ ID NO: 478 - RNA, 22 nt, Homo sapiens
cagugguuuuacccuaugguag

SEQ ID NO: 479 - RNA, 21 nt, Homo sapiens
ugagaugaagcacuguagcuc

SEQ ID NO: 480 - RNA, 23 nt, Homo sapiens
guccaguuuucccaggaaucccu

SEQ ID NO: 481 - RNA, 22 nt, Homo sapiens
ugagaacugaauuccauaggcu

SEQ ID NO: 482 - RNA, 23 nt, Homo sapiens
ucuggcuccgugucuucacuccc

SEQ ID NO: 483 - RNA, 22 nt, Homo sapiens
uagcagcacaucaugguuuaca

SEQ ID NO: 484 - RNA, 23 nt, Homo sapiens
caaagugcuuacagugcagguag

SEQ ID NO: 485 - RNA, 23 nt, Homo sapiens
aacauucaacgcugucggugagu

SEQ ID NO: 486 - RNA, 22 nt, Homo sapiens
caaagaauucuccuuuugggcu

SEQ ID NO: 487 - RNA, 23 nt, Homo sapiens
caacggaaucccaaaagcagcug

SEQ ID NO: 488 - RNA, 22 nt, Homo sapiens
ugggucuuugcgggcgagauga

SEQ ID NO: 489 - RNA, 22 nt, Homo sapiens
aacuggcccucaaagucccgcu

SEQ ID NO: 490 - RNA, 21 nt, Homo sapiens
uagcagcacagaaauauuggc

SEQ ID NO: 491 - RNA, 22 nt, Homo sapiens
uucaccaccuucuccacccagc

SEQ ID NO: 492 - RNA, 22 nt, Homo sapiens
acaguagucugcacauugguua

SEQ ID NO: 493 - RNA, 23 nt, Homo sapiens
ugugcaaaucuaugcaaaacuga

SEQ ID NO: 494 - RNA, 23 nt, Homo sapiens
ugugcaaauccaugcaaaacuga

SEQ ID NO: 495 - RNA, 22 nt, Homo sapiens
uucccuuugucauccuaugccu

SEQ ID NO: 496 - RNA, 23 nt, Homo sapiens
uaaagugcuuauagugcagguag

SEQ ID NO: 497 - RNA, 22 nt, Homo sapiens
cugugcgugugacagcggcuga

SEQ ID NO: 498 - RNA, 22 nt, Homo sapiens
uagcuuaucagacugauguuga

SEQ ID NO: 499 - RNA, 22 nt, Homo sapiens
acagcaggcacagacaggcagu

SEQ ID NO: 500 - RNA, 23 nt, Homo sapiens
agcuacauugucugcuggguuuc

SEQ ID NO: 501 - RNA, 21 nt, Homo sapiens
agcuacaucuggcuacugggu

SEQ ID NO: 502 - RNA, 22 nt, Homo sapiens
uggcucaguucagcaggaacag

SEQ ID NO: 503 - RNA, 22 nt, Homo sapiens
uucaaguaauccaggauaggcu

SEQ ID NO: 504 - RNA, 21 nt, Homo sapiens
uucaaguaauucaggauaggu

SEQ ID NO: 505 - RNA, 21 nt, Homo sapiens
uucacaguggcuaaguuccgc

SEQ ID NO: 506 - RNA, 21 nt, Homo sapiens
uucacaguggcuaaguucugc

SEQ ID NO: 507 - RNA, 22 nt, Homo sapiens
cacuagauugugagcuccugga

SEQ ID NO: 508 - RNA, 22 nt, Homo sapiens
aaggagcucacagucuauugag

SEQ ID NO: 509 - RNA, 22 nt, Homo sapiens
uagcaccaucugaaaucgguua

SEQ ID NO: 510 - RNA, 22 nt, Homo sapiens
uagcaccauuugaaaucgguua

SEQ ID NO: 511 - RNA, 22 nt, Homo sapiens
uguaaacauccuacacucagcu

SEQ ID NO: 512 - RNA, 23 nt, Homo sapiens
uguaaacauccuacacucucagc

SEQ ID NO: 513 - RNA, 21 nt, Homo sapiens
aggcaagaugcuggcauagcu

SEQ ID NO: 514 - RNA, 22 nt, Homo sapiens
aaaagcuggguugagagggcga

SEQ ID NO: 515 - RNA, 21 nt, Homo sapiens
cacauuacacggucgaccucu

SEQ ID NO: 516 - RNA, 20 nt, Homo sapiens
acugccccaggugcugcugg

SEQ ID NO: 517 - RNA, 22 nt, Homo sapiens
cuggcccucucugcccuuccgu

SEQ ID NO: 518 - RNA, 21 nt, Homo sapiens
gccccugggccuauccuagaa

SEQ ID NO: 519 - RNA, 23 nt, Homo sapiens
ugagcgccucgacgacagagccg

SEQ ID NO: 520 - RNA, 23 nt, Homo sapiens
ucucacacagaaaucgcacccgu

SEQ ID NO: 521 - RNA, 22 nt, Homo sapiens
gcugacuccuaguccagggcuc

SEQ ID NO: 522 - RNA, 22 nt, Homo sapiens
uggcagugucuuagcugguugu

SEQ ID NO: 523 - RNA, 24 nt, Homo sapiens
aauccuuggaaccuaggugugagu

SEQ ID NO: 524 - RNA, 22 nt, Homo sapiens
uaaugccccuaaaaauccuuau

SEQ ID NO: 525 - RNA, 22 nt, Homo sapiens
uuauaauacaaccugauaagug

SEQ ID NO: 526 - RNA, 22 nt, Homo sapiens
auauaauacaaccugcuaagug

SEQ ID NO: 527 - RNA, 21 nt, Homo sapiens
aucauagaggaaaauccacgu

SEQ ID NO: 528 - RNA, 21 nt, Homo sapiens
aacauagaggaaauuccacgu

SEQ ID NO: 529 - RNA, 21 nt, Homo sapiens
uaguagaccguauagcguacg

SEQ ID NO: 530 - RNA, 23 nt, Homo sapiens
aaugacacgaucacucccguuga

SEQ ID NO: 531 - RNA, 21 nt, Homo sapiens
ugucuugcaggccgucaugca

SEQ ID NO: 532 - RNA, 22 nt, Homo sapiens
aacuguuugcagaggaaacuga

SEQ ID NO: 533 - RNA, 23 nt, Homo sapiens
uagugcaauauugcuuauagggu

SEQ ID NO: 534 - RNA, 22 nt, Homo sapiens
aagacgggaggaaagaagggag

SEQ ID NO: 535 - RNA, 22 nt, Homo sapiens
ucaggcucaguccccucccgau

SEQ ID NO: 536 - RNA, 22 nt, Homo sapiens
gucauacacggcucuccucucu

SEQ ID NO: 537 - RNA, 22 nt, Homo sapiens
uccuguacugagcugccccgag

SEQ ID NO: 538 - RNA, 22 nt, Homo sapiens
aguggggaacccuuccaugagg

SEQ ID NO: 539 - RNA, 22 nt, Homo sapiens
aaacaaacauggugcacuucuu

SEQ ID NO: 540 - RNA, 22 nt, Homo sapiens
ccucccacacccaaggcuugca

SEQ ID NO: 541 - RNA, 22 nt, Homo sapiens
caugccuugaguguaggaccgu

SEQ ID NO: 542 - RNA, 22 nt, Homo sapiens
ggagaaauuauccuuggugugu

SEQ ID NO: 543 - RNA, 22 nt, Homo sapiens
cacgcucaugcacacacccaca

SEQ ID NO: 544 - RNA, 22 nt, Homo sapiens
gagcuuauucauaaaagugcag

SEQ ID NO: 545 - RNA, 22 nt, Homo sapiens
augcugacauauuuacuagagg

SEQ ID NO: 546 - RNA, 21 nt, Homo sapiens
uccgguucucagggcuccacc

SEQ ID NO: 547 - RNA, 23 nt, Homo sapiens
aaggagcuuacaaucuagcuggg

SEQ ID NO: 548 - RNA, 22 nt, Homo sapiens
ugcggggcuagggcuaacagca

SEQ ID NO: 549 - RNA, 21 nt, Homo sapiens
cgcgggugcuuacugacccuu

SEQ ID NO: 550 - RNA, 23 nt, Homo sapiens
cgggucggaguuagcucaagcgg

SEQ ID NO: 551 - RNA, 22 nt, Homo sapiens
uauugcacuugucccggccugu

SEQ ID NO: 552 - RNA, 23 nt, Homo sapiens
caaagugcuguucgugcagguag

SEQ ID NO: 553 - RNA, 22 nt, Homo sapiens
aacccguagauccgaucuugug

SEQ ID NO: 554 - DNA, 57 bp, Homo sapiens
gatgaccccaggtaactctgagtgtgtcgctgatgccatcaccgcagcgctctgacc

SEQ ID NO: 555 - RNA, 22 nt, Homo sapiens
ugagguaguagguugugugguu

SEQ ID NO: 556 - RNA, 23 nt, Homo sapiens
uacccuguagaaccgaauuugug

SEQ ID NO: 557 - RNA, 22 nt, Homo sapiens
uuuccggcucgcgugggugugu

SEQ ID NO: 558 - RNA, 24 nt, Homo sapiens
agccugauuaaacacaugcucuga

SEQ ID NO: 559 - RNA, 20 nt, Homo sapiens
ucacuguucagacaggcgga

SEQ ID NO: 560 - RNA, 20 nt, Homo sapiens
ugagcccuguccucccgcag

SEQ ID NO: 561 - RNA, 22 nt, Homo sapiens
acccgucccguucguccccgga

SEQ ID NO: 562 - RNA, 22 nt, Homo sapiens
acggguuaggcucuugggagcu

SEQ ID NO: 563 - RNA, 18 nt, Homo sapiens
aucccaccucugccacca

SEQ ID NO: 564 - RNA, 22 nt, Homo sapiens
cuuggcaccuagcaagcacuca

SEQ ID NO: 565 - RNA, 18 nt, Homo sapiens
gucccuguucaggcgcca

SEQ ID NO: 566 - RNA, 17 nt, Homo sapiens
ucccuguucgggcgcca

SEQ ID NO: 567 - RNA, 17 nt, Homo sapiens
gugggggagaggcuguc

SEQ ID NO: 568 - RNA, 19 nt, Homo sapiens
uggauuuuuggaucaggga

SEQ ID NO: 569 - RNA, 22 nt, Homo sapiens
cagugcaaugaugaaagggcau

SEQ ID NO: 570 - RNA, 23 nt, Homo sapiens
guccaguuuucccaggaaucccu

SEQ ID NO: 571 - RNA, 23 nt, Homo sapiens
ucuggcuccgugucuucacuccc

SEQ ID NO: 572 - RNA, 21 nt, Homo sapiens
cuagacugaagcuccuugagg

SEQ ID NO: 573 - RNA, 21 nt, Homo sapiens
ucgaggagcucacagucuagu

SEQ ID NO: 574 - RNA, 22 nt, Homo sapiens
accacugaccguugacuguacc

SEQ ID NO: 575 - RNA, 22 nt, Homo sapiens
aacuggcccucaaagucccgcu

SEQ ID NO: 576 - RNA, 22 nt, Homo sapiens
accaucgaccguugauuguacc

SEQ ID NO: 577 - RNA, 22 nt, Homo sapiens
acagcaggcacagacaggcagu

SEQ ID NO: 578 - RNA, 22 nt, Homo sapiens
aagcugccaguugaagaacugu

SEQ ID NO: 579 - RNA, 22 nt, Homo sapiens
ugccuacugagcugaaacacag

SEQ ID NO: 580 - RNA, 22 nt, Homo sapiens
ccuauucuugguuacuugcacg

SEQ ID NO: 581 - RNA, 21 nt, Homo sapiens
uucacaguggcuaaguuccgc

SEQ ID NO: 582 - RNA, 21 nt, Homo sapiens
uucacaguggcuaaguucugc

SEQ ID NO: 583 - RNA, 22 nt, Homo sapiens
cuuucagucggauguuugcagc

SEQ ID NO: 584 - RNA, 22 nt, Homo sapiens
uguaaacauccucgacuggaag

SEQ ID NO: 585 - RNA, 22 nt, Homo sapiens
cuuucagucggauguuuacagc

SEQ ID NO: 586 - RNA, 21 nt, Homo sapiens
aggcaagaugcuggcauagcu

SEQ ID NO: 587 - RNA, 23 nt, Homo sapiens
ucaagagcaauaacgaaaaaugu

SEQ ID NO: 588 - RNA, 22 nt, Homo sapiens
caaucacuaacuccacugccau

SEQ ID NO: 589 - RNA, 21 nt, Homo sapiens
acuggacuuggagucagaagg

SEQ ID NO: 590 - RNA, 22 nt, Homo sapiens
gaauguugcucggugaaccccu

SEQ ID NO: 591 - RNA, 22 nt, Homo sapiens
cagcagcaauucauguuuugaa

SEQ ID NO: 592 - RNA, 23 nt, Homo sapiens
aaugacacgaucacucccguuga

SEQ ID NO: 593 - RNA, 23 nt, Homo sapiens
ucuuggaguaggucauugggugg

SEQ ID NO: 594 - RNA, 22 nt, Homo sapiens
aaagugcuucucuuuggugggu

SEQ ID NO: 595 - RNA, 22 nt, Homo sapiens
aaacauucgcggugcacuucuu

SEQ ID NO: 596 - RNA, 22 nt, Homo sapiens
ucagcaaacauuuauugugugc

SEQ ID NO: 597 - RNA, 21 nt, Homo sapiens
aagccugcccggcuccucggg

SEQ ID NO: 598 - RNA, 21 nt, Homo sapiens
agggggaaaguucuauagucc

SEQ ID NO: 599 - RNA, 24 nt, Homo sapiens
aaagacauaggauagagucaccuc

SEQ ID NO: 600 - RNA, 23 nt, Homo sapiens
uauucauuuauccccagccuaca

SEQ ID NO: 601 - RNA, 17 nt, Homo sapiens
ucucgcuggggccucca

SEQ ID NO: 602 - RNA, 23 nt, Homo sapiens
ucugcucauaccccaugguuucu

SEQ ID NO: 603 - RNA, 22 nt, Homo sapiens
ugagaccucuggguucugagcu

SEQ ID NO: 604 - RNA, 22 nt, Homo sapiens
uauaccucaguuuuaucaggug

SEQ ID NO: 605 - RNA, 23 nt, Homo sapiens
caaagugcuguucgugcagguag

SEQ ID NO: 606 - RNA, 22 nt, Homo sapiens
caacaaaucacagucugccaua

SEQ ID NO: 607 - DNA, 60 bp, Homo sapiens
cctggatgatgatagcaaatgctgactgaacatgaaggtcttaattagctctaactgact

SEQ ID NO: 608 - DNA, 57 bp, Homo sapiens; identical to SEQ ID NO: 554

SEQ ID NO: 609 - RNA, 22 nt, Homo sapiens
ugcggggcuagggcuaacagca

SEQ ID NO: 610 - RNA, 22 nt, Homo sapiens
cuguugccacuaaccucaaccu

(19) United States
(12) Patent Application Publication
(10) Pub. No.: US 2014/0234263 A1
(43) Pub. Date: Aug. 21, 2014

(54) CELLULAR AND MOLECULAR THERAPIES

(71) Applicant: The University Court of the University of Glasgow, Glasgow (GB)

(72) Inventor: Paul Shiels, Glasgow (GB)

(73) Assignee: The University Court of the University of Glasgow, Glasgow (GB)

(21) Appl. No.: 13/766,637

(22) Filed: Feb. 13, 2013

Related U.S. Application Data
(63) Continuation of application No. PCT/IB2011/002048, filed on Aug. 12, 2011.
(60) Provisional application No. 61/373,715, filed on Aug. 13, 2010.
(60) Provisional application No. 61/380,766, filed on Sep. 8, 2010.

(51) Int. Cl. A61K 35/39; A61K 35/28

(52) U.S. Cl. CPC A61K 35/39; A61K 35/28; A61K 2035/124
USPC 424/93.7

(57) ABSTRACT

The present invention provides, among other things, improved compositions and methods for the treatment of tissue damage (e.g., acute or chronic) and related diseases, disorders or conditions based on the use of pathfinder cells, extracellular secretomes thereof, and/or pathfinder cell-associated microRNAs. In some embodiments, the present invention provides a method for treating tissue damage (e.g., acute or chronic) comprising a step of administering a population of cells to an individual suffering from a disease, disorder or condition characterized by acute damage to one or more tissues, wherein the cells are originated from an adult tissue and wherein the cells induce tissue repair, regeneration, remodeling, reconstitution or differentiation. In some embodiments, the present invention provides a method for treating inflammation comprising a step of administering a population of cells, or extracellular secretomes thereof, to an individual suffering from a disease, disorder or condition characterized by inflammation of one or more tissues, wherein the cells are originated from an adult tissue and wherein the cells induce an anti-inflammatory response.

### CROSS REFERENCE TO RELATED APPLICATIONS

This application is a continuation of international application No. PCT/IB2011/002048 filed on Aug. 12, 2011, which claims the benefit of U.S. Provisional Patent Application Ser. Nos. 61/373,715, filed Aug. 13, 2010 and 61/380,766, filed Sep. 8, 2010, the entirety of each of which is incorporated herein by reference.

This application relates to US application entitled “Therapeutic Uses of Microvesicles and Related MicroRNAs” filed on even date, the entirety of which is incorporated herein by reference.

### SEQUENCE LISTING

The present specification makes reference to a Sequence Listing (submitted electronically as a .txt file named “Sequence Listing.txt on Feb. 13, 2013). The .txt file was generated on Feb. 13, 2013 and is 89.2 kb in size. The entire contents of the Sequence Listing are herein incorporated by reference.

### BACKGROUND

Acute tissue damage may cause serious diseases and dangerous health conditions. For example, much acute tissue damage is caused by ischemic conditions. Organ ischemia occurs when blood flow to an organ is interrupted, usually by a blood clot or a severe drop in blood pressure. In ischemic strokes, a lack of oxygen flow to the brain can result in apoptosis and necrosis of brain tissue leading to an infarction. Similar to cardiovascular ischemia, brain ischemia can be caused by various factors such as blood clots, thrombosis, embolism, blockage by atherosclerotic plaques, or other obstructions in the vasculature. Hypercholesterolemia, hypertension, diabetes, and obesity, among other things, have been identified as risk factors for ischemic strokes. Ischemic strokes are a leading cause of death of human beings worldwide. In the United States, strokes strikes 700,000 people annually. Of these stroke patients, 40% will die, the equivalent of one death every three minutes. Acute tissue damage may also occur when a tissue is injured or under stressful or traumatic condition, resulting in acute organ failure.

Traditionally, acute tissue damage is treated by so-called secondary treatment such as organ transplantation and medical and surgical therapies. Although these therapies alleviate symptoms, and may even improve survival, none can reverse the disease process and directly repair the lasting damage. Thus, the cure of diseases, disorders or conditions associated with acute tissue damage remain a major unmet medical need.

### SUMMARY OF THE INVENTION

The present invention provides, among other things, cellular and molecular therapies for acute tissue damage and related diseases, disorders and conditions. The present invention encompasses the discovery that multipotent cells may be isolated from various adult tissues and these multipotent cells, also known as “pathfinder cells,” showed significant therapeutic effects in various acute tissue damage model in various tissues, including pancreas, kidney, heart and blood system. The present invention further encompasses the discovery that pathfinder cells contain certain specific microRNAs that may function as intercellular regulators involved in cell or tissue repair, regeneration, remodeling, reconstruction, reprogramming, or transdifferentiation, and the discovery that pathfinder cells or their extracellular secretomes (e.g., microvesicles) induce immune tolerance and thus are particularly useful in treating inflammation and suppressing, inhibiting or reducing transplantation associated stress. As shown in the Examples section below, the pathfinder cells induce immune tolerance at least by suppressing pro-inflammatory and/or anti-angiogenic cytokines or chemokines, such as, IL-4, IL-5, IL-6, IL-10, IL-12, IL-13, IL-17, GMCSF, TGF-β, TNF-α, IFN-γ, MCAF, and MIP1. It was found that pathfinder cells have unexpectedly distinct cytokine or chemokine profiles as compared to Mesenchymal stem cells (MSCs). For example, pathfinder cells according to the present invention reduce the expression or activity of IL-4, IL-6 and/or IL-10, while MSCs increase the expression or activity of IL-4, IL-6 and IL-10. Other distinctions are shown in Table 7. This discovery further confirms that pathfinder cells are distinct from mesenchymal stem cells.

Thus, in various embodiments, the present invention provides methods and compositions for treating or cure acute tissue damage and related diseases, disorders and conditions based on pathfinder cells and/or associated microRNAs.

In some embodiments, the present invention provides a method for treating acute tissue damage comprising a step of administering a population of cells to an individual suffering from a disease, disorder or condition characterized by acute damage to one or more tissues, wherein the cells are originated from an adult tissue and wherein the cells induce tissue repair, regeneration, remodeling, reconstitution or differentiation.

In some embodiments, the acute damage is to one or more tissues selected from the group consisting of kidney, heart, liver, lungs, pancreas, brain, intestine, bones, tendons, cornea, skin, muscle, veins, spinal cord, spleen, blood, and combinations thereof. In some embodiments, the acute damage is to kidney and/or heart.

In some embodiments, the acute damage is ischemic damage. In some embodiments, the acute damage is associated with tissue transplantation. In some embodiments, the acute damage is associated with exposure to radiation and/or chemicals (e.g., in the context of therapy or injury, such as chemical injury to the cornea, skin, or other tissues). In some embodiments, the acute damage is associated with an inflammatory condition.

In some embodiments, an inventive method according to the present invention is used to treat a disease, disorder or condition selected from the group consisting of myocardial infarct, acute renal failure, type I diabetes, and combination thereof. In some embodiments, the disease, disorder, or condition is characterized by cell apoptosis, aponecrosis, or necrosis. In some embodiments, the disease, disorder, or condition is characterized by inflammation of one or tissues. In some embodiments, the disease, disorder, or condition is associated with biological aging.

In some embodiments, the cells used in a method of the invention are originated from an adult tissue that is distinct from the damaged tissue. In some embodiments, the cells used in a method of the invention are originated from an adult tissue that is from a different species. In some embodiments, the cells used in a method of the invention are originated from a human adult tissue. In some embodiments, the cells used in a method of the invention are originated from a non-human adult tissue. In some embodiments, the cells used in a method of the invention are originated from an autologous adult tissue. In some embodiments, the cells used in a method of the invention are originated from a non-autologous adult tissue. In some embodiments, the adult tissue is selected from the group consisting of pancreas, kidney, breast, lymph node, liver, spleen, myometrium, peripheral blood, chord blood, and bone marrow, and combinations thereof.

In some embodiments, the cells used in a method according to the invention are first cultivated in a cell culture medium (e.g., serum-containing or serum-free) under conditions and time sufficient for cell proliferation. In some embodiments, the cell culture medium is a Matrigel free culture medium comprising serum. In some embodiments, the cells are first treated to reduce a telomeric attrition rate before the cultivating step.

In some embodiments, the population of cells is substantially homogenous. In some embodiments, at least 30% (e.g., at least 35%, 40%, 45%, 50%, 55%, 60%, 65%, 70%, 75%, 80%, 85%, 90%, 95%, or more) of the population of cells express one or more (e.g., two or more, three or more, four or more, five or more, six or more, seven or more, eight or more, or nine or more) markers selected from the group consisting of CD24, c-myc, HLA class 1 ABC, ICAM3, Nestin, Nanog, Oct4, Integrin a2+b1, Ngn3, and CD130. In some embodiments, the one or more markers comprise Oct4, Nanog, and c-myc. In some embodiments, at least 30% (e.g., at least 35%, 40%, 45%, 50%, 55%, 60%, 65%, 70%, 75%, 80%, 85%, 90%, 95%, or more) of the population of cells express Nestin. In some embodiments, at least 30% (e.g., at least 35%, 40%, 45%, 50%, 55%, 60%, 65%, 70%, 75%, 80%, 85%, 90%, 95%, or more) of the population of cells do not express at least one (e.g., at least two, at least three, at least four, at least five, at least six, at least seven, at least eight) marker(s) selected from the group consisting of CD34, CD105, VCAM1, CXCR2, CD44, CD73, ICAM1, and NCAM. In some embodiments, at least 30% (e.g., at least 35%, 40%, 45%, 50%, 55%, 60%, 65%, 70%, 75%, 80%, 85%, 90%, 95%, or more) of the population of cells do not express any of the markers selected from the group consisting of CD34, CD105, VCAM1, CXCR2, CD44, CD73, ICAM1, and NCAM. In some embodiments, the cells used in a method according to the present invention express one or more microRNAs as shown in Table 1. In some embodiments, the cells used in a method according to the present invention express one or more microRNAs as shown in Tables 6-10. In certain embodiments, the cells used in a method according to the present invention express one or more microRNAs as shown in Table 10.

In some embodiments, the population of cells used in a method constitutes a therapeutically effective amount of cells. In some embodiments, the therapeutically effective amount ranges from approximately 1×10⁶ to 3×10⁸ cells per kg body weight per dose.

In some embodiments, the cells are administered intravenously, intra-arterially, intramuscularly, subcutaneously, cutaenously, intradermally, intracranially, intrathecally, intrapleurally, intra-orbitally, intranasally, orally, intra alimentrally, colorectally, and/or intra-cerebrospinally.

In some embodiments, the cells are administered daily, three times a week, twice a week, weekly, biweekly, monthly, once every two months, once every three months, once every four months, once every five months, once every six months, or once every year.

In some embodiments, the cells are not administered in conjunction with an immunosuppressant.

In some embodiments, the present invention provides a method for treating tissue damage (e.g., acute tissue damage) comprising a step of administering to an individual suffering from a disease, disorder or condition characterized by tissue damage a therapeutically effective amount of one or more microRNAs having a sequence at least 70% (e.g., 75%, 80%, 85%, 90%, 95%, 96%, 97%, 98%, 99%) identical to any of SEQ ID NOs: 1 to 610 (or any one of SEQ ID NOs: 1 to 225). In some embodiments, the one or more microRNAs comprise a sequence identical to any of SEQ ID NOs: 1 to 610 (or any one of SEQ ID NOs: 1 to 225).

Among other things, the present invention may be used to facilitate organ transplantation (e.g., heart, kidney, liver, lung, pancreas, intestine, thymus, and skin transplantation). In some embodiments, the present invention provides a method of organ transplantation comprising a step of administering to an organ transplant recipient a population of cells, wherein the cells are originated from an adult tissue and wherein the cells induce tissue repair, regeneration, remodeling, reconstruction, reprogramming, or transdifferentiation. In some embodiments, the organ transplant recipient is a heart, kidney, liver, lung, pancreas, intestine, thymus, or skin transplant recipient.

The present invention also provides various pharmaceutical compositions based on cells and/or microRNAs described herein. In some embodiments, the present invention provides a pharmaceutical composition for tissue repair or regeneration comprising a substantially homogeneous population of cells and a pharmaceutically acceptable carrier, wherein the cells are originated from an adult tissue and wherein the cells induce tissue repair, regeneration, remodeling, reconstruction, reprogramming, or transdifferentiation. Various cells described above in connection with various methods may be used in various embodiments of pharmaceutical compositions.

In some embodiments, the present invention provides a pharmaceutical composition for tissue repair or regeneration comprising a therapeutically effective amount of one or more microRNAs having a sequence at least 70% (e.g., 75%, 80%, 85%, 90%, 95%, 96%, 97%, 98%, 99%) identical to any of SEQ ID NOs: 1 to 610 (or any one of SEQ ID NOs: 1 to 225) and a pharmaceutically acceptable carrier. In some embodiments, the one or more microRNAs have a sequence identical to any of SEQ ID NOs: 1 to 610 (or any one of SEQ ID NOs: 1 to 225).

The present invention is not limited to the treatment of acute tissue damage. It is contemplated that methods and compositions described herein may be used to treat tissue damage in general including both acute and chronic damage and related diseases, disorders, or conditions.

In some embodiments, the present invention provides a method for treating inflammation comprising a step of administering a population of cells, or extracellular secretomes thereof, to an individual suffering from a disease, disorder or condition characterized by inflammation of one or more tissues, wherein the cells are originated from an adult tissue and wherein the cells induce an anti-inflammatory response. In some embodiments, the extracellular secretoms comprise microvesicles.

In some embodiments, the cells induce increased IL-2 response. In some embodiments, the cells induce expansion of regulatory T cells, suppression of T cell responses and/or immune tolerance. In some embodiments, the expansion of regulatory T cells comprises increased level and/or activity of T regulatory cells. In certain embodiments, the cells induce decreased level and/or activity of cytotoxic T cells and/or helper T cells.

In some embodiments, the cells suppress pro-inflammatory and/or anti-angiogenic cytokine or chemokine response. In certain embodiments, the pro-inflammatory and/or anti-angiogenic cytokine or chemokine is selected from the group consisting of IL-4, IL-5, IL-6, IL-10, IL-12, IL-13, IL-17, GMCSF, TGF-β, TNF-α, IFN-γ, MCAF, MIP1, and combinations thereof. In some embodiments, the cells do not increase the expression or activity of IL-4, IL-6 and/or IL-10.

In some embodiments, the cells increase anti-inflammatory and/or pro-angiogenic cytokine or chemokine response. In certain embodiments, the anti-inflammatory and/or pro-angiogenic cytokine or chemokine is selected from IL-β, GSCF, IL-8 and combinations thereof.

In some embodiments, an inventive method according to the present invention is used to treat a disease, disorder or condition selected from acute inflammatory conditions; adult respiratory distress syndrome (ARDS); Airway hyperresponsiveness (AHR); allergy disorders; alopecia greata; ankylosing spondylitis; asthma; atopic inflammatory disorders; autoimmune thyroiditis; bronchial hyperreactivity; cancers; cardiovascular disorders; Chronic Obstructive Pulmonary disease (COPD); cirrhosis; Crohn's disease, Congestive Heart Failure (CHF); colitis; dermatitis; diabetes mellitus (type I); disorders associated with wound healing; end-stage renal disease (ESRD); eosinophilic esophagitis; HIV; Graves' disease; infectious diseases; inflammatory bowel disease (IBD); inflammatory complications of diabetes mellitus; inflammatory conditions of the skin, cardiovascular system, nervous system, liver, kidney and pancreas; juvenile rheumatoid arthritis; lupus-associated arthritis; metabolic syndrome; multiple sclerosis; muscle fatigue; myasthenia gravis; neurodegenerative diseases; osteoarthritis; primary billiary cirrhosis; psoriatic arthritis; respiratory disorders; rheumatoid arthritis; sarcoidosis; scleroderma; Sjogren's syndrome; Systemic Inflammatory Response Syndrome (SIRS)/sepsis; systemic lupus erythematosis; transplant rejection; ulcerative colitis; and/or vasculitis. In some embodiments, the inflammation is associated with Graft-versus-host disease (GVHD). In some embodiments, the inflammation is associated with autoimmune disease. In some embodiments, the inflammation is associated with chronic renal disease. In some embodiments, the inflammation is associated with osteoarthritis.

In some embodiments, the individual suffering from a disease, disorder or condition characterized by inflammation of one or more tissues is a mammal. In certain embodiments, the mammal is a human.

In some embodiments, the adult tissue from which the cells are originated is from a human. In some embodiments, the adult tissue is from a non-human mammal, e.g., selected from a rodent, a mouse, a rat, a rabbit, a monkey, a dog, a cat, a sheep, cattle, a primate, and/or a pig.

In some embodiments, the adult tissue is selected from the group consisting of bone marrow, liver, blood, heart, brain, gum, eye, skin, breast, chord blood, kidney, lymph node, myometrium, pancreas, peripheral blood, spleen, and combinations thereof.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### BRIEF DESCRIPTION OF THE DRAWINGS

The drawings are for illustration purposes only not for limitation.

FIG. 1 shows a graphical representation of serum creatinine levels in mice at day 14 post ischemia. Control mice (group A) were injected with saline, while experimental mice (groups B-D) received PDPCs according to various treatment regimens.

FIG. 2 depicts photomicrographs of kidney sections from control ischemic mice and PDPC-treated ischemic mice. Sections were stained for p16, p21, and SAβGAL (markers of cell stress and cell senescence) and TUNEL (a marker of apoptosis).

FIG. 3 shows graphs displaying relative levels of cytoplasmic p21 expression in heart tissues of control and PDPC-treated cardiac ischemic mice. From left to right, the bars in each group are (1) ischemic mice that were untreated; (2) ischemic mice that were injected with an NaCl solution; (3) ischemic mice that were treated with PDPCs; and (4) untreated, non-ischemic mice.

FIGS. 4 and 5 show exemplary graphical representations of serum creatinine levels in mice at day 14 post ischemia. Control mice (group A) were injected with saline, while experimental mice (groups B-D) received PDPCs according to various treatment regimens.

FIGS. 6 and 7 show exemplary graphical representations of serum creatinine levels adjusted for body weight in mice at day 14 post ischemia. Control mice (group A) were injected with saline, while experimental mice (groups B-D) received PDPCs according to various treatment regimens.

FIG. 8 shows exemplary graphs displaying measurement of various indicators of cardiac function in cardiac ischemic mice (controls and PDPC-treated). Fractional shortening (FS), left ventricle diastolic diameter (LVEDD), and left ventricle systolic diameter (LVESD) were measured before ischemia and at 1 and 2 weeks post-ischemia.

FIG. 9 shows an exemplary graph displaying measurement of fractional shortening (FS) in cardiac ischemic mice (controls and PDPC-treated).

FIG. 10 shows an exemplary graph displaying measurement of left ventricle diastolic diameter (LVEDD) in cardiac ischemic mice (controls and PDPC-treated).

FIG. 11 shows an exemplary graph displaying measurement of left ventricle systolic diameter (LVESD) in cardiac ischemic mice (controls and PDPC-treated).

FIGS. 12, 13, and 14 show exemplary graphs displaying blood glucose levels of NOD mice in groups 1, 2, and 3 respectively at various time points. Group 1 mice were untreated; group 2 mice were treated with PDPCs; and group 3 mice were treated with PDPCs and CsA (cyclosporine A, an immunosuppressant).

FIGS. 15A and 15B depict exemplary scanning electron microscopy pictures of sub-confluent rat PDPCs adapted for growth in medium with fetal bovine serum (FBS) depleted for bovine microvesicles. Nascent microvesicles can be seen at the surfaces of cells in both figures.

FIGS. 16 to 21 depict exemplary ELISA measurements of IL-1B, IL-2, IL-4, IL-5, IL-6, IL-7, IL-8, IL-10, IL-12, IL-13, IL-17, GCSF, GMCSF, IFN-g, MCAF, MIP-1B, and TNFa expression in response to incubation of MLR with PCs as compared to incubation of MLR with control cells.

FIGS. 22 to 34 depict exemplary ELISA measurements of IL-2, IL-4, IL-5, IL-6, IL-7, IL-8, IL-10, IL-13, and IL-17, GCSF, GMCSF, and MCAF release by each of Fractions 1-4 in a mixed lymphocyte reaction (MLR).

### DEFINITIONS

In order for the present invention to be more readily understood, certain terms are first defined below. Additional definitions for the following terms and other terms are set forth throughout the specification.

Acute: As used herein, the term “acute” when used in connection with tissue damage and related diseases, disorders, or conditions, has the meaning understood by any one skilled in the medical art. For example, the term typically refers to a disease, disorder, or condition in which there is sudden or severe onset of symptoms. In some embodiments, acute damage is due to an ischemic or traumatic event. Typically, the term “acute” is used in contrast to the term “chronic.”

Adult: As used herein, the term “adult” when used to describe tissues is not meant herein to imply that the tissues must be obtained from an adult individual, but rather, that the tissues are themselves fully developed or differentiated, rather than being in embryonic or undifferentiated form.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Autoimmune disorder: As used herein, the term “autoimmune disorder” refers to a disorder resulting from attack of a body's own tissue by its immune system. In some embodiments, autoimmune diseases is diabetes mellitus, multiple sclerosis, premature ovarian failure, scleroderma, Sjogren's disease, lupus, alopecia (baldness), polyglandular failure, Grave's disease, hypothyroidism, polymyosititis, Celiac disease, Crohn's disease, inflammatory bowel disease, ulcerative colitis, autoimmune hepatitis, hypopituitarism, Guillain-Barre syndrome, myocardititis, Addison's disease, autoimmune skin diseases (e.g., psoriasis), uveititis, pernicious anemia, polymyalgia rheumatica, Goodpasture's syndrome, hypoparathyroidism, Hashimoto's thyoriditis, Raynaud's phenomenon, polymyaglia rheumatica, and rheumatoid arthritis.

Autologous and non-autologus: As used herein, the term “autologous” means from the same organism. In the context of the present application, the term is used to mean that the population of cells and/or microvesicles referred to as “autologous” to each other do not contain any material which could be regarded as allogenic or xenogenic, that is to say derived from a “foreign” cellular source. As used herein, the term “non-autologous” means not from the same organism.

Cell culture: As used herein, the term “cell culture” or its equivalents shall mean one or more cells cultivated in a controlled environment. A cell culture typically pertains to an isolated collection of cells in a defined medium under controlled conditions. However, in certain circumstances, a cell culture may contain a single cell. Typically, a single cell culture is obtained from a larger culture by dilution. In other embodiments, a cell culture contains two or more cells. For such a cell culture, the culture may consist of cells of a significantly pure population. For example, in some embodiments, cells in a culture are clonal in nature. Clonal cells are derived from a single parental cell and typically contain identical genetic make-up. In some embodiments, however, at least one of the cells in a culture contains at least one genetic mutation. Alternatively, a culture may be characterized as a mixed cell culture, which contains multiple cell types. In some embodiments, a mixed cell culture is prepared by design. In other embodiments, a mixed cell culture is a result of a contamination. In the context of cell cultures, the terms “grown” “cultivated” “maintained” and the like, are used interchangeably herein, unless specifically stated otherwise.

Chronic: As used herein, the term “chronic,” when used in connection with tissue damage or related diseases, disorders, or conditions has the meaning as understood by any one skilled in the medical art. Typically, the term “chronic” refers to diseases, disorders, or conditions that involve persisting and/or recurring symptoms. Chronic diseases, disorders, or conditions typically develop over a long period of time. The term “chronic” is used in contrast to the term “acute.” In some embodiments, a chronic disease, disorder, or condition results from cell degeneration. In some embodiments, a chronic disease, disorder, or condition results from age-related cell degeneration.

Diabetes mellitus: As used herein, the term “diabetes mellitus” refers to a metabolic disease characterized by abnormally high levels of glucose in the blood, caused by an inherited inability to produce insulin (Type 1) or an acquired resistance to insulin (Type 2). Type 1 diabetes is a severe, chronic form of diabetes caused by insufficient production of insulin and resulting in abnormal metabolism of carbohydrates, fats, and proteins. The disease, which typically appears in childhood or adolescence, is characterized by increased sugar levels in the blood and urine, excessive thirst, frequent urination, acidosis, and wasting. Type 1 diabetes is also called insulin-dependent diabetes. Type 2 diabetes is a mild form of diabetes that typically appears first in adulthood and is exacerbated by obesity and an inactive lifestyle. This disease often has no symptoms, is usually diagnosed by tests that indicate glucose intolerance, and is treated with changes in diet and an exercise regimen. Type 2 diabetes is also called non-insulin-dependent diabetes.

Control: As used herein, the term “control” has its art-understood meaning of being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. In one experiment, the “test” (i.e., the variable being tested) is applied. In the second experiment, the “control,” the variable being tested is not applied. In some embodiments, a control is a historical control (i.e., of a test or assay performed previously, or an amount or result that is previously known). In some embodiments, a control is or comprises a printed or otherwise saved record. A control may be a positive control or a negative control. In some embodiments, a control is also referred to as a reference.

Cosmetic surgical procedure: As used herein, the term “cosmetic surgical procedure” refers to a procedure that is not directed to the therapy of a disease but is, rather, directed to the improvement of an individual's aesthetic appearance, particularly the appearance of the skin or hair of an individual. Examples of cosmetic surgical procedures include procedures that result in reduction in skin wrinkles, an increase in skin firmness, an increase in hair growth or shine, a reduction in grey hairs, a regrowth of hair in cases of baldness (especially male pattern baldness), reduction in hair growth (especially facial hair growth), an aesthetic enhancement of breast size or shape, and a reduction in cellulite.

Crude: As used herein, the term “crude,” when used in connection with a biological sample, refers to a sample which is in a substantially unrefined state. For example, a crude sample can be cell lysates or biopsy tissue sample. A crude sample may exist in solution or as a dry preparation.

Derivative thereof. As used herein, the term “derivative thereof,” when used in connection with microvesicles or cells, refers to a fraction or extract (especially those containing RNA and/or DNA and/or protein) of the original microvesicle or population of cells which retains at least some biological activity (especially the ability to induce differentiation and/or the ability to provide therapeutic benefit) of the original. The term also include complexed, encapsulated or formulated microvesicles or cells (for example, microvesicles that have been encapsulated, complexed or formulated to facilitate administration). Examples of derivatives include lysates, lyophilates and homogenates.

Dysfunction: As used herein, the term “dysfunction” refers to an abnormal function. Dysfunction of a molecule (e.g., a protein) can be caused by an increase or decrease of an activity associated with such molecule. Dysfunction of a molecule can be caused by defects associated with the molecule itself or other molecules that directly or indirectly interact with or regulate the molecule.

Functional: As used herein, a “functional” biological molecule is a biological molecule in a form in which it exhibits a property and/or activity by which it is characterized.

Functional derivative: As used herein, the term “functional derivative” denotes, in the context of a functional derivative of a nucleotide sequence (e.g., microRNA), a molecule that retains a biological activity (either function or structural) that is substantially similar to that of the original sequence. A functional derivative or equivalent may be a natural derivative or is prepared synthetically. Exemplary functional derivatives include nucleotide sequences having substitutions, deletions, or additions of one or more nucleotides, provided that the biological activity of the nucleic acids (e.g., microRNAs) is conserved.

Inflammation: As used herein, the term “inflammation” includes inflammatory conditions occurring in many disorders which include, but are not limited to: Systemic Inflammatory Response (SIRS); Alzheimer's Disease (and associated conditions and symptoms including: chronic neuroinflammation, glial activation; increased microglia; neuritic plaque formation; and response to therapy); Amyotropic Lateral Sclerosis (ALS), arthritis (and associated conditions and symptoms including, but not limited to: acute joint inflammation, antigen-induced arthritis, arthritis associated with chronic lymphocytic thyroiditis, collagen-induced arthritis, juvenile arthritis; rheumatoid arthritis, osteoarthritis, prognosis and streptococcus-induced arthritis, spondyloarthopathies, gouty arthritis), asthma (and associated conditions and symptoms, including: bronchial asthma; chronic obstructive airway disease; chronic obstructive pulmonary disease, juvenile asthma and occupational asthma); cardiovascular diseases (and associated conditions and symptoms, including atherosclerosis; autoimmune myocarditis, chronic cardiac hypoxia, congestive heart failure, coronary artery disease, cardiomyopathy and cardiac cell dysfunction, including: aortic smooth muscle cell activation; cardiac cell apoptosis; and immunomodulation of cardiac cell function; diabetes and associated conditions and symptoms, including autoimmune diabetes, insulin-dependent (Type 1) diabetes, diabetic periodontitis, diabetic retinopathy, and diabetic nephropathy); gastrointestinal inflammations (and related conditions and symptoms, including celiac disease, associated osteopenia, chronic colitis, Crohn's disease, inflammatory bowel disease and ulcerative colitis); gastric ulcers; hepatic inflammations such as viral and other types of hepatitis, cholesterol gallstones and hepatic fibrosis, HIV infection (and associated conditions and symptoms, including degenerative responses, neurodegenerative responses, and HIV associated Hodgkin's Disease), Kawasaki's Syndrome (and associated diseases and conditions, including mucocutaneous lymph node syndrome, cervical lymphadenopathy, coronary artery lesions, edema, fever, increased leukocytes, mild anemia, skin peeling, rash, conjunctiva redness, thrombocytosis; multiple sclerosis, nephropathies (and associated diseases and conditions, including diabetic nephropathy, endstage renal disease, acute and chronic glomerulonephritis, acute and chronic interstitial nephritis, lupus nephritis, Goodpasture's syndrome, hemodialysis survival and renal ischemic reperfusion injury), neurodegenerative diseases (and associated diseases and conditions, including acute neurodegeneration, induction of IL-1 in aging and neurodegenerative disease, IL-1 induced plasticity of hypothalamic neurons and chronic stress hyperresponsiveness), ophtlialmopathies (and associated diseases and conditions, including diabetic retinopathy, Graves' opthalmopathy, and uveitis, osteoporosis (and associated diseases and conditions, including alveolar, femoral, radial, vertebral or wrist bone loss or fracture incidence, postmenopausal bone loss, mass, fracture incidence or rate of bone loss), otitis media (adult or pediatric), pancreatitis or pancreatic acinitis, periodontal disease (and associated diseases and conditions, including adult, early onset and diabetic); pulmonary diseases, including chronic lung disease, chronic sinusitis, hyaline membrane disease, hypoxia and pulmonary disease in SIDS; restenosis of coronary or other vascular grafts; rheumatism including rheumatoid arthritis, rheumatic Aschoff bodies, rheumatic diseases and rheumatic myocarditis; thyroiditis including chronic lymphocytic thyroiditis; urinary tract infections including chronic prostatitis, chronic pelvic pain syndrome and urolithiasis. Immunological disorders, including autoimmune diseases, such as alopecia aerata, autoimmune myocarditis, Graves' disease, Graves opthalmopathy, lichen sclerosis, multiple sclerosis, psoriasis, systemic lupus erythematosus, systemic sclerosis, thyroid diseases (e.g. goiter and struma lymphomatosa (Hashimoto's thyroiditis, lymphadenoid goiter), sleep disorders and chronic fatigue syndrome and obesity (non-diabetic or associated with diabetes). Resistance to infectious diseases, such as Leishmaniasis, Leprosy, Lyme Disease, Lyme Carditis, malaria, cerebral malaria, meningitis, tubulointerstitial nephritis associated with malaria), which are caused by bacteria, viruses (e.g. cytomegalovirus, encephalitis, Epstein-Barr Virus, Human Immunodeficiency Virus, Influenza Virus) or protozoans (e.g., Plasmodium falciparum, trypanosomes). Response to trauma, including cerebral trauma (including strokes and ischemias, encephalitis, encephalopathies, epilepsy, perinatal brain injury, prolonged febrile seizures, SIDS and subarachnoid hemorrhage), low birth weight (e.g. cerebral palsy), lung injury (acute hemorrhagic lung injury, Goodpasture's syndrome, acute ischemic reperfusion), myocardial dysfunction, caused by occupational and environmental pollutants (e.g. susceptibility to toxic oil syndrome silicosis), radiation trauma, and efficiency of wound healing responses (e.g. burn or thermal wounds, chronic wounds, surgical wounds and spinal cord injuries). Hormonal regulation including fertility/fecundity, likelihood of a pregnancy, incidence of preterm labor, prenatal and neonatal complications including preterm low birth weight, cerebral palsy, septicemia, hypothyroidism, oxygen dependence, cranial abnormality, early onset menopause. A subject's response to transplant (rejection or acceptance), acute phase response (e.g. febrile response), general inflammatory response, acute respiratory distress response, acute systemic inflammatory response, wound healing, adhesion, immunoinflammatory response, neuroendocrine response, fever development and resistance, acute-phase response, stress response, disease susceptibility, repetitive motion stress, tennis elbow, and pain management and response.

Inducer: As used herein, the term “inducer” refers to any molecule or other substance capable of inducing a change in the fate of differentiation of a cell to which it is applied.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism such as a non-human animal.

Ischemia: As used herein, the term “ischemia” (also spelled “ischaemia”) typically refers to a restriction in blood or oxygen supply that may result in damage or dysfunction of a tissue. Ischemia may be caused by any of a variety of factors, such as factors in blood vessels, a blood clot, a severe drop in blood pressure, an increase in compartmental pressure, and/or trauma. The term “ischemia” as used herein also refers to local anemia in a given part of a body or tissue that may result, for example, from vasoconstriction, thrombosis, or embolism. Any tissue that normally receives a blood supply can experience ischemia.

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, about 95%, about 98%, about 99%, substantially 100%, or 100% of the other components with which they were initially associated. In some embodiments, isolated agents are more than about 80%, about 85%, about 90%, about 91%, about 92%, about 93%, about 94%, about 95%, about 96%, about 97%, about 98%, about 99%, substantially 100%, or 100% pure. As used herein, a substance is “pure” if it is substantially free of other components. As used herein, the term “isolated cell” refers to a cell not contained in a multi-cellular organism.

microRNA: As used herein, the term “microRNAs (miRNAs)” refers to post-transcriptional regulators that typically bind to complementary sequences in the three prime untranslated regions (3′ UTRs) of target messenger RNA transcripts (mRNAs), usually resulting in gene silencing. Typically, miRNAs are short ribonucleic acid (RNA) molecules, for example, 21 or 22 nucleotides long. The terms “microRNA” and “miRNA” are used interchangeably.

Microvesicle: As used herein, the term “microvesicle” refers to a membranaceus particle comprising fragments of plasma membrane derived from various cell types. Typically, microvesicles have a diameter (or largest dimension where the particle is not spheroid) of between about 10 nm to about 5000 nm (e.g., between about 50 nm and 1500 nm, between about 75 nm and 1500 nm, between about 75 nm and 1250 nm, between about 50 nm and 1250 nm, between about 30 nm and 1000 nm, between about 50 nm and 1000 nm, between about 100 nm and 1000 nm, between about 50 nm and 750 nm, etc.). Typically, at least part of the membrane of the microvesicle is directly obtained from a cell (also known as a donor cell). Microvesicles suitable for use in the present invention may originate from cells by membrane inversion, exocytosis, shedding, blebbing, and/or budding. Depending on the manner of generation (e.g., membrane inversion, exocytosis, shedding, or budding), the microvesicles contemplated herein may exhibit different surface/lipid characteristics. Alternative names for microvesicles include, but are not limited to, exosomes, ectosomses, membrane particles, exosome-like particles, and apoptotic vesicles. As used herein, an abbreviated form “MV” is sometime used to refer to microvesicle.

Pathfinder cells: As used herein, the term “pathfinder cells” refers to cells that have the capacity to induce or stimulate tissue repair, regeneration, remodeling, reconstitution, or differentiation. Typically, pathfinder cells induce or stimulate tissue or repair, regeneration, remodeling, reconstitution or differentiation without being a source of new tissue themselves. In some embodiments, pathfinder cells are also referred to as “progenitor cells.” As used herein, an abbreviated form “PC” is sometime used to refer to a pathfinder cell. Typically, pathfinder cells are originated from adult tissues, including primary cells directly isolated from adult tissues or cells grown from primary cells in a cell culture system. The term “adult” when used to describe tissues is not meant herein to imply that the tissues must be obtained from an adult individual, but rather, that the tissues are themselves fully developed, rather than being in embryonic form or derived from a fetus. Thus, it is specifically contemplated that pathfinder cells may be obtained from any developed individual (whether a child, adolescent, or adult), but are generally not obtained from embryos or fetuses. Pathfinder cells typically have one or more characteristics as described herein, such as expression of microRNAs, cell surface markers, and production of microvesicles.

Subject: As used herein, the term “subject” refers to a human or any non-human animal (e.g., mouse, rat, rabbit, dog, cat, cattle, swine, sheep, horse or primate). A human includes pre and post natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of the disease, disorder, and/or condition.

Susceptible to: An individual who is “susceptible to” a disease, disorder, and/or condition has not been diagnosed with the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Therapeutic agent: As used herein, the phrase “therapeutic agent” refers to any agent that, when administered to a subject, has a therapeutic effect and/or elicits a desired biological and/or pharmacological effect. In some embodiments, a therapeutic agent of the invention refers to a peptide inhibitor or derivatives thereof according to the invention.

Tissue: As used herein, the term “tissue” refers to an aggregation of morphologically similar cells and associated intercellular matter acting together to perform one or more specific functions in the body. In some embodiments, tissues fall into one of four basic types: muscle, nerve, epidermal, and connective. In some embodiments, a tissue is substantially solid, e.g., cells within the tissue are strongly associated with one another to form a solid. In some embodiments, a tissue is substantially non-solid, e.g, cells within the tissue are loosely associated with one another, or not at all physically associated with one another, but may be found in the same space, bodily fluid, etc. For example, blood cells are considered a tissue in non-solid form.

Transdifferentiation: As used herein, the term “transdifferentiation” refers to a process in which a non-stem cell transforms into a different type of cell, or an already differentiated stem cell creates cells outside its already established differentiation path. Typically, transdifferentiation include de- and then re-differentiation of adult cell types (or differentiated cell types).

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### DETAILED DESCRIPTION OF CERTAIN EMBODIMENTS

The present invention provides, among other things, improved compositions and methods for the treatment of tissue damage (e.g., acute or chronic) and related diseases, disorders or conditions based on the use of pathfinder cells, extracellular secretomes thereof, and/or pathfinder cell-associated microRNAs. In some embodiments, the present invention provides a method for treating tissue damage (e.g., acute or chronic) comprising a step of administering a population of cells to an individual suffering from a disease, disorder or condition characterized by acute damage to one or more tissues, wherein the cells are originated from an adult tissue and wherein the cells induce tissue repair, regeneration, remodeling, reconstitution or differentiation.

Various aspects of the invention are described in detail in the following sections. The use of sections is not meant to limit the invention. Each section can apply to any aspect of the invention. In this application, the use of “or” means “and/or” unless stated otherwise.

### I. Cells

Cells suitable for the present invention are typically multipotent or pluripotent and can induce tissue repair, regeneration, remodeling, reconstruction, reprogramming, or transdifferentiation in vivo and/or in vitro. Typically, the present invention utilizes cells originated from an adult tissue. Such cells are also referred to as “pathfinder cells” or “progenitor cells.” In this application, the terms “cells,” “pathfinder cells” and “progenitor cells” are used interchangeably.

As discussed in the Examples section, there is evidence that pathfinder cells may induce or stimulate host tissue repair, regeneration, remodeling, reconstitution, differentiation or transdifferentitaion without being a source of new tissue themselves. Without wishing to be bound by any theory, it is contemplated that pathfinder cells may induce changes within target tissue or cells to convert them into active repair mode by providing microRNAs and/or other components (e.g., membrane associated polypeptide, transcription factors, etc.) that will regulate expression of genes relating to, e.g., increased cell mobility, tissue remodeling and reprogramming, growth, angiogenesis, cell adhesion and cell signaling, etc.

Thus, according to the present invention, pathfinder cells or microRNAs from different tissues, cell types or organisms may be used. For example, cells suitable for the present invention may be originated from an adult tissue that may or may not be the same as the target tissue. In some embodiments, cells suitable for the present invention may be originated from autologous or non-autologous adult tissues. In some embodiments, cells suitable for the present invention may be originated from adult tissues obtained from different species. For example, cells suitable for the present invention may be originated from human or non-human adult tissues.

In some embodiments, cells suitable for the present invention may have any desirable origin, including endothelial, mesothelial, and ectothelial origins. Thus, suitable cells include those found in a gland, an organ, muscle, a structural tissue, etc. Suitable cells may be heterologous (or non-autologous) or autologous relative to recipient. For example, suitable cells may be derived from a tissue the same as or different than the recipient tissue (e.g., a diseased or damaged tissue to be treated). As a non-limiting example, kidney-derived cells may be used to stimulate repair of damaged cardiac tissue. In some embodiments, cells may be derived from a different organism (i.e., non-autologous). For example, a cell may be a porcine pancreatic cell, while the recipient is a human with damaged pancreatic or other tissue.

Cells suitable for the present invention may be isolated from any of a variety of tissue types, including, but not limited to, pancreas, kidney, lymph node, liver, spleen, myometrium, blood cells (including cells from peripheral blood and chord blood), and/or bone marrow.

Suitable cells may also be in any stage of their individual cellular age, ranging from just separated from their progenitor cell to a senescent or even dead cell. Thus, cells may include pre-apoptotic cells, or a cell committed to apoptosis.

Furthermore, it is contemplated that suitable cells also include non-diseased and diseased cells, wherein diseased cells may be affected by one or more pathogens and/or conditions. For example, a diseased cell may be infected with a virus, an intracellular parasite, or bacterium. In other examples, a diseased cell may be a metabolically diseased cell (e.g., due to genetic defect, due to an enzyme, receptor, and/or transporter dysfunction, or due to metabolic insult), a neoplastic cell, or cell that has one or more mutations that render the cell susceptible to uncontrolled cell growth. Similarly, cells may be native (e.g., obtained by biopsy), cultured (e.g., native, or immortalized), or treated. For example, cells may be chemically and/or mechanically treated, resulting in a cell that exhibits a cell-specific stress response. In some embodiments, suitable cells may be treated with a natural or synthetic ligand to which the cell has a receptor or otherwise complementary structure. In some embodiments, a cell may also be treated with a drug or compound that alters at least one of a metabolism, cell growth, cell division, cell structure, and/or secretion.

In some embodiments, suitable cells are recombinant cells. For example, recombinant cells may contain one or more nucleic acid molecules introduced by recombinant DNA technology. All known manners of introducing nucleic acids are deemed suitable for use herein (e.g., viral transfection, chemical transfection, electroporation, ballistic transfection, etc.). Where the nucleic is a DNA, it is contemplated that the DNA may be integrated into the genome of the cell, or that the DNA may reside as extrachromosomal unit within the cell. Such DNA may be employed as a template for RNA production, which may have regulatory and/or protein encoding function. Similarly where the nucleic acid is an RNA, such RNA may be used as a regulatory entity (e.g., via antisense or interference) and/or as a protein encoding entity. As used herein, nucleic acids encompass all known nucleic acid analogs (e.g., phosphorothioate analogs, peptide nucleic acid analogs, etc.)

In some embodiments, cells are obtained from freshly isolated or stored materials (e.g., biological fluids, tissues, organs, etc.). When cells are obtained from stored materials, such storage may include storage at a reduced temperature (e.g., 4° C.) or even storage in frozen form. Similarly, cells may also be obtained from an in vitro source, and most typically from cell or tissue culture (see the Cell Culture Condition section below), or even organ culture.

Cells may be isolated, for example, according to methods previously developed by the present inventor and available in the art, or variations thereof. See, e.g., Shiels et al. (2009) Stem Cells Dev., 18(10):1389-98 and International Patent Publications WO2006/120476 and WO2009/136168. For example, a tissue or a part of a tissue (e.g., pancreatic ducts) can be isolated from an adult animal and minced. The minced tissue can be seeded in a suitable culture medium and incubated. In some embodiments, pathfinder cells will emerge as a confluent monolayer after a period of time (e.g., 2 days, 3 days, 4 days, 5 days, 6 days, 1 week, 2 weeks, 3 weeks, 4 weeks, 5 weeks, 6 weeks, or more) in culture. Pathfinder cells in a monolayer can be harvested and optionally washed in a solution such as PBS (phosphate-buffered saline).

Suitable cell culture conditions are described below. In embodiments in which pathfinder cells are obtained from cell cultures, they may be of any passage number so long as they can still have one or more properties that confer therapeutic benefit. Thus, pathfinder cells of low (e.g., less than about 10 passages), medium (e.g., between to about 10 to about 20 passages), or high (e.g., more than about 20 passages (e.g., about 30, 40, 50, 60, 70, 80, 90, 100, or more than 100)) passage number may be used in various embodiments of the invention.

Cell Culture Conditions

In some embodiments, pathfinder cells are obtained from a cell culture that had been established from freshly isolated or stored materials. For example, cells may be cultured in a liquid medium that contains nutrients for the cells and are incubated in an environment where the temperature and/or gas composition is controlled. As will be appreciated by one of ordinary skill in the art, specific cell culture conditions may vary depending on the type of cells used. Cell culture conditions for pathfinder cells have been described. See, e.g., International Patent Publication WO2006/120476.

An exemplary suitable medium for culture of pathfinder cells is CMRL 1066 medium (Invitrogen) supplemented with fetal bovine serum (e.g., at 10%). In some embodiments, media is supplemented with glutamine or glutamine-containing mixtures such as GLUTAMAX™ (Invitrogen) and/or with antibiotics (e.g., amphotericin, penicillin, and/or streptomycin).

In some embodiments, cells are grown such they are attached on a surface. In some such embodiments, cells are grown as a monolayer on the surface. In some embodiments, cells are grown until they are confluent, i.e., until they cover the entire surface on which they are growing and there is nowhere else on the surface for cells to grow. In some embodiments, cells are grown until they are close to but not yet at confluence, i.e., until they cover most of the surface on which they are growing, but there is still some room for cells to grow. In some embodiments, cells are grown until they are approximately or more than 50%, 60%, 70%, 80%, 85%, 90%, 95%, 97%, 98%, 99%, or more confluent, wherein x % confluent is defined as coverage of approximately x % of the growing surface. In some embodiments, cells are grown until they are approximately 50-99% (e.g., 60-99%, 70-99%, 75-99%, 80-99%, 85-99%, 90-99%, or 95-99%) confluent.

In some embodiments, cells are grown on a substrate that may affect one or more properties of the cell, such as microvesicle production rate, cell proliferation rate, or miRNA expression pattern. In some embodiments, cells are grown on a nonwoven substrate such as a nonwoven fabric comprised of fibers. As used herein, the term “nonwoven fabric” includes, but is not limited to, bonded fabrics, formed fabrics, or engineered fabrics, that are manufactured by processes other than, weaving or knitting. In some embodiments, the term “nonwoven fabric” refers to a porous, textile-like material, usually in flat sheet form, composed primarily or entirely of fibers, such as staple fibers assembled in a web, sheet or batt. The structure of the nonwoven fabric may be based on the arrangement of, for example, staple fibers that are typically arranged more or less randomly. Nonwoven fabrics can be created by a variety of techniques known in the textile industry. Various methods may create carded, wet laid, melt blown, spunbonded, or air laid nonwovens. Exemplary methods and substrates are described in U.S. Application Publication No. 20100151575, the entire teachings of which are incorporated herein by reference. The density of the nonwoven fabrics may be varied depending upon the processing conditions. In one embodiment, the nonwoven fabrics have a density of about 60 mg/mL to about 350 mg/mL.

In some embodiments, the nonwoven substrates are biocompatible and/or bioabsorbable. Examples of suitable biocompatible, bioabsorbable polymers that could be used include polymers selected from the group consisting of aliphatic polyesters, poly(amino acids), copoly(ether-esters), polyalkylene oxalates, polyamides, poly(iminocarbonates), polyorthoesters, polyoxaesters, polyamidoesters, polyoxaesters containing amine groups, poly(anhydrides), polyphosphazenes, and blends thereof.

In some embodiments, the aliphatic polyesters are homopolymers and/or copolymers of monomers selected from the group consisting of lactide (which includes lactic acid, D-,L- and meso lactide), glycolide (including glycolic acid), epsilon-caprolactone, p-dioxanone (1,4-dioxan-2-one), trimethylene carbonate (1,3-dioxan-2-one), alkyl derivatives of trimethylene carbonate, delta-valerolactone, beta-butyrolactone, gamma-butyrolactone, epsilon-decalactone, hydroxybutyrate (repeating units), hydroxyvalerate (repeating units), 1,4-dioxepan-2-one (including its dimer 1,5,8,12-tetraoxacyclotetradecane-7,14-dione), 1,5-dioxepan-2-one, 6,6-dimethyl-1,4-dioxan-2-one and polymer blends thereof. In another embodiment, aliphatic polyesters which include, but are not limited to homopolymers and/or copolymers of lactide (which includes lactic acid, D-,L- and meso lactide), glycolide (including glycolic acid), epsilon-caprolactone, p-dioxanone (1,4-dioxan-2-one), trimethylene carbonate (1,3-dioxan-2-one) and combinations thereof.

In some embodiments, the aliphatic polyesters are homopolymers and/or copolymers of monomers selected from the group consisting of lactide (which includes lactic acid, D-,L- and meso lactide), glycolide (including glycolic acid), epsilon-caprolactone, p-dioxanone (1,4-dioxan-2-one), trimethylene carbonate (1,3-dioxan-2-one) and combinations thereof. In yet another embodiment, the aliphatic polyesters are homopolymers and/or copolymers of monomers selected from the group consisting of lactide (which includes lactic acid, D-,L- and meso lactide), glycolide (including glycolic acid), and p-dioxanone (1,4-dioxan-2-one) and combinations thereof. Non-limiting examples of suitable fabrics include those that comprise aliphatic polyester fibers, e.g., fibers that comprise homopolymers or copolymers of lactide (e.g., lactic acid D-. L- and meso lactide), glycolide (e.g., glycolic acid), epsilon-caprolactone, p-dioxanone (1,4-dioxan-2-one), trimethylene carbonate (1,3-dioxan-2-one), and combinations thereof. For example, suitable farbics may contain poly(glycolide-co-lactide) (PGA/PLA); poly(lactide-co-glycolide) (PLA/PGA); 1,3 propanediol (PDO), and/or blends thereof.

In some embodiments, cells are grown on a solid surface that has been textured in a particular way so as to confer special properties to the surface (e.g., repulsion or attraction of certain substances, reduced adsorption of proteins, etc.), which in turn may influence behavior of cells on such surfaces. For example, cells may be grown on a nano-textured surface (“nanosurface”). See, e.g., U.S. Pat. No. 7,597,950; Sun et al. (2009) “Combining nanosurface chemistry and microfluidics for molecular analysis and cell biology,” Analytica Chimica Acta, 650(1):98-105; the entire contents of each of which are herein incorporated by reference. Nanosurfaces and other textured surfaces may be generated, for example by any of a variety of methods known in the art, including sanding, chemical etching, sandblasting, and/or dewetting.

In some embodiments, cells are grown in suspension.

In some embodiments, cells are grown in a Matrigel free culture system in the presence of serum. Pathfinder cells have been shown to be able to grow in such systems. See, e.g., International Patent Publication WO 2006/120476.

In some embodiments, cells are subjected for at least part of the time to one or more conditions that enhance their growth, e.g., such that increased numbers of cells can be obtained from a culture. In some embodiments, enhancement of cell growth is accomplished by modulating accessibility of telomeres in cells. For example, cells may be first treated to reduce a telomeric attrition rate prior to cell culture. In some embodiments, to achieve reduced telomeric attrition rate, components of the telomere complex and/or factors that associate with it may be targeted by an agent that causes their downregulation or otherwise impairs their function. For example, cells may be exposed to inhibitory RNAs such as small inhibitory RNAs (siRNAs) to inhibit expression a gene encoding a protein or other gene product that forms part of the telomere complex or associates with the telomere complex. In some embodiments, RAF1 expression is inhibited with the use of a RAF1 specific siRNA. In some embodiments, STAU1 expression is inhibited with the use of a STAU1 specific siRNA. For a fuller discussion of methods for enhancing cell growth that may be used in accordance with the present invention, see, e.g., U.S. provisional application entitled “Enhanced Cell Growth by the Modulation of Telomore Accessibility” filed on even date, the entire contents of which are herein incorporated by reference.

Various growth media may be used to culture cells. Growth medium generally refers to any substance or preparation used for the cultivation of living cells. In some embodiments, the growth medium is renal growth medium. In some embodiment the growth medium is Dulbecco's Modification of Eagle's medium (DMEM). In some embodiments, cells are grown in media that does not contain serum. In some embodiments, cells are grown for at least a period of time in media that has been depleted of microvesicles from media components. For example, media containing fetal bovine serum may be depleted of bovine microvesicles. Alternatively or additionally, commercially available medium that is depleted of microvesicles (e.g., bovine microvesicles) is used.

In some embodiments, cells are grown at or about 37° C. In some embodiments, cells are grown in the presence of at or about 5% CO₂. In some embodiments, cells are grown under room air oxygen conditions. In some embodiments, cells are grown under conditions where the oxygen pressure is less than or equal to 5% O₂. In some embodiments, cells are grown in conditions of normal oxygen (e.g., about 5% O₂). In some embodiments, cells are grown in hypoxic conditions (e.g., low oxygen such as <5%, <4%, <3%, <2%, or <1% O₂).

In some embodiments, cells are grown under serum starvation conditions. As used herein, the term “serum starvation” includes, but is not limited to, serum depletion, serum-free medium or conditions. Various serum starvation conditions are known in the art and can be used to practice the present invention. In some embodiments, cells may be grown under serum starvation conditions for about 6, about 12, about 18, about 24, about 30, about 36, about 42, about 48 hours, or longer. In some embodiments, cells may be grown under conditions where the serum concentration is less than or equal to 10%, less than or equal to 9%, less than or equal to 8%, less than or equal to 7%, less than or equal to 6%, less than or equal to 5%, less than or equal to 4%, less than or equal to 3%, less than or equal to 2%, less than or equal to 1.5%, less than or equal to 1%, or less than or equal to 0.5%. In some embodiments, cells may be grown under conditions where the serum concentration is 0% (i.e., serum is absent). In some embodiments, cells may be grown under conditions where the serum concentration is decreased in a step-wise manner over time. For example, in some embodiments, cells may be grown under conditions where the serum concentration is between about 2% to about 11% (e.g., about 2%, 3%, 4%, 5%, 6%, 7%, 8%, 9%, 10%, or 11%) and is subsequently reduced in one or more steps to a serum concentration between about 0% to about 5% (e.g., about 0%, 0.5%, 1%, 1.5%, 2%, 3%, 4%, or 5%).

Cell Differentiation

In certain embodiments, pathfinder cells may differentiate into one or more cell types in the cell culture and the differentiated cell types are used in therapeutic applications in accordance with the invention. In some embodiments, pathfinder cells are differentiated fully to a defined cell type, including, but not limited to, pancreatic cells, neuronal cells, cardiovascular cells, epithelial cells, hepatocytes, muscle cells, retinal cells, hair follicle cells, and kidney cells.

In some embodiments, pathfinder cells are differentiated into an “intermediate” cell type that is less pluripotent than a pathfinder cell, yet has greater potency than at least most fully differentiated cells. For example, a pathfinder cell may be differentiated into one of several progenitor cells of the immune system (e.g., lymphoid progenitor cells or myeloid progenitor cells) which are less pluripotent than hematopoetic stem cells yet not fully differentiated.

In some embodiments, the cell type into which pathfinder cells are differentiated are tailored for the specific therapeutic indication for which the pathfinder cells are being used. For example, in some embodiments in which pathfinder cells are being used to stimulate repair in an individual with damage to the liver, pathfinder cells are differentiated into hepatocytes before administering to the individual.

Methods of differentiating pathfinder cells are known in the art and are described, for example, in WO 2009/136168. See also Example 11 in the present specification. Typically, pathfinder cells are cultured in a growth medium (“differentiation media”) distinct from the growth medium in which pathfinder cells were maintained before differentiation (“maintenance media”). Differentiation media may vary depending on intended cell type, and may, for example, have a composition known to be suitable for growth of a particular cell type. In some embodiments, differentiation media contains one or more elements (e.g., growth factors, serum, additives, etc.) that are not present in maintenance media. In some embodiments, differentiation media lack one or more elements (e.g., growth factors, serum, additives, etc.) that are present in maintenance media.

Alternative or additionally, other culture conditions are altered as compared to culture conditions used to isolate and/or maintain pathfinder cells. For example, pathfinder cells may be grown at a lower cell density than they are typically grown during maintenance. Without wishing to be bound by any particular theory, growth at lower cell densities affect the growth properties of the cells in a culture, because their ability to signal to one another is altered when the cell density in the plate is lower. As another non-limiting example, pathfinder cells may be grown on a different kind of surface (e.g., substrate) as was used to grown them for maintenance purposes. Such surfaces may help alter properties of the pathfinder cells and thereby help induce differentiation.

Biomarkers

In some embodiments, cells suitable for the present invention bear one or more characteristic biomarkers (e.g., protein or nucleic acid). As discussed in the Examples section, pathfinder cells may express one or more markers including, but not limited to, CD24, c-myc, HLA class 1 ABC, ICAM3, Nestin, Nanog, Oct4, Integrin α2+β1, Ngn3, and CD130. In some embodiments, pathfinder cells may express two, three, four, five, six, seven, eight, or all of the markers described herein. In some embodiments, pathfinder cells express one or more markers typically associated with stem cells, such as, for example, Nestin. In some embodiments, pathfinder cells suitable for the present invention express Oct 4, Nanog, and c-myc.

In some embodiments, pathfinder cells suitable for the present invention are characterized with the absence of certain biomarkers. In some embodiments, pathfinder cells suitable for the present invention do not express at least one marker selected from the group consisting of CD34, CD105, VCAM1, CXCR2, CD44, CD73, ICAM1, and NCAM. In some embodiments, pathfinder cells do not express two, three, four, five, six, or seven of the markers selected from the group consisting of CD34, CD105, VCAM1, CXCR2, CD44, CD73, ICAM1, and NCAM. In some embodiments, pathfinder cells suitable for the invention do not express any of the above biomarkers.

In some embodiments, pathfinder cells have the biomarker profile: CD24⁺, c-myc⁺, HLA class 1 ABC⁺, ICAM3⁺, Nestin⁺, Nanog⁺, Oct4⁺, Integrin α2+β1⁺, Ngn3⁺, CD130⁺, CD34⁻, CD105⁻, VCAM1⁻, CXCR2⁻, CD44⁻, CD73⁻, ICAM1⁻, and NCAM⁻.

In some embodiments, biomarkers may be used to isolate, enrich, and/or monitoring the growth of cells suitable for the present invention. For example, biomarkers may be used to prepare, isolate, or obtain a population of substantially homogeneous cells for therapeutic uses. As used herein, a population of substantially homogeneous cells refers to a population with at least 30% (e.g., at least 35%, 40%, 45%, 50%, 55%, 60%, 65%, 70%, 75%, 80%, 85%, 90%, 95%, or more) of the cells expressing one or more (e.g., two or more, three or more, four or more, five or more, six or more, seven or more, eight or more, or nine or more) identical biomarkers that are typically expressed on pathfinder cells. In some embodiments, suitable biomarkers include, but are not limited to, CD24, c-myc, HLA class 1 ABC, ICAM3, Nestin, Nanog, Oct4, Integrin a2+b1, Ngn3, and CD130. Additionally or alternatively, a population of substantially homogeneous cells refers to a population with at least 30% (e.g., at least 35%, 40%, 45%, 50%, 55%, 60%, 65%, 70%, 75%, 80%, 85%, 90%, 95%, or more) of the cells that do not express one or more (e.g., two or more, three or more, four or more, five or more, six or more, seven or more, eight or more, or nine or more) biomarkers that are typically not expressed on pathfinder cells. Such biomarkers include, but are not limited to, CD34, CD105, VCAM1, CXCR2, CD44, CD73, ICAM1, and NCAM.

Additionally or alternatively, nucleic acids such as the microRNAs described below may be used as biomarkers to isolate, enrich or monitoring the growth of pathfinder cells and to obtain a population of substantially homogeneous cells for therapeutic uses.

### II. MicroRNAs

In some embodiments, cells comprise one or more specific microRNAs. As used herein, “pathfinder cell microRNAs” include those microRNAs that are present in pathfinder cells derived from one or more tissues. “Pathfinder cell-specific microRNAs” are a subset of pathfinder cell microRNAs and include those microRNAs that are present in pathfinder cells but not other comparable cell types derived from the same tissue, such as differentiated cells.

The terms “pathfinder cell microRNAs” and “pathfinder cells-specific microRNAs” encompass microRNAs isolated or purified from pathfinder cells as well as microRNAs having the same sequence synthesized using recombinant or chemical techniques. For example, microRNA molecules may be generated by in vitro transcription of DNA sequences encoding the relevant molecule. Such DNA sequences may be incorporated into a wide variety of vectors with suitable RNA polymerase promoters such as T7, T3, or SP6. As used herein, the term “microRNAs (miRNAs)” refers to post-transcriptional regulators that typically bind to complementary sequences in the three prime untranslated regions (3′ UTRs) of target messenger RNA transcripts (mRNAs), usually resulting in gene silencing. Typically, miRNAs are short ribonucleic acid (RNA) molecules. For example, microRNAs may be approximately 18-25 nucleotides long (e.g., approximately 18, 19, 20, 21, 22, 23, 24 or 25 nucleotides long).

It is contemplated that pathfinder cell microRNAs, individually or in combination, may be used to induce or stimulate repair of tissues, cell growth, cell regeneration, remodeling, reconstruction, differentiation and/or transdifferentiation, among other functions. It is further contemplated that pathfinder cell microRNAs can stimulate repair of tissues that are damaged from trauma or other acute diseases, disorders, or conditions.

Table 1 shows exemplary microRNAs that are present in pathfinder cells. In some embodiments, it was found that SEQ ID NOs: 1 to 319 are present in pathfinder cells. Additional microRNAs identified according to the present invention are listed in Tables 6 to 10 and include SEQ ID NOs: 320 to 608. Table 1 below, and Tables 6-10 in the Examples, list exemplary miRNA sequences for each miRNA of interest; corresponding miRNA sequences in other species are publicly available (e.g., see http://diana.cslab.ece.ntua.gr/mirgen/). Some miRNA sequences are well conserved across species, including, but not limited to, Homo sapiens, Rattus norvegicus, Mus musculus, Danio rerio, and Gallus gallus, and some miRNA sequence variants exist even in the same species. In Table 1, asterisks (*) denote variants.

microRNAs useful in methods and compositions of the invention include those having nucleotide sequences identical to any of SEQ ID NOs: 1 to 319, as shown in Table 1. microRNAs useful in methods and compositions of the invention also include those having nucleotide sequences identical to any of SEQ ID NOs: 320 to 608, as shown in Tables 6-10. In some embodiments, microRNAs having at least 70%, 75%, 80%, 85%, 90%, 95%, or more nucleotide sequence identity with any of SEQ ID NOs: 1 to 610 (or any of SEQ ID NOs: 1 o 225) are used in methods and compositions of the invention.

TABLE 1
Exemplary microRNA sequences

| | | |
|---|---|---|
| | | SEQ ID |
| microRNA | Accession info, species and Sequence | NO. |

Expressed in hPDPCs, hKDPCs, and hLNDPCs

| | | |
|---|---|---|
| Let-7a | >hsa-let-7a MIMAT0000062 Homo sapiens let-7a | 1 |
| | UGAGGUAGUAGGUUGUAUAGUU | |
| | >hsa-let-7a* MIMAT0004481 Homo sapiens let-7a* | 2 |
| | CUAUACAAUCUACUGUCUUUC | |
| Let-7b | >hsa-let-7b MIMAT0000063 Homo sapiens let-7b | 3 |
| | UGAGGUAGUAGGUUGUGUGGUU | |
| | >hsa-let-7b* MIMAT0004482 Homo sapiens let-7b* | 4 |
| | CUAUACAACCUACUGCCUUCCC | |
| Let-7c | >hsa-let-7c MIMAT0000064 Homo sapiens let-7c | 5 |
| | UGAGGUAGUAGGUUGUAUGGUU | |
| | >hsa-let-7c* MIMAT0004483 Homo sapiens let-7c* | 6 |
| | UAGAGUUACACCCUGGGAGUUA | |
| Let-7d | >hsa-let-7d MIMAT0000065 Homo sapiens let-7d | 7 |
| | AGAGGUAGUAGGUUGCAUAGUU | |
| | >hsa-let-7d* MIMAT0004484 Homo sapiens let-7d* | 8 |
| | CUAUACGACCUGCUGCCUUUCU | |
| Let-7e | >hsa-let-7e MIMAT0000066 Homo sapiens let-7e | 9 |
| | UGAGGUAGGAGGUUGUAUAGUU | |
| | >hsa-let-7e* MIMAT0004485 Homo sapiens let-7e* | 10 |
| | CUAUACGGCCUCCUAGCUUUCC | |
| Let-7f | >hsa-let-7f MIMAT0000067 Homo sapiens let-7f | 11 |
| | UGAGGUAGUAGAUUGUAUAGUU | |
| Let-7g | >hsa-let-7g MIMAT0000414 Homo sapiens let-7g | 12 |
| | UGAGGUAGUAGUUUGUACAGUU | |
| | >hsa-let-7g* MIMAT0004584 Homo sapiens let-7g* | 13 |
| | CUGUACAGGCCACUGCCUUGC | |
| miR-9 | >hsa-miR-9 MIMAT0000441 Homo sapiens miR-9 | 14 |
| | UCUUUGGUUAUCUAGCUGUAUGA | |
| | >hsa-miR-9* MIMAT0000442 Homo sapiens miR-9* | 15 |
| | AUAAAGCUAGAUAACCGAAAGU | |
| miR-10a | >hsa-miR-10a MIMAT0000253 Homo sapiens miR-10a | 16 |
| | UACCCUGUAGAUCCGAAUUUGUG | |
| | >hsa-miR-10a* MIMAT0004555 Homo sapiens miR-10a* | 17 |
| | CAAAUUCGUAUCUAGGGGAAUA | |
| miR-10b | >hsa-miR-10b MIMAT0000254 Homo sapiens miR-10b | 18 |
| | UACCCUGUAGAACCGAAUUUGUG | |
| | >hsa-miR-10b* MIMAT0004556 Homo sapiens miR-10b* | 19 |
| | ACAGAUUCGAUUCUAGGGGAAU | |
| miR-15a | >hsa-miR-15a MIMAT0000068 Homo sapiens miR-15a | 20 |
| | UAGCAGCACAUAAUGGUUUGUG | |
| | >hsa-miR-15a* MIMAT0004488 Homo sapiens miR-15a* | 21 |
| | CAGGCCAUAUUGUGCUGCCUCA | |
| miR-15b | >hsa-miR-15b MIMAT0000417 Homo sapiens miR-15b | 22 |
| | UAGCAGCACAUCAUGGUUUACA | |
| | >hsa-miR-15b* MIMAT0004586 Homo sapiens miR-15b* | 23 |
| | CGAAUCAUUAUUUGCUGCUCUA | |
| miR-16 | >hsa-miR-16 MIMAT0000069 Homo sapiens miR-16 | 24 |
| | UAGCAGCACGUAAAUAUUGGCG | |
| miR-17 | >hsa-miR-17 MIMAT0000070 Homo sapiens miR-17 | 25 |
| | CAAAGUGCUUACAGUGCAGGUAG | |
| | >hsa-miR-17* MIMAT0000071 Homo sapiens miR-17* | 26 |
| | ACUGCAGUGAAGGCACUUGUAG | |
| miR-18a | >hsa-miR-18a MIMAT0000072 Homo sapiens miR-18a | 27 |
| | UAAGGUGCAUCUAGUGCAGAUAG | |
| | >hsa-miR-18a* MIMAT0002891 Homo sapiens miR-18a* | 28 |
| | ACUGCCCUAAGUGCUCCUUCUGG | |
| miR-19a | >hsa-miR-19a* MIMAT0004490 Homo sapiens miR-19a* | 29 |
| | AGUUUUGCAUAGUUGCACUACA | |
| | >hsa-miR-19a MIMAT0000073 Homo sapiens miR-19a | 30 |
| | UGUGCAAAUCUAUGCAAAACUGA | |
| miR-19b | >hsa-miR-19b MIMAT0000074 Homo sapiens miR-19b | 31 |
| | UGUGCAAAUCCAUGCAAAACUGA | |
| miR-20a | >hsa-miR-20a MIMAT0000075 Homo sapiens miR-20a | 32 |
| | UAAAGUGCUUAUAGUGCAGGUAG | |
| | >hsa-miR-20a* MIMAT0004493 Homo sapiens miR-20a* | 33 |
| | ACUGCAUUAUGAGCACUUAAAG | |
| miR-20b | >hsa-miR-20b MIMAT0001413 Homo sapiens miR-20b | 34 |
| | CAAAGUGCUCAUAGUGCAGGUAG | |
| | >hsa-miR-20b* MIMAT0004752 Homo sapiens miR-20b* | 35 |
| | ACUGUAGUAUGGGCACUUCCAG | |
| miR-21 | >hsa-miR-21 MIMAT0000076 Homo sapiens miR-21 | 36 |
| | UAGCUUAUCAGACUGAUGUUGA | |
| | >hsa-miR-21* MIMAT0004494 Homo sapiens miR-21* | 37 |
| | CAACACCAGUCGAUGGGCUGU | |
| miR-22 | >hsa-miR-22* MIMAT0004495 Homo sapiens miR-22* | 38 |
| | AGUUCUUCAGUGGCAAGCUUUA | |
| | >hsa-miR-22 MIMAT0000077 Homo sapiens miR-22 | 39 |
| | AAGCUGCCAGUUGAAGAACUGU | |
| miR-23b | >hsa-miR-23b* MIMAT0004587 Homo sapiens miR-23b* | 40 |
| | UGGGUUCCUGGCAUGCUGAUUU | |
| | >hsa-miR-23b MIMAT0000418 Homo sapiens miR-23b | 41 |
| | AUCACAUUGCCAGGGAUUACC | |
| miR-24 | >hsa-miR-24 MIMAT0000080 Homo sapiens miR-24 | 42 |
| | UGGCUCAGUUCAGCAGGAACAG | |
| miR-25 | >hsa-miR-25 MIMAT0000081 Homo sapiens miR-25 | 43 |
| | CAUUGCACUUGUCUCGGUCUGA | |
| miR-26a | >hsa-miR-26a MIMAT0000082 Homo sapiens miR-26a | 44 |
| | UUCAAGUAAUCCAGGAUAGGCU | |
| miR-26b | >hsa-miR-26b MIMAT0000083 Homo sapiens miR-26b | 45 |
| | UUCAAGUAAUUCAGGAUAGGU | |
| | >hsa-miR-26b* MIMAT0004500 Homo sapiens miR-26b* | 46 |
| | CCUGUUCUCCAUUACUUGGCUC | |
| miR-27a | >hsa-miR-27a* MIMAT0004501 Homo sapiens miR-27a* | 47 |
| | AGGGCUUAGCUGCUUGUGAGCA | |
| | >hsa-miR-27a MIMAT0000084 Homo sapiens miR-27a | 48 |
| | UUCACAGUGGCUAAGUUCCGC | |
| miR-27b | >hsa-miR-27b* MIMAT0004588 Homo sapiens miR-27b* | 49 |
| | AGAGCUUAGCUGAUUGGUGAAC | |
| miR-28-3p | >hsa-miR-28-3p MIMAT0004502 Homo sapiens miR-28-3p | 50 |
| | CACUAGAUUGUGAGCUCCUGGA | |
| miR-28-5p | >hsa-miR-28-5p MIMAT0000085 Homo sapiens miR-28-5p | 51 |
| | AAGGAGCUCACAGUCUAUUGAG | |
| miR-29a | >hsa-miR-29a MIMAT0000086 Homo sapiens miR-29a | 52 |
| | UAGCACCAUCUGAAAUCGGUUA | |
| miR-29c | >hsa-miR-29c* MIMAT0004673 Homo sapiens miR-29c* | 53 |
| | UGACCGAUUUCUCCUGGUGUUC | |
| | >hsa-miR-29c MIMAT0000681 Homo sapiens miR-29c | 54 |
| | UAGCACCAUUUGAAAUCGGUUA | |
| miR-30a | >hsa-miR-30a MIMAT0000087 Homo sapiens miR-30a | 55 |
| | UGUAAACAUCCUCGACUGGAAG | |
| | >hsa-miR-30a* MIMAT0000088 Homo sapiens miR-30a* | 56 |
| | CUUUCAGUCGGAUGUUUGCAGC | |
| miR-30b | >hsa-miR-30b MIMAT0000420 Homo sapiens miR-30b | 57 |
| | UGUAAACAUCCUACACUCAGCU | |
| | >hsa-miR-30b* MIMAT0004589 Homo sapiens miR-30b* | 58 |
| | CUGGGAGGUGGAUGUUUACUUC | |
| miR-30c | >hsa-miR-30c MIMAT0000244 Homo sapiens miR-30c | 59 |
| | UGUAAACAUCCUACACUCUCAGC | |
| miR-30d | >hsa-miR-30d MIMAT0000245 Homo sapiens miR-30d | 60 |
| | UGUAAACAUCCCCGACUGGAAG | |
| | >hsa-miR-30d* MIMAT0004551 Homo sapiens miR-30d* | 61 |
| | CUUUCAGUCAGAUGUUUGCUGC | |
| miR-30e | >hsa-miR-30e MIMAT0000692 Homo sapiens miR-30e | 62 |
| | UGUAAACAUCCUUGACUGGAAG | |
| | >hsa-miR-30e* MIMAT0000693 Homo sapiens miR-30e* | 63 |
| | CUUUCAGUCGGAUGUUUACAGC | |
| miR-32 | >hsa-miR-32 MIMAT0000090 Homo sapiens miR-32 | 64 |
| | UAUUGCACAUUACUAAGUUGCA | |
| | >hsa-miR-32* MIMAT0004505 Homo sapiens miR-32* | 65 |
| | CAAUUUAGUGUGUGUGAUAUUU | |
| miR-34a | >hsa-miR-34a MIMAT0000255 Homo sapiens miR-34a | 66 |
| | UGGCAGUGUCUUAGCUGGUUGU | |
| | >hsa-miR-34a* MIMAT0004557 Homo sapiens miR-34a* | 67 |
| | CAAUCAGCAAGUAUACUGCCCU | |
| miR-34c | >rno-miR-34c MIMAT0000814 Rattus norvegicus miR-34c | 68 |
| | AGGCAGUGUAGUUAGCUGAUUGC | |
| | >rno-miR-34c* MIMAT0004723 Rattus norvegicus miR-34c* | 69 |
| | AAUCACUAACCACACAGCCAGG | |
| miR-92a | >hsa-miR-92a MIMAT0000092 Homo sapiens miR-92a | 70 |
| | UAUUGCACUUGUCCCGGCCUGU | |
| miR-93 | >hsa-miR-93 MIMAT0000093 Homo sapiens miR-93 | 71 |
| | CAAAGUGCUGUUCGUGCAGGUAG | |
| | >hsa-miR-93* MIMAT0004509 Homo sapiens miR-93* | 72 |
| | ACUGCUGAGCUAGCACUUCCCG | |
| miR-98 | >hsa-miR-98 MIMAT0000096 Homo sapiens miR-98 | 73 |
| | UGAGGUAGUAAGUUGUAUUGUU | |
| miR-99a | >hsa-miR-99a MIMAT0000097 Homo sapiens miR-99a | 74 |
| | AACCCGUAGAUCCGAUCUUGUG | |
| miR-99b | >hsa-miR-99b MIMAT0000689 Homo sapiens miR-99b | 75 |
| | CACCCGUAGAACCGACCUUGCG | |
| | >hsa-miR-99b* MIMAT0004678 Homo sapiens miR-99b* | 76 |
| | CAAGCUCGUGUCUGUGGGUCCG | |
| miR-100 | >hsa-miR-100 MIMAT0000098 Homo sapiens miR-100 | 77 |
| | AACCCGUAGAUCCGAACUUGUG | |
| | >hsa-miR-100* MIMAT0004512 Homo sapiens miR-100* | 78 |
| | CAAGCUUGUAUCUAUAGGUAUG | |
| miR-101 | >hsa-miR-101* MIMAT0004513 Homo sapiens miR-101* | 79 |
| | CAGUUAUCACAGUGCUGAUGCU | |
| | >hsa-miR-101 MIMAT0000099 Homo sapiens miR-101 | 80 |
| | UACAGUACUGUGAUAACUGAA | |
| miR-103 | >hsa-miR-103 MIMAT0000101 Homo sapiens miR-103 | 81 |
| | AGCAGCAUUGUACAGGGCUAUGA | |
| miR-106a | >hsa-miR-106a MIMAT0000103 Homo sapiens miR-106a | 82 |
| | AAAAGUGCUUACAGUGCAGGUAG | |
| | >hsa-miR-106a* MIMAT0004517 Homo sapiens miR-106a* | 83 |
| | CUGCAAUGUAAGCACUUCUUAC | |
| miR-106b | >hsa-miR-106b MIMAT0000680 Homo sapiens miR-106b | 84 |
| | UAAAGUGCUGACAGUGCAGAU | |
| | >hsa-miR-106b* MIMAT0004672 Homo sapiens miR-106b* | 85 |
| | CCGCACUGUGGGUACUUGCUGC | |
| miR-107 | >hsa-miR-107 MIMAT0000104 Homo sapiens miR-107 | 86 |
| | AGCAGCAUUGUACAGGGCUAUCA | |
| miR-125a-3p | >hsa-miR-125a-3p MIMAT0004602 Homo sapiens miR-125a-3p | 87 |
| | ACAGGUGAGGUUCUUGGGAGCC | |
| miR-125a-5p | >hsa-miR-125a-5p MIMAT0000443 Homo sapiens miR-125a-5p | 88 |
| | UCCCUGAGACCCUUUAACCUGUGA | |
| miR-125b | >hsa-miR-125b MIMAT0000423 Homo sapiens miR-125b | 89 |
| | UCCCUGAGACCCUAACUUGUGA | |
| miR-126 | >hsa-miR-126* MIMAT0000444 Homo sapiens miR-126* | 90 |
| | CAUUAUUACUUUUGGUACGCG | |
| | >hsa-miR-126 MIMAT0000445 Homo sapiens miR-126 | 91 |
| | UCGUACCGUGAGUAAUAAUGCG | |
| miR-128 | >hsa-miR-128 MIMAT0000424 Homo sapiens miR-128 | 92 |
| | UCACAGUGAACCGGUCUCUUU | |
| miR-130a | >hsa-miR-130a* MIMAT0004593 Homo sapiens miR-130a* | 93 |
| | UUCACAUUGUGCUACUGUCUGC | |
| | >hsa-miR-130a MIMAT0000425 Homo sapiens miR-130a | 94 |
| | CAGUGCAAUGUUAAAAGGGCAU | |
| miR-130b | >hsa-miR-130b* MIMAT0004680 Homo sapiens miR-130b* | 95 |
| | ACUCUUUCCCUGUUGCACUAC | |
| | >hsa-miR-130b MIMAT0000691 Homo sapiens miR-130b | 96 |
| | CAGUGCAAUGAUGAAAGGGCAU | |
| miR-132 | >hsa-miR-132* MIMAT0004594 Homo sapiens miR-132* | 97 |
| | ACCGUGGCUUUCGAUUGUUACU | |
| | >hsa-miR-132 MIMAT0000426 Homo sapiens miR-132 | 98 |
| | UAACAGUCUACAGCCAUGGUCG | |
| miR-133a | >hsa-miR-133a MIMAT0000427 Homo sapiens miR-133a | 99 |
| | UUUGGUCCCCUUCAACCAGCUG | |
| miR-138 | >hsa-miR-138 MIMAT0000430 Homo sapiens miR-138 | 100 |
| | AGCUGGUGUUGUGAAUCAGGCCG | |
| miR-139-5p | >hsa-miR-139-5p MIMAT0000250 Homo sapiens miR-139-5p | 101 |
| | UCUACAGUGCACGUGUCUCCAG | |
| miR-140-3p | >hsa-miR-140-3p MIMAT0004597 Homo sapiens miR-140-3p | 102 |
| | UACCACAGGGUAGAACCACGG | |
| miR-140-5p | >hsa-miR-140-5p MIMAT0000431 Homo sapiens miR-140-5p | 103 |
| | CAGUGGUUUUACCCUAUGGUAG | |
| miR-143 | >hsa-miR-143* MIMAT0004599 Homo sapiens miR-143* | 104 |
| | GGUGCAGUGCUGCAUCUCUGGU | |
| | >hsa-miR-143 MIMAT0000435 Homo sapiens miR-143 | 105 |
| | UGAGAUGAAGCACUGUAGCUC | |
| miR-145 | >hsa-miR-145 MIMAT0000437 Homo sapiens miR-145 | 106 |
| | GUCCAGUUUUCCCAGGAAUCCCU | |
| miR-146a | >hsa-miR-146a MIMAT0000449 Homo sapiens miR-146a | 107 |
| | UGAGAACUGAAUUCCAUGGGUU | |
| | >hsa-miR-146a* MIMAT0004608 Homo sapiens miR-146a* | 108 |
| | CCUCUGAAAUUCAGUUCUUCAG | |
| miR-146b-5p | >hsa-miR-146b-5p MIMAT0002809 Homo sapiens miR-146b-5p | 109 |
| | UGAGAACUGAAUUCCAUAGGCU | |
| miR-148b | >hsa-miR-148b* MIMAT0004699 Homo sapiens miR-148b* | 110 |
| | AAGUUCUGUUAUACACUCAGGC | |
| | >hsa-miR-148b MIMAT0000759 Homo sapiens miR-148b | 111 |
| | UCAGUGCAUCACAGAACUUUGU | |
| miR-149 | >hsa-miR-149 MIMAT0000450 Homo sapiens miR-149 | 112 |
| | UCUGGCUCCGUGUCUUCACUCCC | |
| | >hsa-miR-149* MIMAT0004609 Homo sapiens miR-149* | 113 |
| | AGGGAGGGACGGGGGCUGUGC | |
| miR-150 | >hsa-miR-150 MIMAT0000451 Homo sapiens miR-150 | 114 |
| | UCUCCCAACCCUUGUACCAGUG | |
| | >hsa-miR-150* MIMAT0004610 Homo sapiens miR-150* | 115 |
| | CUGGUACAGGCCUGGGGGACAG | |
| miR-151-3p | >hsa-miR-151-3p MIMAT0000757 Homo sapiens miR-151-3p | 116 |
| | CUAGACUGAAGCUCCUUGAGG | |
| miR-152 | >hsa-miR-152 MIMAT0000438 Homo sapiens miR-152 | 117 |
| | UCAGUGCAUGACAGAACUUGG | |
| miR-155 | >hsa-miR-155 MIMAT0000646 Homo sapiens miR-155 | 118 |
| | UUAAUGCUAAUCGUGAUAGGGGU | |
| | >hsa-miR-155* MIMAT0004658 Homo sapiens miR-155* | 119 |
| | CUCCUACAUAUUAGCAUUAACA | |
| miR-181a | >hsa-miR-181a MIMAT0000256 Homo sapiens miR-181a | 120 |
| | AACAUUCAACGCUGUCGGUGAGU | |
| miR-181c | >hsa-miR-181c MIMAT0000258 Homo sapiens miR-181c | 121 |
| | AACAUUCAACCUGUCGGUGAGU | |
| | >hsa-miR-181c* MIMAT0004559 Homo sapiens miR-181c* | 122 |
| | AACCAUCGACCGUUGAGUGGAC | |
| miR-184 | >hsa-miR-184 MIMAT0000454 Homo sapiens miR-184 | 123 |
| | UGGACGGAGAACUGAUAAGGGU | |
| miR-185 | >hsa-miR-185 MIMAT0000455 Homo sapiens miR-185 | 124 |
| | UGGAGAGAAAGGCAGUUCCUGA | |
| | >hsa-miR-185* MIMAT0004611 Homo sapiens miR-185* | 125 |
| | AGGGGCUGGCUUUCCUCUGGUC | |
| miR-186 | >hsa-miR-186 MIMAT0000456 Homo sapiens miR-186 | 126 |
| | CAAAGAAUUCUCCUUUUGGGCU | |
| | >hsa-miR-186* MIMAT0004612 Homo sapiens miR-186* | 127 |
| | GCCCAAAGGUGAAGGG | |
| miR-188-5p | >hsa-miR-188-5p MIMAT0000457 Homo sapiens miR-188-5p | 128 |
| | CAUCCCUUGCAUGGUGGAGGG | |
| miR-190 | >hsa-miR-190 MIMAT0000458 Homo sapiens miR-190 | 129 |
| | UGAUAUGUUUGAUAUAUUAGGU | |
| miR-191 | >hsa-miR-191 MIMAT0000440 Homo sapiens miR-191 | 130 |
| | CAACGGAAUCCCAAAAGCAGCUG | |
| | >hsa-miR-191* MIMAT0001618 Homo sapiens miR-191* | 131 |
| | GCUGCGCUUGGAUUUCGUCCCC | |
| miR-192 | >hsa-miR-192 MIMAT0000222 Homo sapiens miR-192 | 132 |
| | CUGACCUAUGAAUUGACAGCC | |
| | >hsa-miR-192* MIMAT0004543 Homo sapiens miR-192* | 133 |
| | CUGCCAAUUCCAUAGGUCACAG | |
| miR-193a-3p | >hsa-miR-193a-3p MIMAT0000459 Homo sapiens miR-193a-3p | 134 |
| | AACUGGCCUACAAAGUCCCAGU | |
| miR-193a-5 | >hsa-miR-193a-5p MIMAT0004614 Homo sapiens miR-193a-5p | 135 |
| | UGGGUCUUUGCGGGCGAGAUGA | |
| miR-193b | >hsa-miR-193b* MIMAT0004767 Homo sapiens miR-193b* | 136 |
| | CGGGGUUUUGAGGGCGAGAUGA | |
| | >hsa-miR-193b MIMAT0002819 Homo sapiens miR-193b | 137 |
| | AACUGGCCCUCAAAGUCCCGCU | |
| miR-194 | >hsa-miR-194 MIMAT0000460 Homo sapiens miR-194 | 138 |
| | UGUAACAGCAACUCCAUGUGGA | |
| miR-195 | >hsa-miR-195 MIMAT0000461 Homo sapiens miR-195 | 139 |
| | UAGCAGCACAGAAAUAUUGGC | |
| | >hsa-miR-195* MIMAT0004615 Homo sapiens miR-195* | 140 |
| | CCAAUAUUGGCUGUGCUGCUCC | |
| miR-197 | >hsa-miR-197 MIMAT0000227 Homo sapiens miR-197 | 141 |
| | UUCACCACCUUCUCCACCCAGC | |
| miR-199a-3p | >hsa-miR-199a-3p MIMAT0000232 Homo sapiens miR-199a-3p | 142 |
| | ACAGUAGUCUGCACAUUGGUUA | |
| miR-199a-5p | >hsa-miR-199a-5p MIMAT0000231 Homo sapiens miR-199a-5p | 143 |
| | CCCAGUGUUCAGACUACCUGUUC | |
| miR-199b-5p | >hsa-miR-199b-5p MIMAT0000263 Homo sapiens miR-199b-5p | 144 |
| | CCCAGUGUUUAGACUAUCUGUUC | |
| miR-200b | >hsa-miR-200b* MIMAT0004571 Homo sapiens miR-200b* | 145 |
| | CAUCUUACUGGGCAGCAUUGGA | |
| | >hsa-miR-200b MIMAT0000318 Homo sapiens miR-200b | 146 |
| | UAAUACUGCCUGGUAAUGAUGA | |
| miR-200c | >hsa-miR-200c* MIMAT0004657 Homo sapiens miR-200c* | 147 |
| | CGUCUUACCCAGCAGUGUUUGG | |
| | >hsa-miR-200c MIMAT0000617 Homo sapiens miR-200c | 148 |
| | UAAUACUGCCGGGUAAUGAUGGA | |
| miR-206 | >hsa-miR-206 MIMAT0000462 Homo sapiens miR-206 | 149 |
| | UGGAAUGUAAGGAAGUGUGUGG | |
| miR-210 | >hsa-miR-210 MIMAT0000267 Homo sapiens miR-210 | 150 |
| | CUGUGCGUGUGACAGCGGCUGA | |
| miR-212 | >hsa-miR-212 MIMAT0000269 Homo sapiens miR-212 | 151 |
| | UAACAGUCUCCAGUCACGGCC | |
| miR-214 | >hsa-miR-214* MIMAT0004564 Homo sapiens miR-214* | 152 |
| | UGCCUGUCUACACUUGCUGUGC | |
| | >hsa-miR-214 MIMAT0000271 Homo sapiens miR-214 | 153 |
| | ACAGCAGGCACAGACAGGCAGU | |
| miR-218 | >hsa-miR-218 MIMAT0000275 Homo sapiens miR-218 | 154 |
| | UUGUGCUUGAUCUAACCAUGU | |
| miR-221 | >hsa-miR-221* MIMAT0004568 Homo sapiens miR-221* | 155 |
| | ACCUGGCAUACAAUGUAGAUUU | |
| | >hsa-miR-221 MIMAT0000278 Homo sapiens miR-221 | 156 |
| | AGCUACAUUGUCUGCUGGGUUUC | |
| miR-222 | >hsa-miR-222* MIMAT0004569 Homo sapiens miR-222* | 157 |
| | CUCAGUAGCCAGUGUAGAUCCU | |
| | >hsa-miR-222 MIMAT0000279 Homo sapiens miR-222 | 158 |
| | AGCUACAUCUGGCUACUGGGU | |
| miR-223 | >hsa-miR-223* MIMAT0004570 Homo sapiens miR-223* | 159 |
| | CGUGUAUUUGACAAGCUGAGUU | |
| | >hsa-miR-223 MIMAT0000280 Homo sapiens miR-223 | 160 |
| | UGUCAGUUUGUCAAAUACCCCA | |
| miR-224 | >hsa-miR-224 MIMAT0000281 Homo sapiens miR-224 | 161 |
| | CAAGUCACUAGUGGUUCCGUU | |
| | >hsa-miR-224* MIMAT0009198 Homo sapiens miR-224* | 162 |
| | AAAAUGGUGCCCUAGUGACUACA | |
| miR-296-5p | >hsa-miR-296-5p MIMAT0000690 Homo sapiens miR-296-5p | 163 |
| | AGGGCCCCCCCUCAAUCCUGU | |
| miR-301a | >hsa-miR-301a MIMAT0000688 Homo sapiens miR-301a | 164 |
| | CAGUGCAAUAGUAUUGUCAAAGC | |
| miR-301b | >hsa-miR-301b MIMAT0004958 Homo sapiens miR-301b | 165 |
| | CAGUGCAAUGAUAUUGUCAAAGC | |
| miR-320 | >mmu-miR-320 MIMAT0000666 Mus musculus miR-320 | 166 |
| | AAAAGCUGGGUUGAGAGGGCGA | |
| miR-323-3p | >hsa-miR-323-3p MIMAT0000755 Homo sapiens miR-323-3p | 167 |
| | CACAUUACACGGUCGACCUCU | |
| miR-324-3p | >hsa-miR-324-3p MIMAT0000762 Homo sapiens miR-324-3p | 168 |
| | ACUGCCCCAGGUGCUGCUGG | |
| miR-324-5p | >hsa-miR-324-5p MIMAT0000761 Homo sapiens miR-324-5p | 169 |
| | CGCAUCCCCUAGGGCAUUGGUGU | |
| miR-328 | >hsa-miR-328 MIMAT0000752 Homo sapiens miR-328 | 170 |
| | CUGGCCCUCUCUGCCCUUCCGU | |
| miR-331-3p | >hsa-miR-331-3p MIMAT0000760 Homo sapiens miR-331-3p | 171 |
| | GCCCCUGGGCCUAUCCUAGAA | |
| miR-331-5p | >hsa-miR-331-5p MIMAT0004700 Homo sapiens miR-331-5p | 172 |
| | CUAGGUAUGGUCCCAGGGAUCC | |
| miR-339-3p | >hsa-miR-339-3p MIMAT0004702 Homo sapiens miR-339-3p | 173 |
| | UGAGCGCCUCGACGACAGAGCCG | |
| miR-339-5P | >hsa-miR-339-5p MIMAT0000764 Homo sapiens miR-339-5p | 174 |
| | UCCCUGUCCUCCAGGAGCUCACG | |
| miR-361-5p | >hsa-miR-361-5p MIMAT0000703 Homo sapiens miR-361-5p | 175 |
| | UUAUCAGAAUCUCCAGGGGUAC | |
| miR-365 | >mmu-miR-365 MIMAT0000711 Mus musculus miR-365 | 176 |
| | UAAUGCCCCUAAAAAUCCUUAU | |
| | >hsa-miR-365* MIMAT0009199 Homo sapiens miR-365* | 177 |
| | AGGGACUUUCAGGGGCAGCUGU | |
| miR-367 | >hsa-miR-367* MIMAT0004686 Homo sapiens miR-367* | 178 |
| | ACUGUUGCUAAUAUGCAACUCU | |
| | >hsa-miR-367 MIMAT0000719 Homo sapiens miR-367 | 179 |
| | AAUUGCACUUUAGCAAUGGUGA | |
| miR-374b | >hsa-miR-374b MIMAT0004955 Homo sapiens miR-374b | 180 |
| | AUAUAAUACAACCUGCUAAGUG | |
| | >hsa-miR-374b* MIMAT0004956 Homo sapiens miR-374b* | 181 |
| | CUUAGCAGGUUGUAUUAUCAUU | |
| miR-378 | >hsa-miR-378* MIMAT0000731 Homo sapiens miR-378* | 182 |
| | CUCCUGACUCCAGGUCCUGUGU | |
| | >hsa-miR-378 MIMAT0000732 Homo sapiens miR-378 | 183 |
| | ACUGGACUUGGAGUCAGAAGG | |
| miR-422a | >hsa-miR-422a MIMAT0001339 Homo sapiens miR-422a | 184 |
| | ACUGGACUUAGGGUCAGAAGGC | |
| miR-423-5p | >hsa-miR-423-5p MIMAT0004748 Homo sapiens miR-423-5p | 185 |
| | UGAGGGGCAGAGAGCGAGACUUU | |
| miR-425 | >hsa-miR-425 MIMAT0003393 Homo sapiens miR-425 | 186 |
| | AAUGACACGAUCACUCCCGUUGA | |
| | >hsa-miR-425* MIMAT0001343 Homo sapiens miR-425* | 187 |
| | AUCGGGAAUGUCGUGUCCGCCC | |
| miR-449b | >hsa-miR-449b MIMAT0003327 Homo sapiens miR-449b | 188 |
| | AGGCAGUGUAUUGUUAGCUGGC | |
| | >hsa-miR-449b* MIMAT0009203 Homo sapiens miR-449b* | 189 |
| | CAGCCACAACUACCCUGCCACU | |
| miR-450a | >hsa-miR-450a MIMAT0001545 Homo sapiens miR-450a | 190 |
| | UUUUGCGAUGUGUUCCUAAUAU | |
| miR-452 | >hsa-miR-452 MIMAT0001635 Homo sapiens miR-452 | 191 |
| | AACUGUUUGCAGAGGAAACUGA | |
| | >hsa-miR-452* MIMAT0001636 Homo sapiens miR-452* | 192 |
| | CUCAUCUGCAAAGAAGUAAGUG | |
| miR-483-5p | >hsa-miR-483-5p MIMAT0004761 Homo sapiens miR-483-5p | 193 |
| | AAGACGGGAGGAAAGAAGGGAG | |
| miR-484 | >hsa-miR-484 MIMAT0002174 Homo sapiens miR-484 | 194 |
| | UCAGGCUCAGUCCCCUCCCGAU | |
| miR-485-3p | >hsa-miR-485-3p MIMAT0002176 Homo sapiens miR-485-3p | 195 |
| | GUCAUACACGGCUCUCCUCUCU | |
| miR-489 | >hsa-miR-489 MIMAT0002805 Homo sapiens miR-489 | 196 |
| | GUGACAUCACAUAUACGGCAGC | |
| miR-494 | >hsa-miR-494 MIMAT0002816 Homo sapiens miR-494 | 197 |
| | UGAAACAUACACGGGAAACCUC | |
| miR-497 | >hsa-miR-497 MIMAT0002820 Homo sapiens miR-497 | 198 |
| | CAGCAGCACACUGUGGUUUGU | |
| | >hsa-miR-497* MIMAT0004768 Homo sapiens miR-497* | 199 |
| | CAAACCACACUGUGGUGUUAGA | |
| miR-509-3p | >hsa-miR-509-3p MIMAT0002881 Homo sapiens miR-509-3p | 200 |
| | UGAUUGGUACGUCUGUGGGUAG | |
| miR-516a-3p | >hsa-miR-516a-3p MIMAT0006778 Homo sapiens miR-516a-3p | 201 |
| | UGCUUCCUUUCAGAGGGU | |
| miR-532-3p | >hsa-miR-532-3p MIMAT0004780 Homo sapiens miR-532-3p | 202 |
| | CCUCCCACACCCAAGGCUUGCA | |
| miR-532-5p | >hsa-miR-532-5p MIMAT0002888 Homo sapiens miR-532-5p | 203 |
| | CAUGCCUUGAGUGUAGGACCGU | |
| miR-542-3p | >hsa-miR-532-3p MIMAT0004780 Homo sapiens miR-532-3p | 204 |
| | CCUCCCACACCCAAGGCUUGCA | |
| miR-550 | >hsa-miR-550 MIMAT0004800 Homo sapiens miR-550 | 205 |
| | AGUGCCUGAGGGAGUAAGAGCCC | |
| | >hsa-miR-550* MIMAT0003257 Homo sapiens miR-550* | 206 |
| | UGUCUUACUCCCUCAGGCACAU | |
| miR-564 | >hsa-miR-564 MIMAT0003228 Homo sapiens miR-564 | 207 |
| | AGGCACGGUGUCAGCAGGC | |
| miR-572 | >hsa-miR-572 MIMAT0003237 Homo sapiens miR-572 | 208 |
| | GUCCGCUCGGCGGUGGCCCA | |
| miR-574-3p | >hsa-miR-574-3p MIMAT0003239 Homo sapiens miR-574-3p | 209 |
| | CACGCUCAUGCACACACCCACA | |
| miR-596 | >hsa-miR-596 MIMAT0003264 Homo sapiens miR-596 | 210 |
| | AAGCCUGCCCGGCUCCUCGGG | |
| miR-610 | >hsa-miR-610 MIMAT0003278 Homo sapiens miR-610 | 211 |
| | UGAGCUAAAUGUGUGCUGGGA | |
| miR-629 | >hsa-miR-629 MIMAT0004810 Homo sapiens miR-629 | 212 |
| | UGGGUUUACGUUGGGAGAACU | |
| | >hsa-miR-629* MIMAT0003298 Homo sapiens miR-629* | 213 |
| | GUUCUCCCAACGUAAGCCCAGC | |
| miR-639 | >hsa-miR-639 MIMAT0003309 Homo sapiens miR-639 | 214 |
| | AUCGCUGCGGUUGCGAGCGCUGU | |
| miR-652 | >hsa-miR-652 MIMAT0003322 Homo sapiens miR-652 | 215 |
| | AAUGGCGCCACUAGGGUUGUG | |
| miR-661 | >hsa-miR-661 MIMAT0003324 Homo sapiens miR-661 | 216 |
| | UGCCUGGGUCUCUGGCCUGCGCGU | |
| miR-671-3p | >hsa-miR-671-3p MIMAT0004819 Homo sapiens miR-671-3p | 217 |
| | UCCGGUUCUCAGGGCUCCACC | |
| miR-708 | >hsa-miR-708 MIMAT0004926 Homo sapiens miR-708 | 218 |
| | AAGGAGCUUACAAUCUAGCUGGG | |
| | >hsa-miR-708* MIMAT0004927 Homo sapiens miR-708* | 219 |
| | CAACUAGACUGUGAGCUUCUAG | |
| miR-744 | >hsa-miR-744 MIMAT0004945 Homo sapiens miR-744 | 609 |
| | UGCGGGGCUAGGGCUAACAGCA | |
| | >hsa-miR-744* MIMAT0004946 Homo sapiens miR-744* | 610 |
| | CUGUUGCCACUAACCUCAACCU | |
| miR-760 | >hsa-miR-760 MIMAT0004957 Homo sapiens miR-760 | 220 |
| | CGGCUCUGGGUCUGUGGGGA | |
| miR-770-5p | >hsa-miR-770-5p MIMAT0003948 Homo sapiens miR-770-5p | 221 |
| | UCCAGUACCACGUGUCAGGGCCA | |
| miR-875-5p | >hsa-miR-875-5p MIMAT0004922 Homo sapiens miR-875-5p | 222 |
| | UAUACCUCAGUUUUAUCAGGUG | |
| miR-877 | >hsa-miR-877 MIMAT0004949 Homo sapiens miR-877 | 223 |
| | GUAGAGGAGAUGGCGCAGGG | |
| | >hsa-miR-877* MIMAT0004950 Homo sapiens miR-877* | 224 |
| | UCCUCUUCUCCCUCCUCCCAG | |
| miR-939 | >hsa-miR-939 MIMAT0004982 Homo sapiens miR-939 | 225 |
| | UGGGGAGCUGAGGCUCUGGGGGUG | |

Expressed in hKPDCs and hLNDPCs but not in hPDPCs (18 miRNAs)

| | | |
|---|---|---|
| ma-7 | >hsa-miR-7 MIMAT0000252 Homo sapiens miR-7 | 226 |
| | UGGAAGACUAGUGAUUUUGUUGU | |
| miR-18b | >hsa-miR-18b MIMAT0001412 Homo sapiens miR-18b | 227 |
| | UAAGGUGCAUCUAGUGCAGUUAG | |
| | >hsa-miR-18b* MIMAT0004751 Homo sapiens miR-18b* | 228 |
| | UGCCCUAAAUGCCCCUUCUGGC | |
| miR-135b | >hsa-miR-135b MIMAT0000758 Homo sapiens miR-135b | 229 |
| | UAUGGCUUUUCAUUCCUAUGUGA | |
| | >hsa-miR-135b* MIMAT0004698 Homo sapiens miR-135b* | 230 |
| | AUGUAGGGCUAAAAGCCAUGGG | |
| miR142-3p | >hsa-miR-142-3p MIMAT0000434 Homo sapiens miR-142-3p | 231 |
| | UGUAGUGUUUCCUACUUUAUGGA | |
| miR-142-5p | >hsa-miR-142-5p MIMAT0000433 Homo sapiens miR-142-5p | 232 |
| | CAUAAAGUAGAAAGCACUACU | |
| miR-147b | >hsa-miR-147b MIMAT0004928 Homo sapiens miR-147b | 233 |
| | GUGUGCGGAAAUGCUUCUGCUA | |
| miR-187 | >hsa-miR-187 MIMAT0000262 Homo sapiens miR-187 | 234 |
| | UCGUGUCUUGUGUUGCAGCCGG | |
| | >hsa-miR-187* MIMAT0004561 Homo sapiens miR-187* | 235 |
| | GGCUACAACACAGGACCCGGGC | |
| miR-196b | >hsa-miR-196b MIMAT0001080 Homo sapiens miR-196b | 236 |
| | UAGGUAGUUUCCUGUUGUUGGG | |
| | >hsa-miR-196b* MIMAT0009201 Homo sapiens miR-196b* | 237 |
| | UCGACAGCACGACACUGCCUUC | |
| miR-204 | >hsa-miR-204 MIMAT0000265 Homo sapiens miR-204 | 238 |
| | UUCCCUUUGUCAUCCUAUGCCU | |
| miR-216a | >hsa-miR-216a MIMAT0000273 Homo sapiens miR-216a | 239 |
| | UAAUCUCAGCUGGCAACUGUGA | |
| miR-296-3p | >hsa-miR-296-3p MIMAT0004679 Homo sapiens miR-296-3p | 240 |
| | GAGGGUUGGGUGGAGGCUCUCC | |

Expressed in hPDPCs and hLNDPCs

| | | |
|---|---|---|
| miR-200a | >hsa-miR-200a MIMAT0000682 Homo sapiens miR-200a | 241 |
| | UAACACUGUCUGGUAACGAUGU | |
| | >hsa-miR-200a* MIMAT0001620 Homo sapiens miR-200a* | 242 |
| | CAUCUUACCGGACAGUGCUGGA | |
| miR-361-3p | >hsa-miR-361-3p MIMAT0004682 Homo sapiens miR-361-3p | 243 |
| | UCCCCCAGGUGUGAUUCUGAUUU | |
| miR-374a | >hsa-miR-374a MIMAT0000727 Homo sapiens miR-374a | 244 |
| | UUAUAAUACAACCUGAUAAGUG | |
| | >hsa-miR-374a* MIMAT0004688 Homo sapiens miR-374a* | 245 |
| | CUUAUCAGAUUGUAUUGUAAUU | |
| miR455 | >hsa-miR-455-5p MIMAT0003150 Homo sapiens miR-455-5p | 246 |
| | UAUGUGCCUUUGGACUACAUC | |
| | >hsa-miR-455-3p MIMAT0004784 Homo sapiens miR-455-3p | 247 |
| | GCAGUCCAUGGGCAUAUACAC | |

Expressed in hPDPCs and hKDPCs

| | | |
|---|---|---|
| miR-129-3p | hsa-miR-129-3p MIMAT0004605 Homo sapiens miR-129-3p | 248 |
| | AAGCCCUUACCCCAAAAAGCAU | |
| miR-133b | >hsa-miR-133b MIMAT0000770 Homo sapiens miR-133b | 249 |
| | UUUGGUCCCCUUCAACCAGCUA | |
| miR-190b | >hsa-miR-190b MIMAT0004929 Homo sapiens miR-190b | 250 |
| | UGAUAUGUUUGAUAUUGGGUU | |
| miR-342-3p | >hsa-miR-342-3p MIMAT0000753 Homo sapiens miR-342-3p | 251 |
| | UCUCACACAGAAAUCGCACCCGU | |
| miR-513a-3p | >hsa-miR-513a-3p MIMAT0004777 Homo sapiens miR-513a-3p | 252 |
| | UAAAUUUCACCUUUCUGAGAAGG | |
| miR-513a-5p | >has-miR-513a-5p MIMAT0002877 Homo sapiens miR-513a-5p | 253 |
| | UUCACAGGGAGGUGUCAU | |
| miR-571 | >hsa-miR-571 MIMAT0003236 Homo sapiens miR-571 | 254 |
| | UGAGUUGGCCAUCUGAGUGAG | |
| miR-592 | >hsa-miR-592 MIMAT0003260 Homo sapiens miR-592 | 255 |
| | UUGUGUCAAUAUGCGAUGAUGU | |
| miR-638 | >hsa-miR-638 MIMAT0003308 Homo sapiens miR-638 | 256 |
| | AGGGAUCGCGGGCGGGUGGCGGCCU | |
| miR-657 | >hsa-miR-657 MIMAT0003335 Homo sapiens miR-657 | 257 |
| | GGCAGGUUCUCACCCUCUCUAGG | |
| miR-659 | >hsa-miR-659 MIMAT0003337 Homo sapiens miR-659 | 258 |
| | CUUGGUUCAGGGAGGGUCCCCA | |
| miR-766 | >hsa-miR-766 MIMAT0003888 Homo sapiens miR-766 | 259 |
| | ACUCCAGCCCCACAGCCUCAGC | |

Expressed in hPDPCs

| | | |
|---|---|---|
| miR-122 | >hsa-miR-122 MIMAT0000421 Homo sapiens miR-122 | 260 |
| | UGGAGUGUGACAAUGGUGUUUG | |
| miR-127 | >rno-miR-127 MIMAT0000833 Rattus norvegicus miR-127 | 261 |
| | UCGGAUCCGUCUGAGCUUGGCU | |
| miR-134 | >hsa-miR-134 MIMAT0000447 Homo sapiens miR-134 | 262 |
| | UGUGACUGGUUGACCAGAGGGG | |
| miR-137 | >hsa-miR-137 MIMAT0000429 Homo sapiens miR-137 | 263 |
| | UUAUUGCUUAAGAAUACGCGUAG | |
| miR-148a | >hsa-miR-148a* MIMAT0004549 Homo sapiens miR-148a* | 264 |
| | AAAGUUCUGAGACACUCCGACU | |
| miR-299-5p | >hsa-miR-299-5p MIMAT0002890 Homo sapiens miR-299-5p | 265 |
| | UGGUUUACCGUCCCACAUACAU | |
| miR-302b | >hsa-miR-302b* MIMAT0000714 Homo sapiens miR-302b* | 266 |
| | ACUUUAACAUGGAAGUGCUUUC | |
| miR-337-5p | >hsa-miR-337-5p MIMAT0004695 Homo sapiens miR-337-5p | 267 |
| | GAACGGCUUCAUACAGGAGUU | |
| miR-340 | >hsa-miR-340 MIMAT0004692 Homo sapiens miR-340 | 268 |
| | UUAUAAAGCAAUGAGACUGAUU | |
| | >hsa-miR-340* MIMAT0000750 Homo sapiens miR-340* | 269 |
| | UCCGUCUCAGUUACUUUAUAGC | |
| miR-345 | >hsa-miR-345 MIMAT0000772 Homo sapiens miR-345 | 270 |
| | GCUGACUCCUAGUCCAGGGCUC | |
| miR-362-5p | >hsa-miR-362-5p MIMAT0000705 Homo sapiens miR-362-5p | 271 |
| | AAUCCUUGGAACCUAGGUGUGAGU | |
| miR-369-5p | >hsa-miR-369-5p MIMAT0001621 Homo sapiens miR-369-5p | 272 |
| | AGAUCGACCGUGUUAUAUUCGC | |
| miR-370 | >hsa-miR-370 MIMAT0000722 Homo sapiens miR-370 | 273 |
| | GCCUGCUGGGGUGGAACCUGGU | |
| miR-376a | >hsa-miR-376a MIMAT0000729 Homo sapiens miR-376a | 274 |
| | AUCAUAGAGGAAAAUCCACGU | |
| | >hsa-miR-376a* MIMAT0003386 Homo sapiens miR-376a* | 275 |
| | GUAGAUUCUCCUUCUAUGAGUA | |
| miR-376b | >mmu-miR-376b MIMAT0001092 | 276 |
| | AUCAUAGAGGAACAUCCACUU | |
| | >rno-miR-376b-5p MIMAT0003195 | 277 |
| | GUGGAUAUUCCUUCUAUGGUUA | |
| miR-376c | >rno-miR-376c MIMAT0003194 | 278 |
| | AACAUAGAGGAAAUUUCACGU | |
| miR-379 | >hsa-miR-379 MIMAT0000733 Homo sapiens miR-379 | 279 |
| | UGGUAGACUAUGGAACGUAGG | |
| | >hsa-miR-379* MIMAT0004690 Homo sapiens miR-379* | 280 |
| | UAUGUAACAUGGUCCACUAACU | |
| miR-409-3p | >hsa-miR-409-3p MIMAT0001639 Homo sapiens miR-409-3p | 281 |
| | GAAUGUUGCUCGGUGAACCCCU | |
| miR-411 | >hsa-miR-411 MIMAT0003329 Homo sapiens miR-411 | 282 |
| | UAGUAGACCGUAUAGCGUACG | |
| | >hsa-miR-411* MIMAT0004813 Homo sapiens miR-411* | 283 |
| | UAUGUAACACGGUCCACUAACC | |
| miR-424 | >hsa-miR-424 MIMAT0001341 Homo sapiens miR-424 | 284 |
| | CAGCAGCAAUUCAUGUUUUGAA | |
| miR-432 | >hsa-miR-432 MIMAT0002814 Homo sapiens miR-432 | 285 |
| | UCUUGGAGUAGGUCAUUGGGUGG | |
| | >hsa-miR-432* MIMAT0002815 Homo sapiens miR-432* | 286 |
| | CUGGAUGGCUCCUCCAUGUCU | |
| miR-433 | >hsa-miR-433 MIMAT0001627 Homo sapiens miR-433 | 287 |
| | AUCAUGAUGGGCUCCUCGGUGU | |
| miR-450b-5p | >hsa-miR-450b-5p MIMAT0004909 Homo sapiens miR-450b-5p | 288 |
| | UUUUGCAAUAUGUUCCUGAAUA | |
| | >hsa-miR-450b-3p MIMAT0004910 Homo sapiens miR-450b-3p | 289 |
| | UUGGGAUCAUUUUGCAUCCAUA | |
| miR-454 | >hsa-miR-454 MIMAT0003885 Homo sapiens miR-454 | 290 |
| | UAGUGCAAUAUUGCUUAUAGGGU | |
| | >hsa-miR-454* MIMAT0003884 Homo sapiens miR-454* | 291 |
| | ACCCUAUCAAUAUUGUCUCUGC | |
| miR-455-3p | >hsa-miR-455-3p MIMAT0004784 Homo sapiens miR-455-3p | 292 |
| | GCAGUCCAUGGGCAUAUACAC | |
| miR-486 | >mmu-miR-486 MIMAT0003130 Mus musculus miR-486 | 293 |
| | UCCUGUACUGAGCUGCCCCGAG | |
| miR-487b | >hsa-miR-487b MIMAT0003180 Homo sapiens miR-487b | 294 |
| | AAUCGUACAGGGUCAUCCACUU | |
| miR-490-3p | >hsa-miR-490-3p MIMAT0002806 Homo sapiens miR-490-3p | 295 |
| | CAACCUGGAGGACUCCAUGCUG | |
| miR-491-5p | >hsa-miR-491-5p MIMAT0002807 Homo sapiens miR-491-5p | 296 |
| | AGUGGGGAACCCUUCCAUGAGG | |
| miR-493 | >hsa-miR-493 MIMAT0003161 Homo sapiens miR-493 | 297 |
| | UGAAGGUCUACUGUGUGCCAGG | |
| | >hsa-miR-493* MIMAT0002813 Homo sapiens miR-493* | 298 |
| | UUGUACAUGGUAGGCUUUCAUU | |
| miR-495 | >hsa-miR-495 MIMAT0002817 Homo sapiens miR-495 | 299 |
| | AAACAAACAUGGUGCACUUCUU | |
| miR-500 | >hsa-miR-500 MIMAT0004773 Homo sapiens miR-500 | 300 |
| | UAAUCCUUGCUACCUGGGUGAGA | |
| | >hsa-miR-500* MIMAT0002871 Homo sapiens miR-500* | 301 |
| | AUGCACCUGGGCAAGGAUUCUG | |
| miR-501-5p | >hsa-miR-501-5p MIMAT0002872 Homo sapiens miR-501-5p | 302 |
| | AAUCCUUUGUCCCUGGGUGAGA | |
| miR-502-3p | >hsa-miR-502-3p MIMAT0004775 Homo sapiens miR-502-3p | 303 |
| | AAUGCACCUGGGCAAGGAUUCA | |
| miR-503 | >hsa-miR-503 MIMAT0002874 Homo sapiens miR-503 | 304 |
| | UAGCAGCGGGAACAGUUCUGCAG | |
| miR-539 | >hsa-miR-539 MIMAT0003163 Homo sapiens miR-539 | 305 |
| | GGAGAAAUUAUCCUUGGUGUGU | |
| miR-542-5p | >hsa-miR-542-5p MIMAT0003340 Homo sapiens miR-542-5p | 306 |
| | UCGGGGAUCAUCAUGUCACGAGA | |
| miR-543 | >hsa-miR-543 MIMAT0004954 Homo sapiens miR-543 | 307 |
| | AAACAUUCGCGGUGCACUUCUU | |
| miR-549 | >hsa-miR-549 MIMAT0003333 Homo sapiens miR-549 | 308 |
| | UGACAACUAUGGAUGAGCUCU | |
| miR-590-5p | >hsa-miR-590-5p MIMAT0003258 Homo sapiens miR-590-5p | 309 |
| | GAGCUUAUUCAUAAAAGUGCAG | |
| miR-625 | >hsa-miR-625 MIMAT0003294 Homo sapiens miR-625 | 310 |
| | AGGGGGAAAGUUCUAUAGUCC | |
| miR-628-5p | >hsa-miR-628-5p MIMAT0004809 Homo sapiens miR-628-5p | 311 |
| | AUGCUGACAUAUUUACUAGAGG | |
| miR-655 | >hsa-miR-655 MIMAT0003331 Homo sapiens miR-655 | 312 |
| | AUAAUACAUGGUUAACCUCUUU | |
| miR-660 | >hsa-miR-660 MIMAT0003338 Homo sapiens miR-660 | 313 |
| | UACCCAUUGCAUAUCGGAGUUG | |
| miR-668 | >hsa-miR-668 MIMAT0003881 Homo sapiens miR-668 | 314 |
| | UGUCACUCGGCUCGGCCCACUAC | |
| miR-758 | >hsa-miR-758 MIMAT0003879 Homo sapiens miR-758 | 315 |
| | UUUGUGACCUGGUCCACUAACC | |
| miR-769 | >hsa-miR-769-5p MIMAT0003886 Homo sapiens miR-769-5p | 316 |
| | UGAGACCUCUGGGUUCUGAGCU | |
| | >hsa-miR-769-3p MIMAT0003887 Homo sapiens miR-769-3p | 317 |
| | CUGGGAUCUCCGGGGUCUUGGUU | |
| miR-886-3p | >hsa-miR-886-3p MIMAT0004906 Homo sapiens miR-886-3p | 318 |
| | CGCGGGUGCUUACUGACCCUU | |
| miR-886-5p | >hsa-miR-886-5p MIMAT0004905 Homo sapiens miR-886-5p | 319 |
| | CGGGUCGGAGUUAGCUCAAGCGG | |

It is contemplated that one or more microRNAs identified according to the present invention (e.g., SEQ ID NOs: 1 to 610 (or SEQ ID NOs: 1 to 225) may be used to induce or stimulate tissue or cell repair, growth, remodeling, reconstruction, differentiation and/or transdifferentitation, and/or to treat associated diseases, disorders or conditions. In some embodiments, functional variants of microRNAs described herein may be used. For example, suitable microRNAs may include microRNAs having a sequence at least 70% (e.g., 75%, 80%, 85%, 90%, 95%, 96%, 97%, 98%, 99%) identical to any one of microRNAs identified in Table 1 and Tables 6-10. In some embodiments, suitable microRNAs are functional variants of microRNAs that are specifically present in pathfinder cells. Accordingly, in some embodiments, suitable microRNAs may include microRNAs having a sequence at least 70% (e.g., 75%, 80%, 85%, 90%, 95%, 96%, 97%, 98%, 99%) identical to any one of SEQ ID NOs: 1 to 610 or SEQ ID NOs: 1 to 225.

“Percent (%) nucleic acid sequence identity” with respect to microRNA sequences identified herein is defined as the percentage of nucleotides in a candidate sequence that are identical with the nucleotides in a reference sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, ALIGN or Megalign (DNASTAR) software. Those skilled in the art can determine appropriate parameters for measuring alignment, including any algorithms needed to achieve maximal alignment over the full length of the sequences being compared. Preferably, the WU-BLAST-2 software is used to determine amino acid sequence identity (Altschul et al., Methods in Enzymology, 266, 460-480 (1996); http://blast.wustl/edu/blast/README.html). WU-BLAST-2 uses several search parameters, most of which are set to the default values. The adjustable parameters are set with the following values: overlap span=1, overlap fraction=0.125, world threshold (T)=11. HSP score (S) and HSP S2 parameters are dynamic values and are established by the program itself, depending upon the composition of the particular sequence, however, the minimum values may be adjusted and are set as indicated above.

Suitable microRNAs may be comprised entirely of natural RNA nucleotides, or may instead include one or more nucleotide analogs and/or modifications. The microRNA structure may be stabilized, for example by including nucleotide analogs at one or more free strand ends in order to reduce digestion, e.g., by exonucleases. Suitable microRNAs may contain modified ribonucleotides, that is, ribonucleotides that contain a modification in the chemical structure of an unmodified nucleotide base, sugar and/or phosphate (or phosphodiester linkage). As is known in the art, an “unmodified ribonucleotide” has one of the bases adenine, cytosine, guanine, and uracil joined to the l′ carbon of beta-D-ribo-furanose. Modified microRNA molecules may also contain modified backbones or non-natural internucleoside linkages, e.g., modified phosphorous-containing backbones and non-phosphorous backbones such as morpholino backbones; siloxane, sulfide, sulfoxide, sulfone, sulfonate, sulfonamide, and sulfamate backbones; formacetyl and thioformacetyl backbones; alkene-containing backbones; methyleneimino and methylenehydrazino backbones; amide backbones, and the like.

### III. Extracellular Secretomes

As used herein, extracellular secretomes of pathfinder cells refer to any isolates derived from pathfinder cells according to the invention, including, but not limited to, any microvesicles including exosomes isolated from Pathfinder cells. Various methods of isolating or enriching microvesicles known in the art may be used to practice the present invention. As used herein, the terms “isolation” or “isolating” in conjunction with microvesicles are interchangeably used with the terms “enrichment” or “enriching,” and refer to one or more process steps that result in an increase of the fraction of microvesicles in a sample as compared to the fraction of microvesicles in the obtained biological sample. Thus, microvesicles may be purified to homogeneity, purified to at least 90% (with respect to non-microvesicle particulate matter), at least 80%, at least 70%, at least 60%, at least 50%, at least 40%, at least 30%, or at least 20% (or even less). For example, physical properties of microvesicles—may be employed to separate them from a medium or other source material. For example, microvesicles may be separated on the basis of electrical charge (e.g., electrophoretic separation), size (e.g., filtration, molecular sieving, etc), density (e.g., regular or gradient centrifugation), Svedberg constant (e.g., sedimentation with or without external force, etc).

In some embodiments, microvesicles are isolated or purified by centrifugation (e.g., ultracentrifugation). It will be appreciated that various centrifugation conditions (e.g., speed, centrifugal force, centrifugation time, etc.) may be used in order to obtain a desired fraction of isolated or purified microvesicles. For example, in some embodiments, a sample may be centrifuged at a fairly low centrifugal force (e.g., approximately 16,000×g) sufficient to pellet larger microvesicles (e.g., approximately 1000 nm or more). In some embodiments, a sample (e.g., the resulting supernatant from the initial low speed spin) may be centrifuged at a higher centrifugal force (e.g., approximately 120,000×g) sufficient to pellet microvesicles of a smaller size (e.g., less then 1000 nm). In some embodiments, a microvesicle preparation prepared using this method may contain substantially small particles, for example, particles with a size ranging from about 10 nm to 1000 nm (e.g., about 50-1000 nm, 75-1000 nm, 100-1000 nm, 10-750 nm, 50-750 nm, 100-750 nm, 100-500 nm). In some embodiments, such small particles are also referred to as exosomes, exosome-like vesicles, and/or membrane particles. In some embodiments, such fraction is referred to as exosome fraction.

In some embodiments, microvesicles are isolated or purified by precipitation. It will be appreciated that various precipitation conditions may be used in order to obtain a desired fraction of isolated or purified microvesicles. For example, various kits are available for exosome precipitation, such as ExoQuick™ and Exo-Quick-TC™ (available from System Biosciences, Mountain View, Calif.) and may be used in accordance with the present invention.

Alternatively, or additionally, isolation may be based on one or more biological properties, and may employ surface markers (e.g., for precipitation, reversible binding to solid phase, FACS separation, specific ligand binding, non-specific ligand binding such as annexin V, etc.). In yet further contemplated methods, the microvesicles may also be fused using chemical and/or physical methods, including PEG-induced fusion and/or ultrasonic fusion.

In some embodiments, microvesicles are obtained from conditioned media from cultures of microvesicle-producing cells.

Synthetic Microvesicles

In some embodiments, microvesicles suitable for the present invention may be synthetically produced. Synthetic microvesicles typically include one or more membrane components obtained from a donor cell. In some embodiments, synthetic microvesicles include at least one microRNA described herein. For example, synthetic microvesicles may be prepared by disintegration of a donor cell (e.g., via detergent, sonication, shear forces, etc.) and use of the crude preparation or an at least partially enriched membrane fraction to reconstitute one or more microvesicles. In some embodiments, exogenous microRNAs may be added to microvesicles.

Further detailed description of microvesicles and uses thereof are provide in PCT application entitled “THERAPEUTIC USES OF MICROVESICLES AND RELATED MICRORNAS” filed on even date and claiming priority to U.S. Provisional Patent Application Ser. No. 61/373,715 filed on Aug. 13, 2010, the entirety of which is incorporated herein by reference.

### III. Therapeutic Applications

In some embodiments, the present invention provides methods of using pathfinder cells, cells differentiated from pathfinder cells, extracellular secretomes (e.g., microvesicles) and/or microRNAs for inducing or stimulating tissue or cell repair, growth, remodeling, reconstruction, differentiation and/or transdifferentitation, or treating associated diseases, disorders or conditions. While not wishing to be bound by a particular theory or hypothesis, it is contemplated that pathfinder cells may induce changes within target tissue or cells to convert them into active repair mode by providing microRNAs and/or other components (e.g., membrane associated polypeptide, transcription factors, etc.) that will regulate expression of genes relating to, e.g., increased cell mobility, tissue remodeling and reprogramming, growth, angiogenesis, cell adhesion and cell signaling, etc. It is further contemplated that pathfinder cells will typically not be part of the new tissue or cells. Thus, according to the present invention, pathfinder cells or microRNAs from different tissues, cell types or organisms may be used. In some embodiments, pathfinder cells or microRNAs may be used without inducing an immune reaction. In some embodiments, pathfinder cells or microRNAs may be used without an immunosuppressant.

Thus, suitable pathfinder cells or microRNAs can be derived from autologous cells (i.e., cells from the same individual as the patient) or non-autologous cells (i.e., cells from a different individual as the patient) or both. In some embodiments, pathfinder cells are derived from tissue that is the same as the diseased tissue. For example, in methods of treating a kidney disease, pathfinder cells may be taken from healthy kidney cells from the same or different individual being treated. In some embodiments, pathfinder cells are derived from tissue that is different than the diseased tissue.

In some embodiments, methods of treatment comprise one or more steps that are performed in vitro or ex vivo to induce cells (“recipient cells”) to differentiate or transdifferentiate into a desirable cell type. Such recipient cells can then be transferred into a patient.

Without wishing to be bound by any particular theory, the inventors have proposed that pathfinder cells may act on other cells at least partially through microvesicles. (See U.S. provisional application Ser. No. 61/373,715, filed on Aug. 13, 2010, the entire contents of which are incorporated herein by reference). According to this theory, microvesicles produced by pathfinder cells contain factors (e.g., microRNAs) that confer desirable properties on recipient cells (i.e., cells that receive microvesicles, their contents, and/or or other factors secreted or produced by pathfinder cells).

In some embodiments, provided methods comprise co-culturing pathfinder cells and recipient cells ex vivo and then transferring recipient cells into an patient. In some embodiments, recipient cells are transferred back into the same individual from whom recipient cells were obtained. For example, pathfinder cells can be co-cultured with bone marrow cells obtained from a patient for a period of time ex vivo to allow transfer of stimulatory factors (e.g., microvesicles, their contents, and/or other factors secreted or produced by pathfinder cells), then bone marrow cells may be transferred back into the individual.

In some embodiments, recipient cells are tested for expression of specific biomarkers such as certain proteins and/or microRNAs after co-culturing with pathfinder cells before transfer into a patient.

In certain embodiments, methods of treatment comprise a step of administering to a patient in need of treatment a therapeutically effective amount of one or more microRNAs as described herein. miRNAs may be used in the absence or presence of cells.

In certain embodiments, methods and compositions of the present invention are used to stimulate repair of tissues and/or cells that have are damaged acutely (e.g., by trauma, and/or due to an acute disease, disorder, or condition). Results described herein provide the first evidence to the inventor's knowledge that pluripotent adult cells can successfully stimulate repair of acute damage to tissues. It is contemplated that pathfinder cell-associated microRNAs disclosed herein would similarly be useful in stimulating repair of acute damage.

In some embodiments, methods and compositions of the present invention are used to stimulate repair of damaged tissue in an acute condition resulting from ischemia. By way of non-limiting example, methods and compositions of the present invention may be used to treat stroke, subarachnoid hemorrhage, brain hemorrhage, hemorrhagic stroke, brain trauma, head injury, head trauma, seizure, a headache disorder (e.g., migraine or cluster headache), cardiovascular disease (e.g., myocardial infarction, heart disease, coronary artery disease, congestive heart failure, cardiac valvular disease, cardiac arrhythmia, and cardiac arrest), tissue organ engraftment rejection, sequelae of ischemic reperfusion injury, retinal ischemia (e.g., diabetic retinopathy, central retinal artery or vein occlusion, stenosis of the carotid artery, and sickle cell retinopathy), retinal detachment, retinal tearing, gastrointestinal ischemia (e.g., ischemic bowel, ischemic colitis, and mesenteric ischemia), renal (kidney) ischemia, peripheral ischemia (e.g., acute peripheral vascular ischemia, atherosclerosis, peripheral arterial occlusive disease, thromboembolic disease, and thromboangiitis obliterans (Buerger's disease). In some embodiments, methods and compositions of the invention are used to stimulate repair in individuals who suffer from stroke, renal ischemia, or myocardial infarct.

In some embodiments, methods and compositions of the present invention are used to treat reperfusion injury. Reperfusion injury refers to damage to a tissue caused when blood supply returns to the tissue after a period of ischemia. Reperfusion injury is thought to arise at least in part from the inflammatory response of damaged tissues. Blood cells in newly returning blood may release a host of inflammatory factors such as interleukins and free radicals in response to the tissue damage. Oxidative stress resulting from reintroduced oxygen from returning blood flow may also contribute to reperfusion injury.

In some embodiments, methods and compositions of the present invention are used to treat or alleviate acute organ failure, such as, for example, kidney failure or liver failure.

In some embodiments, methods and compositions of the present invention are used to treat or alleviate acute complications of diseases. For example, in Type I diabetes (also known as “juvenile diabetes”), acute complications may arise suddenly from alterations in blood glucose levels; such complications include, but are not limited to, ketoacidosis (a pathological metabolic state marked by extreme and uncontrolled ketosis), hyperglycemic hyperosmolar non-ketotic syndrom (HHNS) (usually associated with insulin deficiency and dehydration), and hypoglycemia (usually associated with too much insulin and/or too little glucose).

Inflammation

In some embodiments, methods and compositions of the present invention are used to treat or ameliorate inflammation. As used herein, the term “inflammation” includes inflammatory conditions occurring in many disorders which include, but are not limited to: Systemic Inflammatory Response (SIRS); Alzheimer's Disease (and associated conditions and symptoms including: chronic neuroinflammation, glial activation; increased microglia; neuritic plaque formation; and response to therapy); Amyotropic Lateral Sclerosis (ALS), arthritis (and associated conditions and symptoms including, but not limited to: acute joint inflammation, antigen-induced arthritis, arthritis associated with chronic lymphocytic thyroiditis, collagen-induced arthritis, juvenile arthritis; rheumatoid arthritis, osteoarthritis, prognosis and streptococcus-induced arthritis, spondyloarthopathies, gouty arthritis), asthma (and associated conditions and symptoms, including: bronchial asthma; chronic obstructive airway disease; chronic obstructive pulmonary disease, juvenile asthma and occupational asthma); cardiovascular diseases (and associated conditions and symptoms, including atherosclerosis; autoimmune myocarditis, chronic cardiac hypoxia, congestive heart failure, coronary artery disease, cardiomyopathy and cardiac cell dysfunction, including: aortic smooth muscle cell activation; cardiac cell apoptosis; and immunomodulation of cardiac cell function; diabetes and associated conditions and symptoms, including autoimmune diabetes, insulin-dependent (Type 1) diabetes, diabetic periodontitis, diabetic retinopathy, and diabetic nephropathy); gastrointestinal inflammations (and related conditions and symptoms, including celiac disease, associated osteopenia, chronic colitis, Crohn's disease, inflammatory bowel disease and ulcerative colitis); gastric ulcers; hepatic inflammations such as viral and other types of hepatitis, cholesterol gallstones and hepatic fibrosis, HIV infection (and associated conditions and symptoms, including degenerative responses, neurodegenerative responses, and HIV associated Hodgkin's Disease), Kawasaki's Syndrome (and associated diseases and conditions, including mucocutaneous lymph node syndrome, cervical lymphadenopathy, coronary artery lesions, edema, fever, increased leukocytes, mild anemia, skin peeling, rash, conjunctiva redness, thrombocytosis; multiple sclerosis, nephropathies (and associated diseases and conditions, including diabetic nephropathy, endstage renal disease, acute and chronic glomerulonephritis, acute and chronic interstitial nephritis, lupus nephritis, Goodpasture's syndrome, hemodialysis survival and renal ischemic reperfusion injury), neurodegenerative diseases (and associated diseases and conditions, including acute neurodegeneration, induction of IL-1 in aging and neurodegenerative disease, IL-1 induced plasticity of hypothalamic neurons and chronic stress hyperresponsiveness), ophtlialmopathies (and associated diseases and conditions, including diabetic retinopathy, Graves' opthalmopathy, and uveitis, osteoporosis (and associated diseases and conditions, including alveolar, femoral, radial, vertebral or wrist bone loss or fracture incidence, postmenopausal bone loss, mass, fracture incidence or rate of bone loss), otitis media (adult or pediatric), pancreatitis or pancreatic acinitis, periodontal disease (and associated diseases and conditions, including adult, early onset and diabetic); pulmonary diseases, including chronic lung disease, chronic sinusitis, hyaline membrane disease, hypoxia and pulmonary disease in SIDS; restenosis of coronary or other vascular grafts; rheumatism including rheumatoid arthritis, rheumatic Aschoff bodies, rheumatic diseases and rheumatic myocarditis; thyroiditis including chronic lymphocytic thyroiditis; urinary tract infections including chronic prostatitis, chronic pelvic pain syndrome and urolithiasis. Immunological disorders, including autoimmune diseases, such as alopecia aerata, autoimmune myocarditis, Graves' disease, Graves opthalmopathy, lichen sclerosis, multiple sclerosis, psoriasis, systemic lupus erythematosus, systemic sclerosis, thyroid diseases (e.g. goiter and struma lymphomatosa (Hashimoto's thyroiditis, lymphadenoid goiter), sleep disorders and chronic fatigue syndrome and obesity (non-diabetic or associated with diabetes). Resistance to infectious diseases, such as Leishmaniasis, Leprosy, Lyme Disease, Lyme Carditis, malaria, cerebral malaria, meningitis, tubulointerstitial nephritis associated with malaria), which are caused by bacteria, viruses (e.g. cytomegalovirus, encephalitis, Epstein-Barr Virus, Human Immunodeficiency Virus, Influenza Virus) or protozoans (e.g., Plasmodium falciparum, trypanosomes). Response to trauma, including cerebral trauma (including strokes and ischemias, encephalitis, encephalopathies, epilepsy, perinatal brain injury, prolonged febrile seizures, SIDS and subarachnoid hemorrhage), low birth weight (e.g. cerebral palsy), lung injury (acute hemorrhagic lung injury, Goodpasture's syndrome, acute ischemic reperfusion), myocardial dysfunction, caused by occupational and environmental pollutants (e.g. susceptibility to toxic oil syndrome silicosis), radiation trauma, and efficiency of wound healing responses (e.g. burn or thermal wounds, chronic wounds, surgical wounds and spinal cord injuries). Hormonal regulation including fertility/fecundity, likelihood of a pregnancy, incidence of preterm labor, prenatal and neonatal complications including preterm low birth weight, cerebral palsy, septicemia, hypothyroidism, oxygen dependence, cranial abnormality, early onset menopause. A subject's response to transplant (rejection or acceptance), acute phase response (e.g. febrile response), general inflammatory response, acute respiratory distress response, acute systemic inflammatory response, wound healing, adhesion, immunoinflammatory response, neuroendocrine response, fever development and resistance, acute-phase response, stress response, disease susceptibility, repetitive motion stress, tennis elbow, and pain management and response.

In particular embodiments, methods and compositions of the present invention can be used to treat or ameliorate inflammation associated with an immunodeficiency disease, disorder, or condition. Non-limiting examples of diseases, disorders, and conditions that may be characterized by immunodeficiency include hypgammaglobulinemia, agammaglobulinemia, ataxia telengiectasia, severe combined immunodeficiency disease (SCID), acquired immunodeficiency syndrome (AIDS) such as that caused by infection by human immunodeficiency virus (HIV), Chediak-Higashi syndrome, combined immunodeficiency disease, complement deficiencies, diGeorge syndrome, Job syndrome, leukocyte adhesion defects, panhypogammaglobulinemia (e.g., Bruton disease, congential agammaglobulinemia, selective deficiency of IgA, Wiscott-Aldrich syndrome. In some embodiments, pathfinder cells and/or cells differentiated from pathfinder cells treat or ameliorate immunodeficiency by stimulating reconstitution of one or more blood cell types, i.e., cells of the immune system. It is contemplated that pathfinder cell-associated microRNAs disclosed herein would similarly be useful in treating or ameliorating immunodeficiency.

In certain embodiments, methods and compositions of the present invention are used to treat or ammeliorate an autoimmune diesase, disorder or condition. In general, autoimmunity is the failure of an organism to recognize its own constituent parts as “self,” which results in an immune response against the organism's own tissues and cells. Exemplary autoimmune diseases and/or suspected autoimmune diseases include, but are not limited to, Acute disseminated encephalomyelitis (ADEM), Addison's disease, Alopecia universalis, Ankylosing spondylitisis, Antiphospholipid antibody syndrome (APS), Aplastic anemia, Autoimmune hemolytic anemia, Autoimmune hepatitis, Autoimmune inner ear disease (AIED), Autoimmune lymphoproliferative syndrome (ALPS), Autoimmune oophoritis, Balo disease, Behcet's disease, Bullous pemphigoid, Cardiomyopathy, Chagas' disease, Chronic fatigue immune dysfunction syndrome (CFIDS), Chronic inflammatory demyelinating polyneuropathy, Crohn's disease, Cicatrical pemphigoid, Coeliac sprue-dermatitis herpetiformis, Cold agglutinin disease, CREST syndrome, Degos disease, Diabetes mellitus, Discoid lupus, Dysautonomia, Endometriosis, Essential mixed cryoglobulinemia, Fibromyalgia-fibromyositis, Goodpasture's syndrome, Grave's disease, Guillain-Barré syndrome (GBS), Hashimoto's thyroiditis, Hidradenitis suppurativa, Idiopathic and/or acute thrombocytopenic purpura, Idiopathic pulmonary fibrosis, IgA neuropathy, Interstitial cytisis, Juvenile arthritis, Kawasaki's disease, Lichen planus, Lupus erythematosus, Lyme disease, Ménière disease, Mixed connective tissue disease (MCTD), Multiple sclerosis, Myasthenia gravis, Neuromyotonia, Opsoclonus myoclonus syndrome (OMS), Optic neuritis, Ord's thyroiditis, Osteoarthritis, Pemphigus vulgaris, Pernicious anemia, Polyarthritis, Polychondritis, Polymyositis and dermatomyositis, Primary biliary cirrhosis, Psoriasis, Polyarteritis nodosa, Polyglandular syndromes, Polymyalgia rheumatica, Primary agammaglobulinemia, Raynaud phenomenon, Reiter's syndrome, Rheumatic fever, Sarcoidosis, Schizophrenia, Scleroderma, Sjogren's syndrome, Stiff person syndrome, Takayasu's arteritis, Temporal arteritis (also known as “giant cell arteritis”), Ulcerative colitis, Uveitis, Vasculitis, Vitiligo, Vulvodynia (“vulvar vestibulitis”), and Wegener's granulomatosis.

Transplantation Stress

In certain embodiments, methods and compositions of the present invention are used to alleviate transplantation stress. It is contemplated that tissue/organ transplantation may cause acute tissue damage and pathfinder cells, extracellular secretomes (e.g., microvesicles) and associated microRNAs disclosed herein may be administered into an organ/tissue transplant recipient to stimulate tissue repair, regeneration, reconstitution, remodeling, and/or inducing immune tolerance, thereby alleviating transplantation stress. It is contemplated that the present invention may be used to facilitate any organ transplantation including, but not limited to, heart, kidney, liver, lung, pancreas, intestine, thymus, and skin transplantation.

In certain embodiments, methods and compositions of the present invention are used to treat or ameliorate a disease, disorder, or condition associated with graft rejection. In general, graft rejection may result from functional immune cells in a recipient recognizing a donor organ or tissue as a foreign entity and mounting of an immunologic attack on the donor organ or tissue. In some cases, graft rejection arises in an acute phase following transplantation of donor organs or tissues to a recipient. In some cases, graft rejection arises in a chronic phase following transplantation of donor organs or tissues to a recipient. It is to be understood that the present invention encompasses methods and compositions for treatment of acute and/or chronic graft rejection.

In certain embodiments, methods and compositions of the present invention are used to treat or ameliorate a graft versus host disease, disorder, or condition. In general, Graft versus Host disease (GVHD) may result from functional immune cells in a transplanted tissue or organ from a donor recognizing the recipient as a foreign entity and mounting an immunologic attack on the recipient's cells and/or tissues. In some cases, GVHD arises in an acute phase following transplantation of donor organs or tissues to a recipient. In some cases, GVHD arises in a chronic phase following transplantation of donor organs or tissues to a recipient. It is to be understood that the present invention encompasses methods and compositions for treatment of acute and/or chronic GVHD.

Immune Tolerance

It is contemplated that pathfinder cells or their extracellular secretomes (e.g., microvesicles) induce immune tolerance and thus are particularly useful in treating inflammation and suppressing, inhibiting or reducing transplantation associated stress. Without wishing to be bound by particular theory, it is contemplated that the pathfinder cells or their extracellular secretomes (e.g., microvesicles) induce immune tolerance by inducing increased IL-2 response, resulting in expansion of regulatory T cells (e.g., increased level and/or activity of T regulatory cells), decreased level and/or activity of cytotoxic T cells and/or helper T cells, and/or suppression of T cell or non T cell lymphocyte responses. In some embodiments, pathfinder cells or their extracellular secretomes (e.g., microvesicles) suppress pro-inflammatory and/or anti-angiogenic cytokine or chemokine response. Pro-inflammatory and/or anti-angiogenic cytokines or chemokines are well known in the art. Exemplary pro-inflammatory and/or anti-angiogenic cytokines or chemokines include, but are not limited to, IL-4, IL-5, IL-6, IL-10, IL-12, IL-13, IL-17, GMCSF, TGF-β, TNF-α, IFN-γ, MCAF, and MIP1. As shown in the Examples section, cells or their extracellular secretomes (e.g., microvesicles) according to the present invention have unexpectedly distinct cytokine or chemokine profiles as compared to Mesenchymal stem cells (MSCs). For example, pathfinder cells according to the present invention reduce the expression or activity of IL-4, IL-6 and/or IL-10, while MSCs are known to increase the expression or activity of IL-4, IL-6 and IL-10. Other distinctions are shown in Table 7. In some embodiments, cells or their extracellular secretomes (e.g., microvesicles) increase anti-inflammatory and/or pro-angiogenic cytokine or chemokine response. Anti-inflammatory and/or pro-angiogenic cytokines or chemokines are known in the art. Exemplary anti-inflammatory and/or pro-angiogenic cytokines or chemokines include, but are not limited to, IL-1β, GSCF, and IL-8.

Accordingly, administration of pathfinder cells or their extracellular secretomes (e.g., microvesicles) according to the present invention does not result in severe adverse effects in the subject. As used herein, severe adverse effects include, but are not limited to, substantial immune response, toxicity, or death. As used herein, the term “substantial immune response” refers to severe or serious immune responses, such as adaptive T-cell immune responses.

Thus, in many embodiments, inventive methods according to the present invention do not involve concurrent immunosuppressant therapy (i.e., any immunosuppressant therapy used as pre-treatment/pre-conditioning or in parallel to the method). In some embodiments, inventive methods according to the present invention do not involve an immune tolerance induction in the subject being treated. In some embodiments, inventive methods according to the present invention do not involve a pre-treatment or preconditioning of the subject using T-cell immunosuppressive agent.

In some embodiments, however, administration of pathfinder cells or their extracellular secretomes (e.g., microvesicles) according to the present invention can mount an immune response against these agents. Thus, in some embodiments, it may be useful to render the subject receiving the cells or their extracellular secretomes (e.g., microvesicles) tolerant to the therapy. Immune tolerance may be induced using various methods known in the art. Any immunosuppressant agent known to the skilled artisan may be employed together with a combination therapy of the invention. Such immunosuppressant agents include but are not limited to cyclosporine, FK506, rapamycin, CTLA4-Ig, and anti-TNF agents such as etanercept (see e.g. Moder, 2000, Ann. Allergy Asthma Immunol. 84, 280-284; Nevins, 2000, Curr. Opin. Pediatr. 12, 146-150; Kurlberg et al., 2000, Scand. J. Immunol. 51, 224-230; Ideguchi et al., 2000, Neuroscience 95, 217-226; Potter et al., 1999, Ann. N.Y. Acad. Sci. 875, 159-174; Slavik et al., 1999, Immunol. Res. 19, 1-24; Gaziev et al., 1999, Bone Marrow Transplant. 25, 689-696; Henry, 1999, Clin. Transplant. 13, 209-220; Gummert et al., 1999, J. Am. Soc. Nephrol. 10, 1366-1380; Qi et al., 2000, Transplantation 69, 1275-1283). The anti-IL2 receptor (.alpha.-subunit) antibody daclizumab (e.g. Zenapax™), which has been demonstrated effective in transplant patients, can also be used as an immunosuppressant agent (see e.g. Wiseman et al., 1999, Drugs 58, 1029-1042; Beniaminovitz et al., 2000, N. Engl J. Med. 342, 613-619; Ponticelli et al., 1999, Drugs R. D. 1, 55-60; Berard et al., 1999, Pharmacotherapy 19, 1127-1137; Eckhoff et al., 2000, Transplantation 69, 1867-1872; Ekberg et al., 2000, Transpl. Int. 13, 151-159). Additional immunosuppressant agents include but are not limited to anti-CD2 (Branco et al., 1999, Transplantation 68, 1588-1596; Przepiorka et al., 1998, Blood 92, 4066-4071), anti-CD4 (Marinova-Mutafchieva et al., 2000, Arthritis Rheum. 43, 638-644; Fishwild et al., 1999, Clin. Immunol. 92, 138-152), and anti-CD40 ligand (Hong et al., 2000, Semin. Nephrol. 20, 108-125; Chirmule et al., 2000, J. Virol. 74, 3345-3352; Ito et al., 2000, J. Immunol. 164, 1230-1235).

In addition, methods and compositions (e.g., pathfinder cells, cells differentiated from pathfinder cells, microvesicles and/or microRNAs) according to the present invention may be used to treat diseases, disorders, or conditions in various tissues including, but not limited to, central nervous system (CNS), peripheral nervous system, cardiovascular system, respiratory system, gastrointestinal tract and associated glands, integumentary system, musculoskeletal system, and other systems of the body. In some embodiments, methods and compositions according to the present invention may be used to treat age-related degeneration as well as progerias. In some embodiments, methods and compositions according to the present invention may be used to treat inflammation. In some embodiments, cells and/or microRNAs according to the present invention may be suitable for cosmetic uses or for treating a condition or disorder associated with a cosmetic surgical procedure.

Central Nervous System (CNS)

Examples of CNS-related diseases, disorders or conditions that may be treated by the methods and compositions of the present invention include motor neuron disease, multiple sclerosis, degenerative diseases of the CNS, dementive illnesses such as Alzheimer's disease, age related dysfunction of the CNS, Parkinson's disease, cerebrovascular accidents, epilepsy, temporary ischaemic accidents, disorders of mood, psychotic illnesses, specific lobe dysfunction, pressure related injury, cognitive dysfunction or impairments, deafness, blindness anosmia, diseases of the special senses, motor deficits, sensory deficits, head injury and trauma to the CNS. Methods and products of the present invention may also be used to enhance brain function or ameliorate deficiencies at a functional level or to facilitate post surgical repair of the CNS.

Cardiovascular System

Examples of diseases, disorders or conditions of the cardiovascular system that may be treated by the methods and compositions of the present invention include arrhythmias, myocardial infarction and other heart attacks, pericarditis, congestive heart diseases, valve-related pathologies, myocardial, endocardial and pericardial dysfunctions or degeneration, age-related cardiovascular disorders, dysfunctions, degeneration or diseases, sclerosis and thickening of valve flaps, fibrosis of cardiac muscle, decline in cardiac reserve, congenital defects of the heart or circulatory system, developmental defects of the heart or circulatory system, repair of hypoxic or necrotic damage, blood vessel damage and cardiovascular diseases or dysfunction (e.g., angina, dissected aorta, thrombotic damage, aneurysm, atherosclerosis, emboli damage and other problems associated with blood flow, pressure or impediment). Methods and compositions of the present invention may also be used to enhance cardiovascular function or health and to revascularise tissues. Moreover, methods and compositions of the present invention may be used to repair, modify, enhance or regenerate traumatic damage to the heart or blood vessels and as a technique to enhance the transplantation/implantation of a whole organ or its parts. Examples of this latter embodiment include heart transplantation, valve replacement surgeries, implantation of prosthetic devices and the development of novel surgical techniques.

Respiratory System

Examples of diseases, disorders or conditions of the respiratory system that may be treated by the methods and compositions of the present invention include damage, pathology, ageing and trauma of the nose and paranasal sinuses, nasopharynx, oropharynx, laryngopharynx, larynx, vocal ligaments, vocal cords, vestibular folds, glottis, epiglottis, trachea, mucocilliary mucosa, trachealis muscle, primary bronchi, lobar bronchi, segmental bronchi, terminal bronchioles, respiratory zone structures and plural membranes. Examples of such damage include obstructive pulmonary diseases, restrictive disorders, emphysema, chronic bronchitis, pulmonary infections, asthma, tuberculosis, genetic disorders (e.g., cystic fibrosis), gas exchange problems, burns, barotraumas and disorders affecting blood supply to the respiratory system. Methods and medicaments of the present invention may also be used to repair, modify, enhance or regenerate the respiratory system following damage. Moreover, methods and compositions of the present invention may be used as a technique to enhance the transplantation/implantation of whole respiratory structures or organs or their parts.

Gastrointestinal Tract and Associated Glands

Examples of diseases, disorders or conditions of the gastrointestinal tract and associated glands that may be treated by the methods and medicaments of the present invention include disorders, damage and age related changes of both the gastrointestinal tract and the large accessory glands (liver and pancreas), salivary glands, mouth, teeth, oesophagus, stomach, duodenum, jejunum, ileum, ascending colon, transverse colon, descending colon, sigmoid colon, rectum and anal canal and enteric nervous system of the canal. In specific embodiments, these disorders, damage and age related changes include dental caries, periodontal disease, deglutition problems, ulcers, enzymatic disturbances/deficiencies, motility problems, paralysis, dysfunction of absorption or absorptive surfaces, diverticulosis, inflammatory bowel problems, hepatitis, cirrhosis and portal hypertension. Methods and medicaments of the present invention may also be used to repair, modify, enhance or regenerate the gastrointestinal tract following damage, or be used as a technique to enhance any of these processes following surgery, such as resection of the stomach, ileostomy and reconstructive surgery (eg ileoanal juncture). Examples of this latter embodiment include reconstructive surgery involving specific anatomical structures of the mouth, such as labia, vestibule, oral cavity proper, red margin, labial frenulum, hard palate palatine bones, soft palate, uvula, tongue, intrinsic muscles of the tongue and extrinsic muscles of the tongue.

Integumentary System

Examples of diseases, disorders or conditions of the integumentary system that may be treated by the methods and medicaments of the present invention include disorders, damage and age related changes of the skin and integumentary system, such as age related decline in thickness or function, disorders of sweat gland and sebaceous glands, piloerectile dysfunction, follicular problems, hair loss, epidermal disease, diseases of the dermis or hypodermis, burns, ulcers, sores and infections. Methods and products of the present invention may also be used to enhance, regenerate or repair skin structures or functions, for example in plastic reconstruction, cosmetic repair, tattoo removal, wound healing, modulation of wrinkles and in the treatment of striae, seborrhoea, rosacea, port wine stains, skin colour and the improvement of blood supply to the skin. Moreover, methods and products of the present invention may be used to enhance skin grafts, surgical reconstruction, cosmetic surgical procedures, wound healing and cosmetic appearance.

Musculoskeletal System

Examples of diseases, disorders or conditions of the musculoskeletal system that may be treated by the methods and products of the present invention include disease, damage and age related changes of the musculoskeletal system. In some embodiment, these may be in components of the axial skeleton, including the skull, cranium, face, skull associated bones, auditory ossicles, hyoid bone, sternum, ribs, vertebrae, sacrum and coccyx. In other embodiments they may be in components of the appendicular skeleton, including the clavicle, scapula, humerus, radius, ulna, carpal bones, metacarpal bones, phalanges (proximal, middle, distal), pelvic girdle, femur, patella, tibia, fibula, tarsal bones and metatarsal bones. Methods and compositions of the present invention may also be used to correct problems associated with ossification and osteogenesis, such as intramembranous ossification, endochondral ossification, bone remodelling and repair, osteoporosis, osteomalacia, rickets, pagets disease, rheumatism and arthritis. Moreover, methods and products of the present invention may be used to treat disease, damage and age related changes of the skeletal muscle, elastic cartilages, fibrocartilages, long bones, short bones, flat bones and irregular bones.

Other Systems of the Body

Diseases, disorders or conditions of other systems of the body may be treated by the methods and products of the present invention. For example, the present invention may be used to enhance function or treat disease, damage and age related changes in other systems of the body, including special senses, endocrine system, lymphatic system, urinary system, reproductive system and alterations in metabolism and energetics.

Treatment of General Age-Related Degeneration

Methods and compositions of the present invention may be used to treat, ameliorate, reduce or compensate for general age-related degeneration. Similarly, methods and compositions of the present invention can be used to retain youthful functions of the body. Moreover, methods and products of the present invention may be used to treat specific age related system dysfunction, such as cognitive impairment, hearing loss, loss of visual activity, endocrine imbalances, skeletal changes and loss of reproductive function.

Cosmetic Use

In some embodiments, methods and compositions of the present invention may be used to prevent or reduce scars at a site of injury or infection. For example, cells or microRNAs may be employed to regenerate tissue that would otherwise scar or necrotize, including hepatic tissue in the treatment of hepatic fibrosis and/or cirrhosis, facial epidermal tissue to treat acne, and cardiac tissue in the treatment of ischemic infarction.

In some embodiments, methods and compositions (e.g., cells and/or microRNAs) according to the present invention may be used to enhance breast augmentation following mastectomy.

### IV. Pharmaceutical Compositions

In certain embodiments, the present invention provides pharmaceutical compositions comprising a therapeutically effective amount of cells and/or microRNAs for the treatment of various diseases, disorders or conditions described herein. Provided compositions typically comprise a therapeutically effective amount of cells (e.g., pathfinder cells, cells differentiated from pathfinder cells, and/or microRNAs associated with pathfinder cells) and a pharmaceutically acceptable carrier. In some embodiments, provided are pharmaceutical compositions comprising a therapeutically effective amount of cells and/or microRNAs for the treatment of an acute condition (e.g, myocardial infarct, renal ischemia, stroke, renal failure, liver failure, and complications of Type I diabetes). In some embodiments, provided are pharmaceutical compositions comprising a therapeutically effective amount of cells and/or microRNAs for alleviating transplantation stress. In some embodiments, provided are pharmaceutical compositions comprising a therapeutically effective amount of cells and/or microRNAs for the treatment of an immunodeficiency disease, disorder, or condition.

In certain embodiments, provided are pharmaceutical compositions comprising one or more microRNAs having a sequence at least 70% (e.g., 75%, 80%, 85%, 90%, 95%, 96%, 97%, 98%, 99%) identical to any of microRNAs identified in Table 1 and Tables 6-10 (e.g., SEQ ID NOs: 1-610, or SEQ ID NOS. 1-225) and a pharmaceutically acceptable carrier. As used herein, the term “pharmaceutically acceptable carrier” includes carriers that are approved by a regulatory agency of government or listed in the United States Pharmocopeia, the European Pharmocopeia, the United Kingdom Pharmocopeia, or other generally recognized pharmocopeia for use in animals, and in particular humans. As used herein, the term “carrier” refers to a diluent, adjuvant, excipient, or vehicle with which a therapeutic agent (e.g., cells and/or microRNAs) is administered.

Preparation of Compositions Comprising Cells

Various methods of preparing cells from biological materials and/or from cell cultures are known in the art and may be used to practice the present invention. In some embodiments, cells are enriched from their source material or culture. As used herein, the terms “isolation” or “isolating” in conjunction with cells are interchangeably used with the terms “enrichment” or “enriching,” and refer to one or more process steps that result in an increase of the fraction of cells in a sample or solution as compared to the fraction of cells in the material or culture from which it was derived.

Pathfinder cells (and/or cells that are differentiated from pathfinder cells) can be enriched or purified to homogeneity, to at least 90% (with respect to non-pathfinder cells), at least 80%, at least 70%, at least 60%, at least 50%, at least 40%, at least 30%, or at least 20% (or even less). For example, physical properties of pathfinder cells may be employed to separate them from a medium or other source material. For example, cells may be separated on the basis of size (e.g., filtration, molecular sieving, etc), density (e.g., regular or gradient centrifugation), or Svedberg constant (e.g., sedimentation with or without external force, etc). In some embodiments, cells are isolated or purified by centrifugation.

In some embodiments, the percentage of desired cells (e.g., pathfinder cells and/or cells differentiated from pathfinder cells) in a population of cells is assessed by methods based on one or more distinctive biological properties or set of biological properties of those cells. Especially suitable assessment methods may employ surface markers (e.g., for precipitation, reversible binding to solid phase, (fluorescence-activated cell sorting) FACS analyses or sorting, specific ligand binding, non-specific ligand binding such as annexin V, etc.). In some embodiments, cells having a particular set of characteristics (e.g., cell surface biomarker profile) are selected for using cell separation methods known in the art (e.g., marker-based cell separation methods). Thus, in some embodiments, a composition of cells comprising mostly or only cells of a desired type (e.g., pathfinder cells and/or cells differentiated from pathfinder cells) is obtained.

In some embodiments, cells are reconstituted in a suitable solution (such as pharmaceutically acceptable diluent or carrier) such that the cells are present in the solution at a known concentration. In some embodiments, the known concentration is chosen for convenience for dosing. In some embodiments, the known concentration is a concentration at which cells are known to be stable (e.g., retain viability and/or their therapeutic properties) for at least a period of time under clinically approved storage conditions. Provided compositions may also contain minor amounts of wetting agents, emulsifying agents, and/or pH buffering agents. Provided compositions can take any of a variety of solid, liquid, or gel forms, including solutions, suspensions, emulsions, tablets, pills, capsules, powders, sustained-release formulations, and the like. Non-limiting examples of suitable pharmaceutical carriers are described in “Remington's Pharmaceutical Sciences” by E. W. Martin. Compositions will generally contain a therapeutically effective amount of cells and/or microRNAs, optionally in purified form, together with a suitable amount of carrier so as to provide the form for proper administration to the patient.

Formulations are typically adapted to suit the mode of administration. For example, compositions for intravenous administration may be formulated as solutions in sterile isotonic aqueous buffer. Such compositions may also include a solubilizing agent and/or a local anesthetic such as lidocaine (also known as lignocaine, xylocalne, or xylocard) to ease pain at the site of injection.

As further example, compositions for topical and/or local use may be formulated, for example, as a lotion or cream comprising a liquid or semi-solid oil-in-water or water-in-oil emulsion and ointments. Such compositions may also comprise a preservative.

Compositions for delivery to the eye include may be formulated, for example, as eye drops that comprise the active ingredient in aqueous or oily solution and eye ointments that may be manufactured in sterile form. Compositions for delivery to the nose may be formulated, for example, as aerosols or sprays, coarse powders to be rapidly inhaled, or nose drops that comprise the active ingredient (e.g., cells and/or microRNAs) in aqueous or oily solution. Compositions for local delivery to the buccal cavity may be formulated, for example, as lozenges that comprise the active ingredient in a mass generally formed of sugar and gum arabic or tragacanth, and pastilles that comprise the active ingredient in an inert mass (for example of gelatine and glycerine or sugar and gum arabic). Flavoring ingredients may be added to lozenges or pastilles.

Aerosol and spray formulations may comprise, for example, a suitable pharmaceutically acceptable solvent (such as ethanol and water) or a mixture of such solvents. In some embodiments, such formulations comprise other pharmaceutical adjuncts (such as non-ionic or anionic surface-active agents, emulsifiers, and stabilizers) and/or active ingredients of other kinds Aerosol and spray formulations may be mixed with a propellant gas, such as an inert gas under elevated pressure or with a volatile liquid (e.g., a liquid that boils under normal atmospheric pressure below customary room temperature, for example from −30 to +10° C.).

Routes of Administration and Dosage Regimens

In methods of treatment or of inducing tissue repair, remodeling or differentiation in vivo of the present invention, cells, miRNAs, or a pharmaceutical composition thereof, will generally be administered in such amounts and for such a time as is necessary or sufficient to achieve at least one desired result. For example, cells or miRNAs can be administered in such amounts and for such a time that it amelioriates one or more symptoms of a disease, disorder, or condition; prolongs the survival time of patients; or otherwise yields clinical benefits.

A dosing regimen according to the present invention may consist of a single dose or a plurality of doses over a period of time. Administration may be, e.g., one or multiple times daily, weekly (or at some other multiple day interval), biweekly, monthly, or on an intermittent schedule. Typically an effective amount is administered. The effective amount of cells, microRNAs, or a pharmaceutical composition thereof, will vary from subject to subject and will depend on several factors (see below).

Cells, microRNAs, or pharmaceutical compositions thereof, may be administered using any administration route effective for achieving the desired therapeutic effect. Both systemic and local routes of administration may be used in accordance with methods of the invention. Suitable routes of administration include, but are not limited to, intravenous, intra-arterial, intramuscular, subcutaneous, cutaneous (e.g., topical), intradermal, intracranial, intrathecal, intrapleural, intra-orbital, intranasal, oral, intra-alimentary (e.g., via suppository), colorectal (e.g., via suppository), and intra-cerebrospinal.

Depending on the route of administration, effective doses may be calculated according to, e.g., the body weight and/or body surface area of the patient, the extent of damaged or diseased tissue, etc. Optimization of the appropriate dosages can readily be made by one skilled in the art, e.g., by a clinician. The final dosage regimen is typically determined by the attending physician, considering various factors that might modify the action of the cells, miRNAs, or pharmaceutical compositions thereof (collectively referred herein as “drug”), e.g., the drug's specific activity, the severity of tissue damage and the responsiveness of the patient, the age, condition, body weight, sex and diet of the patient, the severity of any present infection, time of administration, the use (or not) of other therapies, and other clinical factors.

In some embodiments, dosing is given by number of cells per body weight of the individual to which cells are administered. In some embodiments, approximately 1×10⁶ to 3×10⁸ cells per kg per dose are administered. In some embodiments, approximately 1×10⁶ to approximately 3×10⁸, approximately 1×10⁷ to approximately 3×10⁸, or approximately 3×10⁷ to approximately 3×10⁸ cells are administered per kg per dose. In some embodiments, dosing is not calculated based on body weight and/or dosing is not calculated based on number of cells administered. For example, in some embodiments, a standard unit may be developed based on effectiveness in a particular measure for a particular condition. Alternatively or additionally, in some embodiments, recommended dosages are developed for adult and/or child individuals without regard to body weight.

### EXEMPLIFICATION

### Example 1

### Stimulation of Repair by Pancreas-Derived Pathfinder Cell (PDPC) in a Kidney Ischemic Damage Model

The present Example demonstrates that pancreas-derived pathfinder cells (PDPCs) can stimulate repair in a model of renal ischemic reperfusion injury that has direct relevance to acute renal failure and decreased allograft survival in the context of kidney transplantation. (See, e.g., Hochegger et al. (2007) “p21 and mTERT are novel markers for determining different ischemic time periods in renal ischemia-reperfusion injury,” Am. J. Physiol. Renal Physiol., 292:762-768, the entire contents of which are herein incorporated by reference.) In this well-accepted model of renal cell damage, the extent of tissue damage depends on the length of the ischemic period.

### Materials and Methods

Renal Ischemia Model

Renal ischemia was induced in mice using protocols as previously described (Hochegger et al. (2007)). C57BL/6 male mice were maintained on a standard diet with water available ad libitum. Mice were anesthetized and an incision was made on the central abdomen. Clamps were applied to bilateral renal pedicles. After 20 or 30 minutes of renal ischemia, clamps were removed, and the incision was closed.

Transplantation of Rat PDPCs

PDPCs were isolated and cultured from male rats as previously described, for example, in International Patent Publications WO 2006/120476 and WO 2009/136168, the entire contents of each of which are incorporated by reference. Rat PDPCs were counted and reconstituted in phosphate-buffered saline solution (PBS) and injected into the tail vein of mice after renal ischemia. Varied cell treatment regimens were used depending on the experiment. (See below.)

Control animals were given an injection of saline or an equivalent number of C57BL/6 bone marrow cells.

Assessment of Renal Function

Renal function was monitored for two weeks after the ischemic event by determining serum creatinine levels and the urinary protein to creatinine ratio (UPCR). Increased levels of these markers correspond to increased extent of tissue damage. Serum and urine samples were collected at two weeks after renal ischemia. Creatinine levels were measured using a creatinine analyzer and UPCR was determined by standard methodology.

Kidney Histology

Animals were sacrificed at the end of the experiment for histological analyses to assess extent of tubular epithelial necrosis and tissue senescence. Kidney tissue was either frozen or fixed in 4% formalin overnight and then embedded in OCT embedding medium. Paraffin-embedded kidneys were sectioned and stained with periodic acid-Schiff or hematoxylin and eosin using standard protocols. To assess tubular injury in periodic acid-Schiff-stained sections, the percentage of cortical tubules was scored by a reviewer blind to the identity of each sample, using a semiquantitative scale as previously described. (See, e.g., Ramesh et al. (2004) “Inflammatory cytokines in acute renal failure,” Kidney Int. Suppl., S56-S61 and Ramesh et al. (2004) “Salicylate reduces cisplatin nephrotoxicity by inhibition of tumor necrosis factor-alpha,” Kidney Int., 65:490-499, the entire contents of each of which are herein incorporated by reference.) In the semiquantitative scoring system, 0=no tubular necrosis; 1=<10%; 2=10-25%; 3=26-75%; 4=>75% tubular necrosis.

Analysis of Apoptosis

Apoptosis was evaluated by TUNEL staining of kidney sections as follows using a Chemicon ApopTag Plus Peroxidase In Situ Apoptosis Detection Kit (S7101). Frozen kidney sections were fixed by incubating slides in 95% ethanol for 10 minutes and then allowed to air dry. Slides were stored at 4° C. until further use. Sections were rehydrated at room temperature in DPBS for 10 minutes and then digested by incubation in Proteinase K (20 μg/mL) at 25° C. for 15 minutes, then washed twice in dH₂O for 2 minutes each.

Sections were treated with H₂O₂ for 5 minutes at room temperature on a stirrer, washed in 1×TBS (Tris-buffered saline), and then incubated in equilibration buffer at 25° C. for 10 minutes in a humidified chamber. After removal of equilibration buffer, sections were incubated in a TdT enzyme solution (Chemicon; Rosemont, Ill.) at 37° C. for 1 hour in a humidified chamber. Slides were placed in a stop/wash buffer and agitated for 15 seconds, then washed for 10 minutes on a stirrer at room temperature. Slides were washed in 1×TBS, then incubated in a solution of anti-digoxygenin conjugate (Chemicon), then washed in 1×TBS. Sections were incubated in a peroxidase substrate solution (Chemicon), and incubated at room temperature for 10 minutes, then washed in running water for ten minutes. Finally, sections were counterstained in Harris hematoxylin, dehydrated, and mounted using standard protocols.

Analysis of Markers of Cell Stress and Senescence

Cell stress and senescence was analyzed by immunohistochemical staining for markers of cell stress and senescence (CDKN1A (p21cip1/waf), CDKN2A9 (p16ink4a), and SAβgal). Tissue sections were fixed in 95% ethanol and rehydrated as described above, and then stained using appropriate reagents for each markers.

For p16ink4a staining, a mouse monoclonal antibody against p16 (F12, Santa Cruz sc#1661) was used as the primary antibody and an anti-mouse secondary antibody was used. To reduce binding of the anti-mouse secondary antibody to endogenous mouse immunoglobulins in the tissue, the Vector Mouse on Mouse (MOM) Peroxidase Kit (Vector #Pk-2200) was used. A biotinylated anti-mouse IgG (H+L) antibody from Vector MOM kit was used as the secondary antibody. Immunohistochemical staining was performed according to the Vector protocol for the MOM kit.

For p21cip1/waf staining, a rabbit polyclonal antibody against p21 (C19, Santa Cruz sc#397) was used as the primary antibody and a horseradish peroxidase (HRP)-conjugated goat anti-rabbit IgG secondary antibody (DAKO, P0448) was used. Twenty percent goat serum prepared in 1×TBS was used as blocking solution to reduce background staining from the secondary antibody. Staining was performed according to standard procedures.

For SAβgal staining, two solutions containing final concentrations of 1 mg/mL xl-Gal, 5 mM potassium ferricyanide, 5 mM potassium ferrocyanide, 2 mM MgCl₂, 150 mM NaCl, and 40 mM (citric acid/sodium phosphate solution) were prepared in phosphate-buffered solution (PBS), one using a citric acid/sodium phosphate solution at pH 6.0 and the other using a citric acid/sodium phosphate solution at pH 4.0. Sections were stained in the pH 6.0 solution (except for positive controls, which were stained with the pH 4.0 solution) at 37° C. for approximately 48 hours. Sections were washed in running water and counterstained in a filtered Aluminum Potassium Sulphate Dodecahydrate (50 g/L) and Nuclear Fast Red (1 g/L) solution for 30 seconds, then washed, dehydrated, and mounted according to standard procedures.

Fluorescence In Situ Hybridization (FISH)

Frozen sections were rehydrated for 20 minutes in PBS and incubated in 1 M sodium thiocyanate at 80° C., then washed TBS and dried. Tissue sections were then digested in pepsin (DAKO; Carpenteria, Calif.) at 37° C. for 25 minutes. Pepsin was quenched in 0.2% glycine for 5 minutes at room temperature. Slides were washed in TBS and then incubated in 4% paraformaldehyde for 5 minutes at room temperature. After checking digestion by pepsin, denaturation, and dehydration, sections were incubated with either a probe solution containing hybridization buffer (Vysys #30-804828) and a denatured probe for the human X chromosome (VYSIS CEP X FITC probe #32-112023) or a premixed solution (available from the manufacturer as such) containing a denatured probe for the rat Y chromosome (Cambio Rat Y (Cy3)/12) FITC #CA-1631-BF) for 10 minutes at 80° C., then overnight at 42° C. Slides were then washed, mounted, and coverslipped using standard procedures. Coverslips were sealed with clear nail polish.

### Experiment 1

A small-scale preliminary experiment was carried out using 2 ischemic controls and 2 rat PDPC-treated mice. Ischemia was carried out for 20 minutes to mimic a conservative damage level, and rat PDPCs were delivered 24 hours after ischemic damage. 1.5×10⁶ cells were used for each injection, with one injection given at 24 hours post ischemia and second injection given 7 days post ischemia. Data from this preliminary experiment were suggestive of a beneficial effect from PDPCs. Although no significant improvement in serum creatinine levels were observed at 1 week after surgery, the UPCR was decreased in PDPC-treated mice, indicating less tubular epithelium damage. Decreased damage was confirmed by histological analyses.

### Experiment 2

The surprising positive results from the preliminary experiment prompted a fuller experiment in a second study. For this second study, renal ischemic mice were assigned into one of four groups of six mice each. In addition to control ischemic mice injected with saline only, groups of mice were subject to various treatment regimens as follows.

Group A: Saline only injection at 24 hours after ischemia.

Group B: 1.5×10⁶ rat PDPCs were injected 24 hours after ischemia and again at 7 days

Group C, 1.5×10⁶ rat PDPCs were injected immediately after ischemia and again at 7 days

Group D: 3×10⁶ rat PDPCs were injected 24 hours after ischemia.

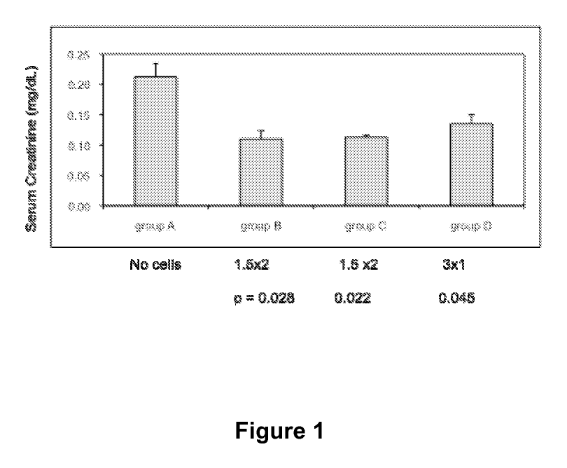

Experiments were terminated at 14 days after ischemia. All three the PDPC treatment regimens resulted in statstically significant benefits for maintenance of kidney function and tissue integrity. As shown in FIG. 1, serum creatinine levels at day 14 were significantly lower in all treated groups (p=0.028, 0.022, and 0.045 respectively) as compared to that of saline-injected ischemic controls. No significant differences were observed between serum creatinine levels of the three treatment groups.

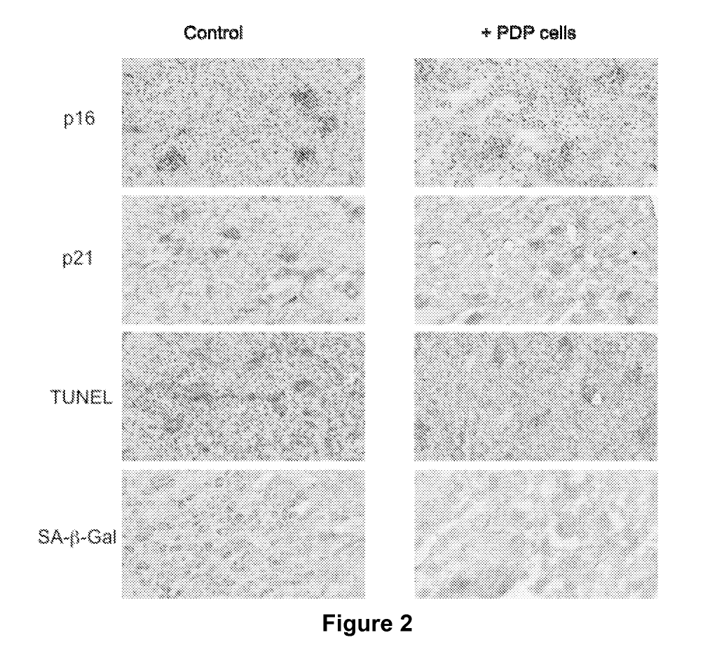

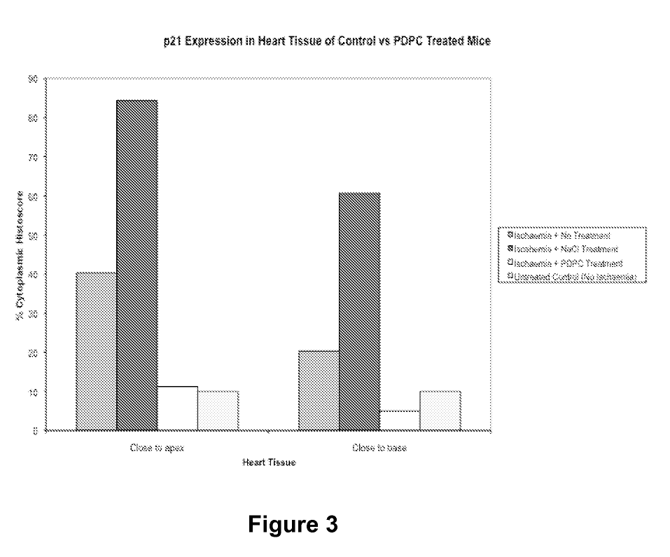

Apoptosis in kidneys were evaluated by TUNEL-staining of tissue sections. Virtually no apoptosis was observed in kidney tubular epithelium of PDPC-treated mice, whereas saline-injected controls exhibited high levels of apoptosis. p21cip1/waf, p16ink4a, and SAβgal expression was used as a marker of cell stress and senescence. As determined by immunohistochemical staining, expression of these three markers was reduced in kidney tissues of PDPC-treated mice compared to ischemic controls, indicative of lower levels of cell stress and senescence (FIG. 2). FIG. 3 shows a quantitative results of analysis of p21 expression levels measured in tissue taken from two areas of the heart. Animals with ischemia and no treatment (left-most bars in each group in FIG. 3) showed an increased level of p21, which was made worse by saline injection (second-left most bars in each group in FIG. 3). PDPC-treated animals had p21 levels that were essentially the same as animals without any ischemia.

The fate of PDPC cells in mouse transplant recipients was analyzed by FISH, which revealed that approximately 0.019% of cells in the kidney are PDPC-derived (54 rat signals in 281,320 nuclei examined in tissue from a mouse in group B). Thus, PDPC-derived cells are still present, but make up only a small proportion of the tissue.

### Experiment 3

In a further study, renal ischemic mice were assigned into one of four groups. In addition to control ischemic mice injected with saline only, groups of mice were subject to various treatment regimens as follows.

Group A: Saline only injection at 24 hours after ischemia (6 mice).

Group B: 1.5×10⁶ rat PDPCs were injected 24 hours after ischemia and again at 7 days (5 mice).

Group C: 3×10⁶ rat PDPCs were injected 24 hours after ischemia (3 mice).

Group D: 1.5×10⁶ rat PDPCs were injected immediately after ischemia and again at 7 days (4 mice).

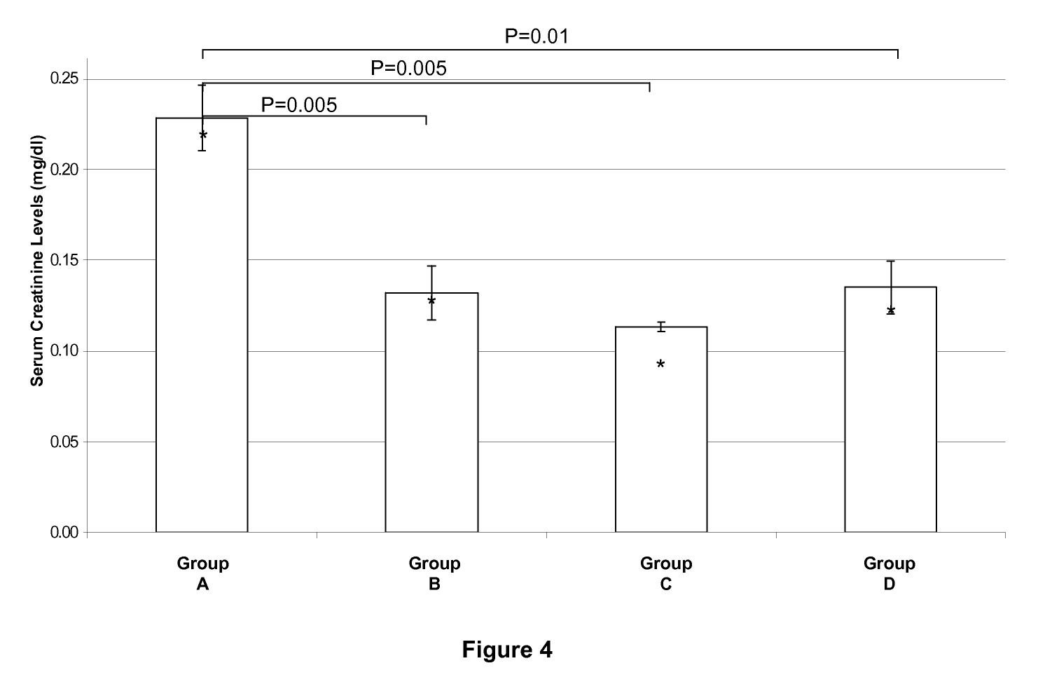

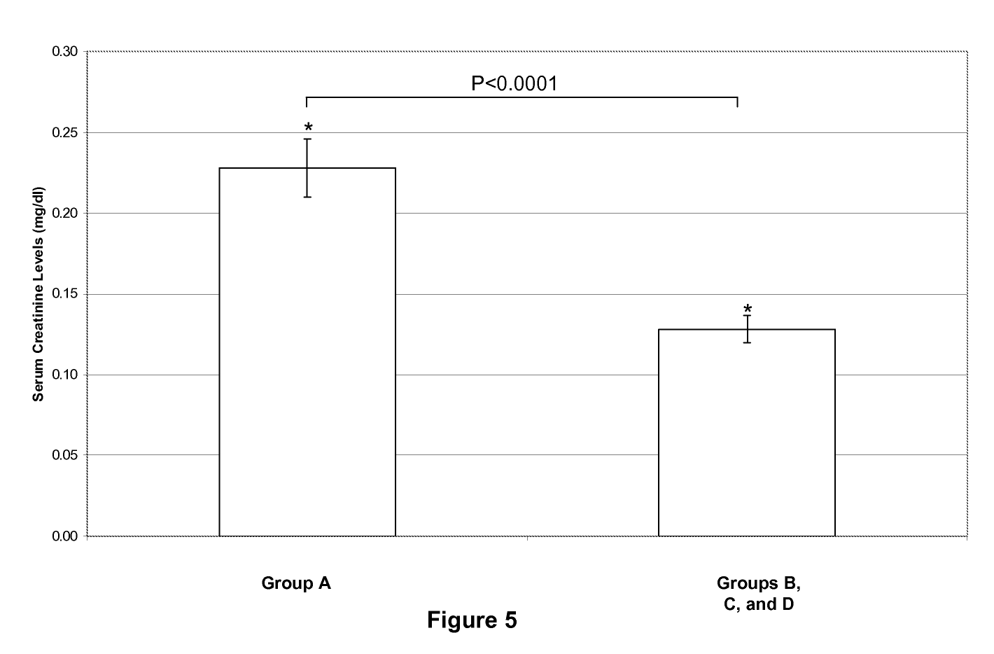

Experiments were terminated at 14 days after ischemia. All three the PDPC treatment regimens resulted in statstically significant benefits for maintenance of kidney function and tissue integrity. As shown in FIG. 4, serum creatinine levels at day 14 were significantly lower in all treated groups (p=0.005, 0.005, and 0.01 respectively) as compared to that of saline-injected ischemic controls. No significant differences were observed between serum creatinine levels of the three treatment groups. FIG. 5 shows serum creatinine levels for untreated control mice (Group A, n=6) as compared to mice treated with rat PDPCs (n=12). As can be seen in FIG. 5, serum creatinine levels at day 14 were significantly lower in groups B, C, and D combined (p<0.0001) as compared to that of saline-injected ischemic controls

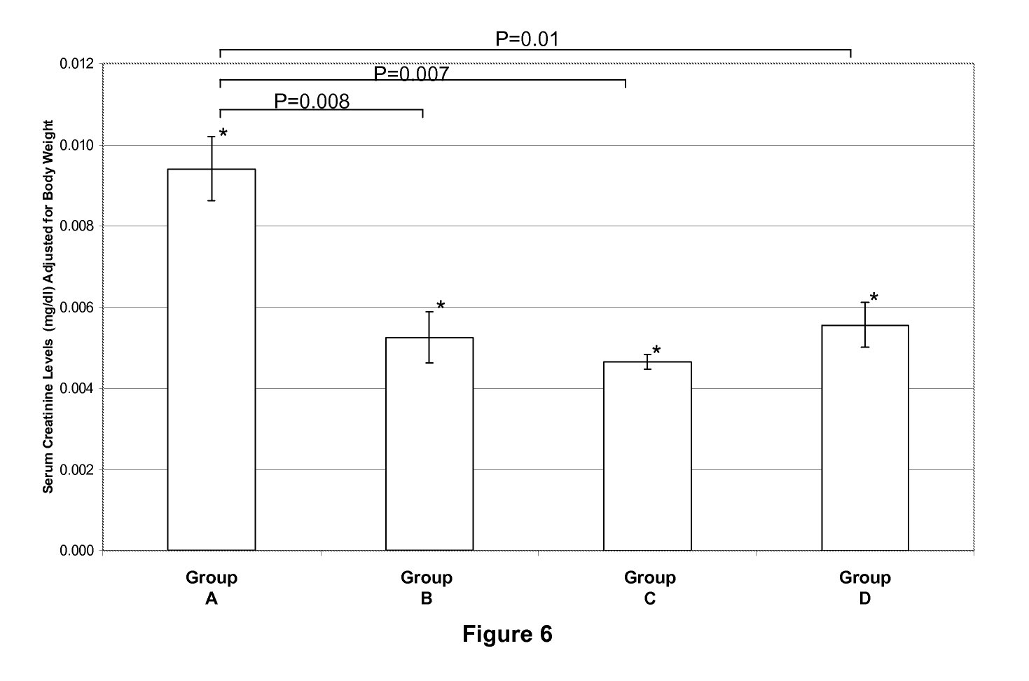

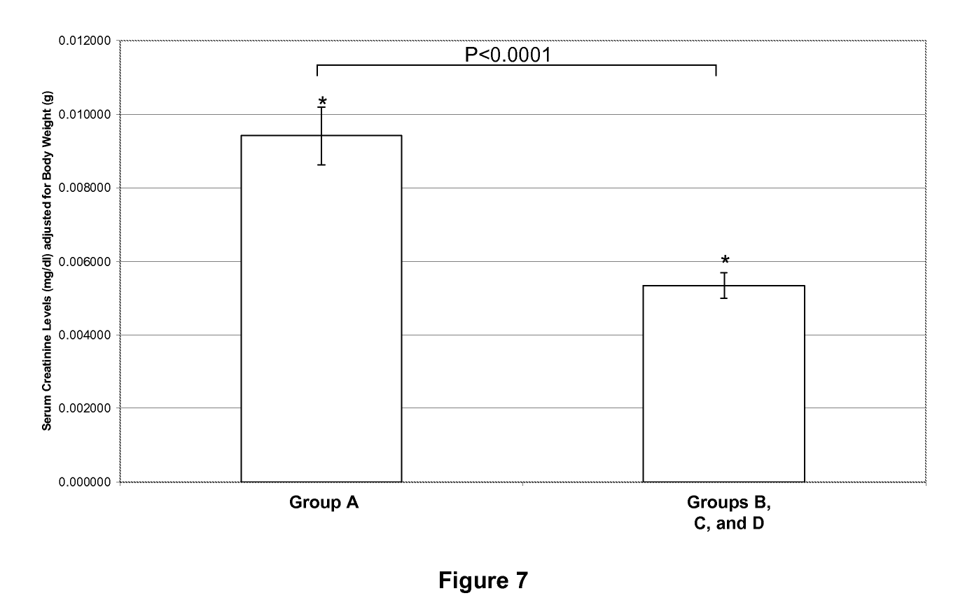

Serum creatinine levels adjusted for body weight were determined. As shown in FIG. 6, serum creatinine levels adjusted for body weight at day 14 were significantly lower in all treated groups (p=0.008, 0.007, and 0.01 respectively) as compared to that of saline-injected ischemic controls. No significant differences were observed between serum creatinine levels adjusted for body weight of the three treatment groups. FIG. 7 shows serum creatinine levels adjusted for body weight for untreated control mice (Group A, n=6) as compared to mice treated with rat PDPCs (n=12). As can be seen in FIG. 7, serum creatinine levels adjusted for body weight at day 14 were significantly lower in groups B, C, and D combined (p<0.0001) as compared to that of saline-injected ischemic controls

Taken together, these results demonstrate that Pathfinder cells can stimulate repair in a tissue that is not the same as the tissue of origin of Pathfinder cells. Furthermore, Pathfinder cells can alleviate acute damage to kidneys that have undergone ischemic stress. These findings support a potential for Pathfinder cells in treating acute conditions such as acute kidney damage and in alleviating transplantation stress.

### Example 2

### Stimulation of Repair by PDPCs in a Cardiac Ischemic Damage Model

The present Example demonstrates that pancreas-derived pathfinder cells (PDPCs) can stimulate repair in an established model of cardiac ischemic damage with directed relevance to acute myocardial infarction (MI). (See, e.g., Metzler et al. (2002) “Plasma cardiac troponin T closely correlates with infarct size in a mouse model of acute myocardial infarction,” Clinica Chimica Acta, 325:87-90, the entire contents of which are herein incorporated by reference.) The level of plasma cardiac troponin T (cTnT) after the surgery performed to induce ischemia has been shown to be proportional to the size of the induced infarct. In this model, the extent of tissue damage depends on the length of the ischemic period.

### Materials and Methods

Cardiac Ischemia Model

Cardiac ischemia was induced in mice using protocols as previously described (Metzler et al. (2002)). C57BL/6 mice were anesthetized. A midline incision was made from the xiphoid process to the submentium. After separating the salivary glands, a tracheotomy was performed by inserting a polyethylene tube carefully into the trachea. The tube was taped in place to prevent dislodgment and connected to a rodent ventilator. After ventilation was started, the chest was opened by a lateral cut along the left side of the sternum. Large intercostal blood vessels were coagulated using an electrocautery. The chest walls were retracted to better visualize the heart. After the pericardial sac was removed and the left auricle was slightly retracted, the left descending artery (LAD) was clearly visible. The LAD was ligated using 8-0 silk; ischemia was evident from discoloration of corresponding regions of the left ventricle. The LAD was left ligated for 30 minutes, after which refraction sutures were removed and the pericardium, chest wall, and skin were closed with sutures.

Transplantation of Rat PDPCs

PDPCs were isolated from rats and transplanted into mouse hosts as described in Example 1.

Assessment of Cardiac Function

Cardiac function was assessed before termination of the experiment, at 14 days after ischemia. In vivo ultrasound imaging of systolic left ventricle was used to determine left ventricle size and fractional shortening (FS). A smaller left ventricle size corresponding to less muscle damage and to better function. FS represents the ratio of heart size when full with blood (LVEDD, left ventricle diastolic diameter) to heart size after emptying (LVESD, left ventricle systolic diameter), which reflects how much the heart muscle needs to contract to eject a normal volume of blood. Increased fractional shortening corresponds to increased heart function (with 50% being a normal measurement).

Cardiac function was also assessed by taking and analyzing electrocardiograms.

Cardiac Troponin (cTnT) Measurements

Cardiac troponin (cTnT) levels in plasma were measured weekly using protocols as described in Metzler et al. (2002). A quantitative rapid cTnT assay available from Roche Diagnostics (Cardiac T, Troponin T quantitative, Cardiac Reader) was used according to manufacturer's instructions.

Histology

Post-mortem histological analysis of the heart was conducted using standard protocols similar to those described for kidney histology in Example 1. Histological analyses focusing on the size of infract and residual scarring were performed as described in Metzler et al. (2002).

Fluorescence In Situ Hybridization (FISH)

FISH was conducted to examine the number, location, and nature of rat cells remaining and the extent and location of any senescent tissue using protocols as described in Example 1.

### Experiment 1

A small-scale pilot experiment was conducted with three mice injected intravenously with 1.5×10⁶ rat PDPCs 24 hours after surgery. Clear benefits were observed as compared to ischemic controls that were injected with saline. Cardiac function in one PDPC-treated mouse returned almost to normal.

### Experiment 2

The surprising successful results from the pilot experiment prompted a fuller experiment in a second study. For this second study, mice in which cardiac ischemia was induced were assigned to one of three groups as follows:

Group A: No treatment (4 mice)

Group B: Saline injection at 24 hours after infarct induction (6 mice)

Group C: 1.5×10⁶ PDPCs were injected 24 hours after infarct induction (6 mice)

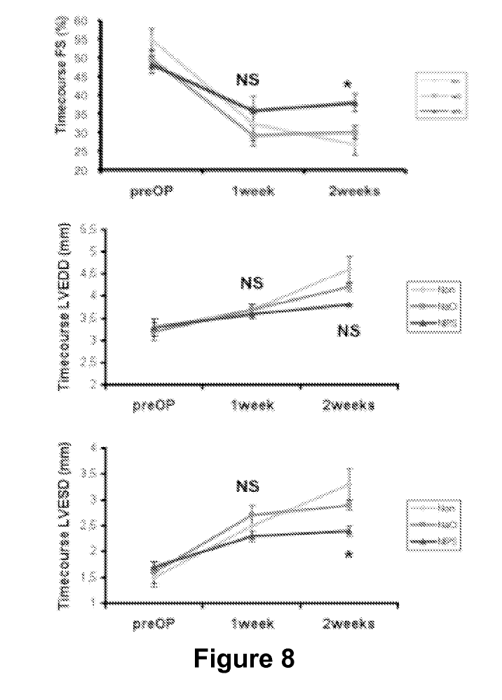

cTnT levels were not significantly different between the treatment groups, indicating that infarct sizes were comparable across all ischemic mice. Cardiac function was assessed two weeks after surgery. As determined by analyses on 4 of the 6 PDPC-treated mice, FS (fractional shortening) and LVESD (left ventricle systolic diameter) data showed statistically significant improvements in cardiac function after PDPC treatment, and there was likewise a clear trend in the LVEDD data (FIG. 8). The p-value for the difference in LVESD in PDPC-treated versus no treatment controls is at the 0.016 level (0.047 with Bonferroni correction). The p-value for the difference in LVESD in PDPC-treated versus saline treated groups is at the 0.032 level (0.096 with Bonferroni correction). Analyses including all 6 mice in the PDPC-treated group is expected to show p-values consistently less than 0.05.

### Experiment 3

In a further study, mice in which cardiac ischemia was induced were assigned to one of three groups as follows:

Group A: No treatment (5 mice)

Group B: Saline injection at 24 hours after infarct induction (10 mice)

Group C: 1.5×10⁶ PDPCs were injected 24 hours after infarct induction (6 mice)

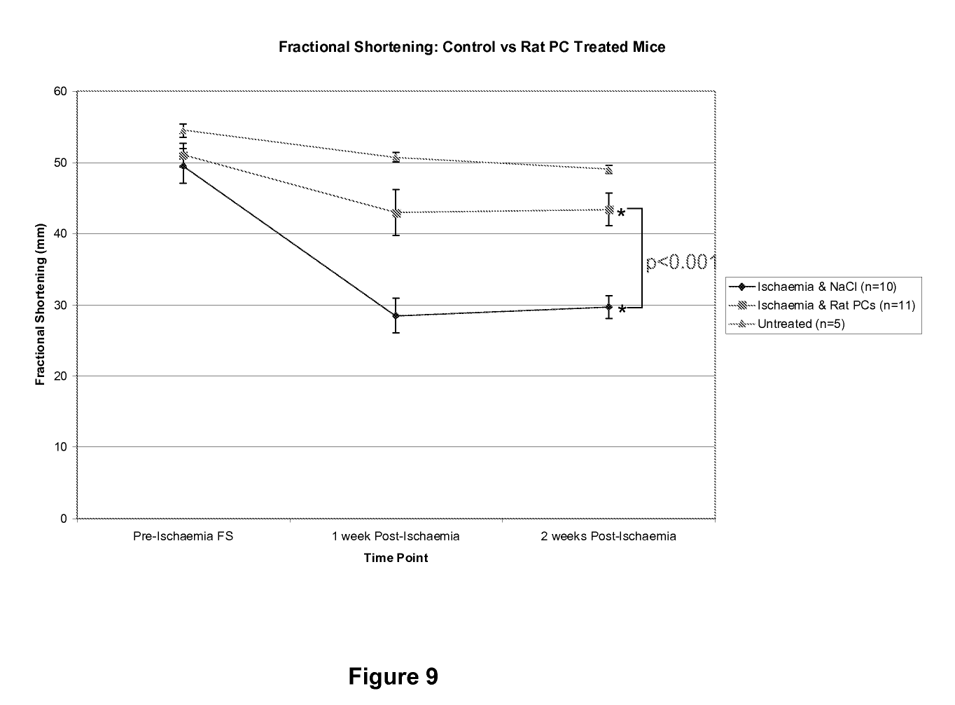

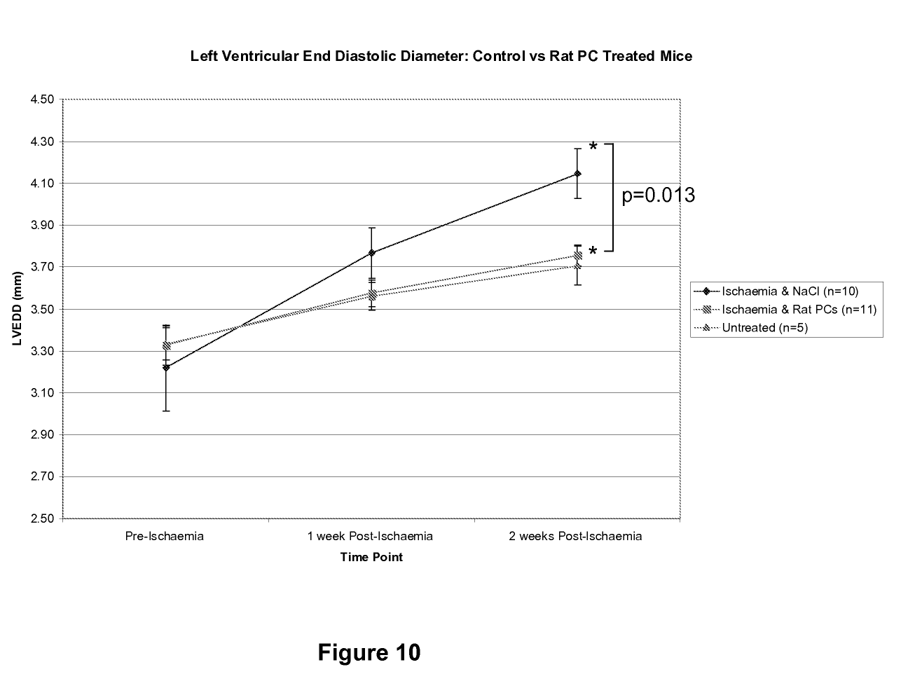

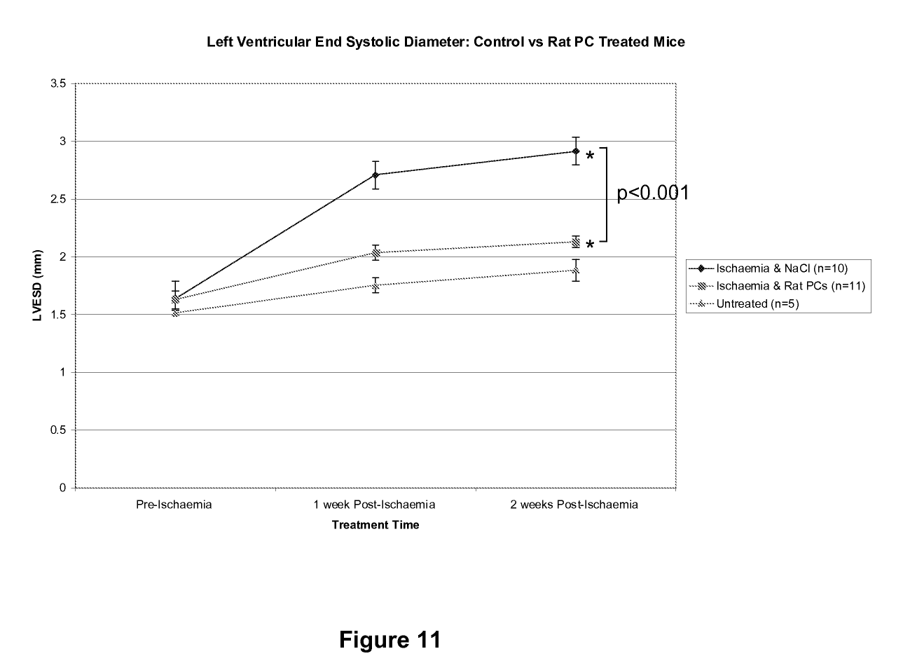

cTnT levels were not significantly different between the treatment groups, indicating that infarct sizes were comparable across all ischemic mice. Cardiac function was assessed two weeks after surgery. As determined by analyses of PDPC-treated mice, FS (fractional shortening), LVEDD (left ventricle diastolic diameter), and LVESD (left ventricle systolic diameter) data showed statistically significant improvements in cardiac function after PDPC treatment (FIGS. 9, 10, and 11, respectively). The p-value for the difference in FS in PDPC-treated versus no treatment controls was <0.001. The p-value for the difference in LVEDD in PDPC-treated versus saline treated groups was 0.013. The p-value for the difference in LVESD in PDPC-treated versus saline treated groups was <0.001.

Typically, recovery is considered successful when patients achieve an FS of over 30% (such as 35%). PDPC-treated mice attain levels of FS well within the range regarded as normal after clinical recovery in human patients, namely up to 40-45%. Thus, these results support a possible therapeutic application of Pathfinder cells in treating acute myocardial infarct.

### Example 3

### Reconstitution of Bone Marrow by PDPCs in a Post-Irradiation Reconstitution Model

The present Example demonstrates that pancreas-derived pathfinder cells (PDPCs) can stimulate reconstitution of the blood system.

### Preliminary Experiment

A preliminary set of experiments used the NOG multiply immunodeficient strain (non obese diabetic/severe combined immunodeficient/gammac (null); NOD/SCID/Gammac). (See, e.g., Dewan et al. (2003) “Rapid Tumor Formation of Human T-Cell Leukemia Virus Type 1-Infected Cell Lines in Novel NOD-SCID/γcnull Mice: Suppression by an Inhibitor against NF-κB,” Journal of Virology, 77(9):5286-5294, the entire contents of which are herein incorporated by reference.) Irradiated NOG mice would not normally be able to reconstitute their blood system.

NOG mice were irradiated at a level to which they would not normally recover (3.5 Gy). Three irradiated NOG mice were administered 1.5×10⁶ PDPCs intravenously (by tail vein injection-yes) immediately after irradiation; two of these mice received rat PDPCs and one of these mice received human PDPCs. Three irradiated NOG mice were given saline as controls.

All three control mice died within two weeks, while all three mice that received PDPCs lived well beyond the survival time until they contracted a laboratory virus infection at beyond 56 days post-irradiation and died. These results demonstrated that PDPC-treated mice were able to produce blood cells and reconstitute their blood system.

It was not possible to obtain high quality DNA from the mouse carcasses. Nevertheless, the DNA that could be obtained was assayed by PCR for rat Y and human X chromosomes. In these assays, no rat signal was detectable, and only a weak human signal was found in the spleen of one carcass. On the other hand, the control mouse actin gene was detectable. These results suggest that the reconstituted blood cells were at least mostly of host (and not PDPC) origin.

Survival of the PDPC-treated mice to at least 56 days also suggested a lack of tumor formation from the introduction of rat or human PDPCs.

### Further Experiments

A further set of experiments is performed on male NOD/SCID (non obese diabetic/severe combined immunodeficient) mice. Experimental groups include:

Group A: a control group of unirradiated mice, injected with saline

Group B: a control group of irradiated mice, injected with saline

Group C: a control group of irradiated mice, administered 1.5×10⁶ mouse NOD/SCID bone marrow cells

Group D: an experimental group of irradiated mice, administered 1.5×10⁶ rat PDPCs.

The experiment is terminated at a definitive timepoint (e.g., 14 days or later) after injection, or when a mouse's body weight falls under 40% 50% of body weight at the beginning of the experiment. Survival time is noted and used as an indicator of reconstitution of the blood system. Blood samples are collected and analyzed by FACS for rat-specific blood cell markers to determine the extent, if any, of contribution from rat PDPCs to the reconstituted blood cell population. Additionally FACS analyses and/or PCR analyses may be used to (1) determine cell origin and/or (2) verify production of functional blood cells.

It is expected that results from these further experiments will confirm positive results from the preliminary experiments.

These results indicate that pathfinder cells can reconstitute blood system.

Examples 1-3 demonstrate that PCs enhance the survival and repair of various tissues in several in vivo models of acute damage in various tissues. No tumors formed in any of the models. The data thus far indicate that PCs themselves do not contribute a significant percentage of the repaired tissue population, but rather, stimulate repair of damaged host cells.

### Example 4

### Efficacy of Kidney-Derived Pathfinder Cells (KDPCs) in a Streptozocin (STZ)-Induced Diabetes Model

Previously reported results demonstrated that PDPCs exhibited a therapeutic effect in a chemically-induced (streptozocin, STZ) model of diabetes. (See International Patent Publication No. WO 2006/120476.) The present Example demonstrates that kidney-derived Pathfinder cells (KDPCs) similarly show repair stimulatory effects in the STZ-induced model of diabetes.

Human KDPCs were isolated by a procedure similar to that described for PDPCs in International Patent Publication No. WO 2006/120476.

Female mice were made diabetic by administration of 250 mg/kg streptozocin (STZ). Blood glucose was monitored to confirm the animals were diabetic, with levels increasing to above 15 mM/L. Three days after induction of diabetes, 1.5×10⁶ human KDPCs were administered by injection into the tail vein. Control animals were given saline injections. Blood glucose was monitored every three days thereafter. A second injection of the same number of KDPCs was administered seven days later and blood glucose was again monitored every three days thereafter.

Control animals (n=2) rapidly showed elevated blood glucose levels to over 30 mM/L and died within 6 days post injection. STZ diabetic mice (n=2) that received human kDPCs showed initial elevation in blood glucose levels, then stabilization of blood glucose levels around 25 mM/L, as shown in Table 2. Body weight was maintained until the two test animals were reported found dead at day 14. Maintenance of body weight indicates that the test animals were otherwise healthy.

TABLE 2
Blood glucose levels in control animals and KDPC-treated mice

Time (days)

| | | | | | |
|---|---|---|---|---|---|
| 0 | 3 | 6 | 9 | 12 | 14 |

Blood glucose mM/L

| | | | | | | |
|---|---|---|---|---|---|---|
| hKDPC 1 | 5.3 | 21 | 30.6 | 21.3 | 26 | died |
| hKDPC 2 | 8.1 | 25.9 | 21.3 | 25.1 | 24.6 | died |
| Control 1 | 5 | 30.3 | died | | | |
| Control 2 | 8.7 | 31.9 | died | | | |

The survival of test animals beyond the survival of controls, and the relative stabilization of blood glucose levels similar to previously reported levels of PDPC-treated animals at the same timepoint after induction of diabetes, suggested that administration of human KDPCs arrested or ameliorated the diabetic process. Thus, KDPCs appear to act similarly to PDPCs, indicating that PCs are not restricted to the pancreas. Thus, it is contemplated that KDPCs would show similar repair stimulatory effects in acute models of damage as PDPCs have, and would be useful in reconstituting an immune system.

### Example 5

### PDPC Effects in the Non-Obese Diabetic (NOD) Mouse Type I Diabetes Model

As mentioned herein, methods and compositions of the present invention can be used to treat acute complications of chronic diseases. The present Example demonstrates that PDPCs can stabilize glucose blood levels in non-obese diabetic (NOD) mice treated with PDPCs.

The NOD model is widely accepted as a much closer model of human Type I diabetes than chemical damage models such as STZ (streptozocin). The NOD mouse also allows further investigation of how repair-stimulatory effects of pathfinder cells can be established and maintained in the face of pre-existing immune attack.

NOD mice become spontaneously diabetic as they age. Diabetes can also be induced. In the present Example, diabetes was allowed to develope spontaneously. Animals were entered into a trial with rat PDPC delivery as they became diabetic. The trial is currently underway.

NOD mice were divided into three groups (1) untreated, (2) treated with PDPCs, and (3) treated with PDPCs and cyclosporin A (CsA, an immunosuppressant). Each group consisted of six mice each. In groups (2) and (3), PDPCs were administered intravenously by tail vein injection. CsA was also administered for group (3) animals at the time of PDPC administration.

As the trial is underway, some mice in Group I are still in the study and do not yet show elevated glucose levels (indicating development of diabetes). The other mice in the study (including all mice in groups 2 and 3) developed diabetes according to one of two patterns: intense hyperglycemia with rapid onset or milder hyperglycemia with slower onset.

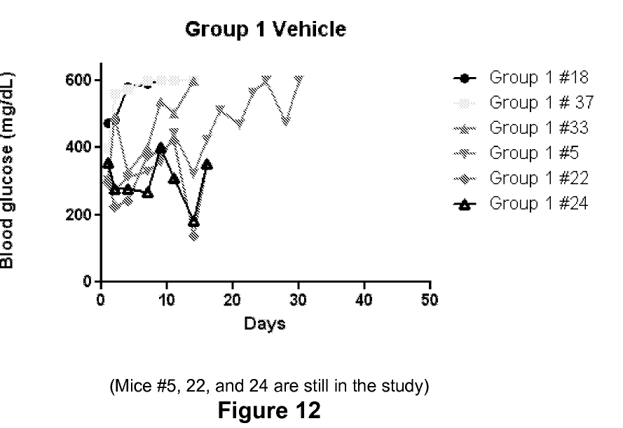

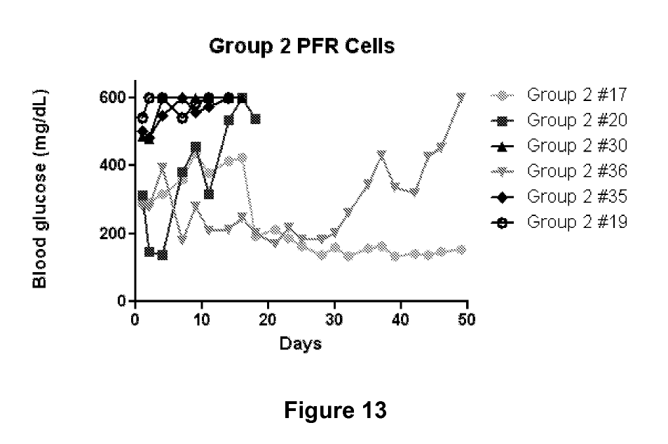

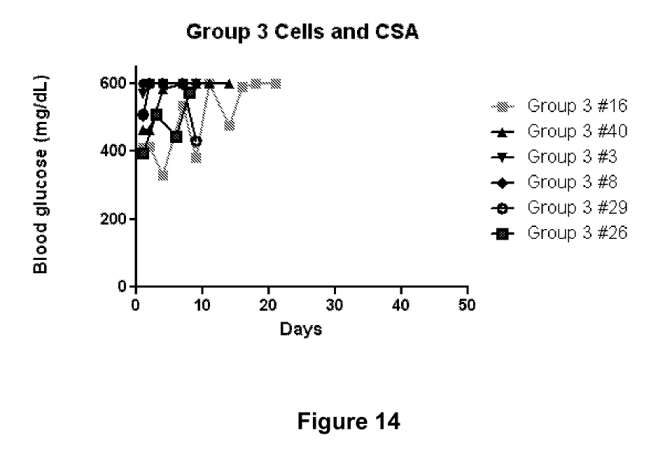

Thus, some of the mice in this study present an opportunity to observe PDPC effects in the context of acute onset of hyperglycemia. FIGS. 12-14 show blood glucose levels in groups 1, 2, and 3 respectively. As shown in FIG. 13, two of the mice treated with PDPCs (group 2) showed a lowering of blood glucose levels. One of these mice maintained low blood glucose levels until necropsy at day 50; the other of these mice also showed a lowering of blood glucose levels until day 30 of the study, followed by onset of hyperglycemia. No efficacy was observed in group 3 (cells+CsA) animals.

### Example 6

### Morphological Examination of Pancreas-Derived Pathfinder Cells (PDPC) and Identification of Microvesicles (MVs)

In the present Example, morphological studies of rat pancreas-derived pathfinder cells (PDPC) were conducted by scanning electron microscopy (EM). Scanning EM images revealed protrusions from surfaces of PDPCs that are provisionally identified as nascent microvesicles (MVs).

Pathfinder cells were isolated from rat pancreas cultured as previously described. (See, e.g., International Patent Publication No. WO2006/120476 A1, the entire contents of which are herein incorporated by reference.) These rat PDPCs were grown in medium containing fetal bovine serum (FBS) that was depleted of bovine microvesicles.

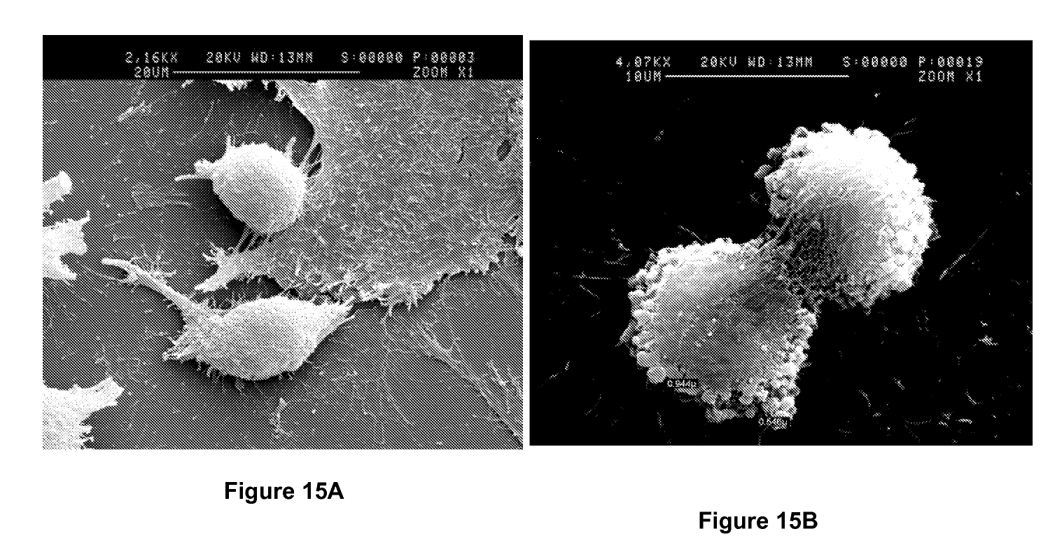

Pictures of a subconfluent culture of rat PDPCs were taken by a scanning electron microscope. FIG. 15A shows a representative picture, showing PDPCs of both the fibroblastoid and small round cell types. As can be seen in FIG. 15A, both cell types have very great numbers of thin projections and interconnect with other cells at multiple points in a complex manner. Furthermore, these cells produce large numbers of small spheres on their surfaces, which are identified as nascent microvesicles (FIG. 15B).

The flat cell type depicted in FIG. 15A is approximately 15-20 μm in diameter, and is the predominant cell type in cultures that were studied. The other cell type is approximately 3-5 μm in size, spherical in morphology, and is commonly found adjoined to an identical cell type. Without wishing to be bound by any particular theory, these spherical cells may be derived from a cell that has recently undergone cell division.

Protrusions of varying length can be seen radiating from the edges of the flatter, larger cell type in particular. Putative microvesicles (MVs) were clearly observed at the ends of these cell protrusions. In some cases, the MVs were not actually attached to the cells but were still within the vicinity of cells and of attached MVs. MVs were also clearly seen close to and surrounding the membrane of the small cell type (FIG. 15B). Clusters of MVs were observed in some areas, typically at the end of a cell protrusion. Identified MVs typically had a size range of 300-600 nm in diameter.

### Example 7

### Characterization of Protein and mRNA Profiles of Pathfinder Cell Isolates

To further characterize Pathfinder cell isolates, protein and mRNA profiles of various Pathfinder cells were studied. Protein and mRNA profiles may facilitate methods of identifying Pathfinder cells and potentially shed light on the mechanism of Pathfinder cell stimulation of repair.

Fluorescent antibody staining was performed on a panel of protein markers. Results are presented on Table 1. Markers were noted as positive if in the opinion of an experienced observer the fluorescent signal for the marker is significantly higher in the cells of interest than the background level of fluorescence for the experimental conditions used. Some of the proteins studied are typically found in stem cells, e.g., Oct3/4, Nanog, c-myc, which were found to be expressed in both human PDPCs and kDPCs. Sox2, another stem cell marker, was detectable in hPDPCs but not in hKPDCs. The overall protein expression patterns of hPDPCs and hkDPCs were consistent with that of an “unspecialized” cell type.

TABLE 3
Expression of proteins in hPatfinders (I removed tissue
source as we believe our cells are not tissue specific
and as determined by fluorescent antibody staining

| | | human PDPCs | | | | |
|---|---|---|---|---|---|---|
| | | + | | − | | |
| | | | | Proteins expressed in | | |
| | | Proteins expressed | | hkDPCs but not in hPDPCs | | |
| human | + | CD24* | Nanog | GFAP- | | |
| kDPCs | | c-myc | Oct4 | | | |
| | | HLA Class | Integrin | | | |
| | | 1 ABC | α2 + β1 | | | |
| | | ICAM3 | Ngn3 | | | |
| | | Nestin | CD130 | | | |
| | | Proteins expressed in | | Proteins expressed in | | |
| | | hPDPCs but not in hkDPCs | | neither hPDPCs nor hkDPCs | | |
| | − | Sox2 | | CD34 | CXCR2 | CD90 |
| | | CXCR4 | | CD105 | CD44 | ICAM1 |
| | | | | VCAM 1 | CD73 | NCAM |

*CD24 appears to be expressed above background only in small round cells in these cultures.

mRNA samples were prepared from human kidney-derived Pathfinder cells (hKDPCs) and lymph-node derived Pathfinder cells (hLNDPCs). mRNA expression profiles were determined for two large sets of genes by reverse-transcription-PCR(RT-PR) using ABI gene cards for a) genes known to be involved in stem cell pluripotency and b) genes known to be involved in cell cycle control. Tables 4 and 5 summarize results from these mRNA expression profiling experiments.

TABLE 4
mRNA expression results for genes known
to be involved in stem cell pluripotency

| | |
|---|---|
| Pluripotency genes | Pluripotency genes |
| expressed in | NOT expressed in |
| Pathinder cells | both Patfinder cells |

| | | | |
|---|---|---|---|
| RAF1 | ACTB | FOXA2 | LAMA1 |
| MYOD1 | ACTC | FOXD3 | LEFTB |
| SST | AFP | GABRB3 | LIFR |
| PAX6 | BRIZ | GAL | LIN28^{#} |
| ISL1 | CD34 | GAPDH | MYF5 |
| FN1 | CD9 | GATA4* | NANOG |
| LAMB1 | CDH5 | GATA6 | NES |
| IL6ST | CDX2 | GBX2 | NEUROD1 |
| | CGB | GCG | NODAL |
| | COL1A1 | GCM1 | NOG |
| | COL2A1 | GFAP | NPPA |
| | COMMD3 | GFAP | NR6A1 |
| | CRABP2 | GRB7 | NR5A2 |
| | CTNNB1 | HBB | OLIG2 |
| | DDX4 | HBZ | PAX4* |
| | DES | HLXB9 | PECAM1 |
| | DNMT3B | IAPP | PODXL |
| | EBAF | IFITM1 | POU5F1 |
| | EEF1A1 | IFITM2 | PTEN |
| | EOMES | IMP2 | PTF1A |
| | FGF4 | INS | REST |
| | FGF5 | IPF1 | RUNX2 |
| | FLT1 | KIT | SERPINA1 |
| | FN1 | KRT1 | SFRP2 |

*Not yet validated
^{#}LIN28 has a role in embryo cell differentiation and is a negative regulator of the let-7 miRNA family. Absence of LIN28 thus correlates with high levels of expression of the let-7 mRNA family in Pathfinder cells.

TABLE 5
mRNA expression results for genes known
to be involved in cell cycle control

| Cell cycle genes expressed | | Cell cycle genes NOT expressed | | |
|---|---|---|---|---|
| in Pathfinder cells | | in both Pathfinder cells | | |
| ATR | CDKN2C/p18 | GAPDH | CDC2 | HDAC2 |
| CCNB2 | E2F3 | HPRT1 | CDC25A | HDAC3 |
| CCND1 | E2F4 | GUSB | CDK4 | HDAC5 |
| CCNE1 | HDAC4 | ATM | CDKN1B (p27) | HDAC6 |
| CDK2 | HDAC9 | CCNA1 | CDKN2A (p16) | HDAC7 |
| CDK6 | PP2CA | CCNA2 | CDKN2B (p15) | RB1 |
| CDKN1A/p21 | RAF1 | CCNB1 | CDKN2D (p19) | TGFB1 |
| CDK7 | TGFβ3 | CCND2 | E2F1 | TGFB2 |
| | | CCND3 | E2F2 | TP53 |
| | | CCNE2 | GSK3B | |
| | | CCNH | HDAC1 | |

The degree of concordance in cell cycle control gene expression between human KDPCs and human LNDPCs is extraordinary, and strongly suggests that they are identical or closely related cell types. It is noteworthy that the cells do not express CDKN2A, which is typically required for senescence, indicating that our cell growth conditions and techniques are good. CDKN1A expression is observed, indicating that the cells can still undergo replicative senescence. Such an observation is desirable because it indicates that the cells are not transformed (i.e., not cancerous). Furthermore, the chromatin structure of the cells appears to be open (in keeping with a non-specialized status), as no expression was detected for a number of key histone deacetylase molecules. Only two HDACs, HDAC 4 and HDAC 9, were found to be expressed.

### Example 9

### Characterization of microRNA Expression in Pathfinder Cell Isolates

To further characterize and elucidate the mechanism of Pathfinder cell action, microRNA expression in hPDPCs, hKPDCs, and hLNDPCs were compared by RT-PCR using commercially available miRNA test cards. The resulting data show strong conservation of the miRNA expression profile of Pathfinder cell isolates from three different human tissues. (See Table 6.) Seventy-nine percent of the miRNAs with detectable levels of expression in the RNA preparations from these cell isolates were common to two or more of the isolates, while 64% were common to all three.

Further analyses are directed to determining if the observed differences are meaningful. Many of these miRNAs are present in very small amounts, which can lead to apparent differences between experiments and preparations. Many of the observed differences are due to a somewhat different miRNA expression profile of hPDPCs specifically. Without wishing to be bound by theory, these differences may relate to differences in timing of isolation and in adaptation of preparations to culture conditions.

TABLE 6
MicroRNA expression profiles of hPDPCs, hKDPCs, and hLNDPCs*

| Expression Pattern | miRNAs |
|---|---|
| Expressed in hPDPCs, | Let-7a, 7b, 7c, 7d, 7e, 7f, 7g |
| hKDPCs, and | miR-9, miR-10a, miR-10b, miR-15a, miR-15b, miR-16, miR-17, miR- |
| hLNDPCs (159) | 18a, miR-19a, miR-19b, miR-20a, miR-20b, miR-21, miR-22, miR- |
| | 23b, miR-24, miR-25, miR-26a, miR-26b, miR-27a, miR-27b, miR-28- |
| | 3p, miR-28-5p, miR-29a, miR-29c, miR-30a, miR-30b, miR-30c, miR- |
| | 30d, miR-30e, miR-32, miR-34a, miR-34c, miR-92a, miR-93, miR-98, |
| | miR-99a, miR-99b, miR-100, miR-101, miR-103, miR-106a, miR- |
| | 106b, miR-107, miR-125a-3p, miR-125a-5p, miR-125b, miR-126, |
| | miR-128, miR-130a, miR-130b, miR-132, miR-133a, miR-138, miR- |
| | 139-5p, miR-140-3p, miR-140-5p, miR-143, miR-145, miR-146a, miR- |
| | 146b-5p, miR-148b, miR-149, miR-150, miR-151-3p, miR-152, miR- |
| | 155, miR-181a, miR-181c, miR-184, miR-185, miR-186, miR-188-5p, |
| | miR-190, miR-191, miR-192, miR-193a-3p, miR-193a-5p, miR-193b, |
| | miR-194, miR-195, miR-197, miR-199a-3p, miR-199a-5p, miR-199b- |
| | 5p, miR-200b, miR-200c, miR-206, miR-210, miR-212, miR-214, |
| | miR-218, miR-221, miR-222, miR-223, miR-224, miR-296-5p, miR- |
| | 301a, miR-301b, miR-320, miR-323-3p, miR-324-3p, miR-324-5p, |
| | miR-328, miR-331-3p, miR-331-5p, miR-339-3p, miR-339-5p, miR- |
| | 361-5p, miR-365, miR-367, miR-374b, miR-378, miR-140, miR-422a, |
| | miR-423-5p, miR-425, miR-449b, miR-450a, miR-452, miR-483-5p, |
| | miR-484, miR-485-3p, miR-489, miR-494, miR-497, miR-509-3p, |
| | miR-516a-3p, miR-532-3p, miR-532-5p, miR-542-3p, miR-550, miR- |
| | 564, miR-572, miR-574-3p, miR-596, miR-610, miR-629, miR-639, |
| | miR-652, miR-661, miR-671-3p, miR-708, miR-744, miR-760, miR- |
| | 770-5p, miR-801, miR-875-5p, miR-877, miR-923, miR-939 |
| Expressed in hKPDCs | miR-7, miR-135b, miR-187, miR142-3p, miR-142-5p, miR-147b, miR- |
| and hLNDPCs but not | 18b, miR-196b, miR-204, miR-216a, miR-296-3p, miR-449a, miR- |
| in hPDPCs (18) | 502-5p, miR-509-5p, moR-582-5p, miR-615-3p, miR-636, miR-872 |
| Expressed in hPDPCs | miR-200a, miR-361-3p, miR-374p, miR-374a, miR-455 |
| and hLNDPCs but not | |
| in hKPDCs (4) | |
| Expressed in hPDPCs | miR-129-3p, miR-133b, miR-190b, miR-342-3p, miR-513-3p, miR- |
| and hKDPCs but not | 571, miR-592. miR-638, miR-657, miR-659, miR-766 |
| in hLNDPCs (11) | |
| Expressed in hKDPCs | miR-216b, miR-363, miR-505, miR-646 |
| (4) | |
| Expressed in hPDPCs | miR-3, miR-122, miR-127, miR-134, miR-137, miR-148a, miR-299- |
| (51) | 5p, miR-302b. miR-337-5p, miR-340, miR-345, miR-362-5p, miR- |
| | 369-5p, miR-370, miR-376a, miR-376, miR-379, miR-409-3p, miR- |
| | 411, miR-424, miR-432, miR-433, miR-450b, miR-454, miR-455-3p, |
| | miR-486, miR-487b, miR-490-3p, miR-491-5p, miR-493, miR-495, |
| | miR-500, miR-501-5p, miR-502-3p, miR-503, miR-539, miR-542-5p, |
| | iR-543, miR-549, miR-565, miR-590-5p, miR-625, miR-628-5p, miR- |
| | 655, miR-660, miR-668, miR-758, miR-768-3p, miR-769, miR-886-3p, |
| | miR-886-5p |

“Star miRNA” forms are also present in all rat and human progenitor cells
*The data are based on RT-PCR and thus depend on RNA quality and amount used, and on primer sets used by the manufacturer.

### Example 10

### Analysis of Secreted Factors of Pathfinder Cell Isolates

Our data thus far suggest that Pathfinder cells stimulate repair of damaged tissue by acting on host cells, rather than by integrating with the damaged tissue and making up a significant part of it. To explore the mechanism of Pathfinder cell action on host cells, ELISAs (enzyme-1inked immunosorbent assays) for a number of secreted protein factors were carried out on supernatants from hKDPCs and hPDPCs that were grown for thirty days in the original standard medium (i.e., not depleted of bovine microvesicles). Preliminary data from these experiments suggest that IL6, IGF-1 (hKDPCs and hPDPCs), and VEGF (hPDPCs only) are produced by these cells, and that the level of secretion of these factors changes with time in culture. These preliminary data are consistent with a capability of PDPCs to influence their local micro environment.

Pathfinder cells could act to assist repair of tissue damage (e.g, acute tissue damage) by direct or indirect mechanisms, or a combination of both. Stimulation of tissue repair from a state of complete loss of beta cells in the pancreas (as described in International Patent Publication No. WO 2006/120476) and reduction of cell senescence in a kidney damage model (as shown in Example 1) argue for predominantly direct effects of these cells.

Without wishing to be bound by any particular theory, the inventors propose a working model by which Pathfinder cells are attracted by soluble proteins and other signals from damaged tissue and possibly from macrophages. According to this working model, cell surface changes, (e.g., in cell adhesion molecules) in cells near a damage site make the damaged region recognizable, and this effect may extend for some distance. Reception of damage signals by Pathfinder cells may induce changes within these cells to convert them into active repair mode, e.g., increased cell mobility; production of secreted factors for tissue remodeling and reprogramming, growth factors, and pro-angiogenic and anti-coagulant factors; and changes in cell adhesion molecules and cell surface receptor proteins. Pathfinder cells may act on target cells of the damaged tissue to simulate the repair response, which may include activation of dormant stem cells or progenitors or induction of transdifferentiation (or de- and then re-differentiation) of adult cell type(s). This action could be via direct cell interaction and membrane protein-protein contact, and/or by the action of secreted proteins or nucleic acids.

As discussed in co-pending provisional patent application U.S. Ser. No. 61/373,715, filed on Aug. 13, 2010, regulatory microRNAs may be delivered within secreted microvesicles from pathfinder cells.

### Example 11

### Exemplary Protocols for Differentiation of Pathfinder Cells

The present Example provides examples of protocols for differentiation of pathfinder cells. See International Patent Publication WO2009/136168 for further details on methods used and for results previously obtained with these protocols.

### Pancreatic Differentiation

For pancreatic differentiation, pathfinder cells are plated at 6600 cells/cm² cell density. After 24 hours maintenance media is removed and monolayers are washed thrice with HBSS. Cells are subsequently cultured in DMEM:F12 (Lonza) supplemented with IxITS, 1.25 μg/ml Amphotericin B, and 100 μ/ml Penicillin/Streptomycin (all Invitrogen, UK), Nicotinamide 10 mM (Sigma), KGF 10 ng/ml (Sigma) and 0.2% BSA (Sigma). Medium changes are performed thrice weekly.

### Hepatogenic Differentation

For hepatogenic differentiation cells are plated at 6600 cells/cm² in T75 and 6 well plates, and at 2500 cells/cm² in chamber slides (Nunc). At 24 hours, maintenance media is replaced. After washing thrice with HBSS, cells are incubated in DMEM:F12 (Lonza) supplemented with Fibroblast Growth Factor-4 10 ng/ml (Sigma), 1×ITS, 100 μ/ml Penicillin/Streptomycin (Invitrogen), and 0.2% Bovine serum albumin (Sigma). Medium changes are performed thrice weekly.

### Preparation for Analyses

If desired, analyses to determine characteristics of differentiated cells, including morphological analyses, RT-PCR analysis, and staining for cell-specific markers can be determined as described previously. To prepare cells for analyses, cells are harvested for RNA extraction from undifferentiated pathfinder cells at day 0 and at day 28 for pancreatic differentiation and at Day 0, 7, 14, 21 and 28 for hepatic differentiation. Cells undergoing hepatic differentiation in a chamber slide are washed twice with PBS and fixed with 4% paraformaldehyde for 15 minutes at room temperature between days 10-14. Undifferentiated pathfinder cells also grown in chamber slides concurrently and were fixed at 90% confluency.

### Example 12

### Stimulation and Suppression of Cytokine Production by Pancreas-Derived Pathfinder Cell (PDPC) in a Mixed Lymphocyte Reaction (MLR)

The present Example demonstrates that pancreas-derived pathfinder cells (PDPCs) can stimulate and/or suppress cytokine response in a mixed lymphocyte reaction (MLR).

### Mixed Lymphocyte Reaction

On Day 1, flasks confluent with PDPCs with Mitomycin C (Sigma Aldrich, St. Louis, Mo., USA) in order to inhibit their proliferation. On Day 2, peripheral blood lymphocytes (PBLs) were harvested in Histopaque 1077 (Sigma Aldrich, St. Louis, Mo., USA) using standard procedures and optionally fractionated. PBLs were washed twice and counted to obtain 5×10⁶ cells/ml in proliferation medium (CMRL supplemented with FCS and beta-mercaptoethanol). PBLs were stored on ice until required. PDPCs were washed twice in HBSS, trypsinised and counted to obtain a top dose of 5×10⁴ cells/ml in proliferation medium. PDPCs were stored on ice until required. PDPCs were dispensed in an appropriate dose into a 96-well flat bottomed culture plate at 100 ul/well. Concanavalin A (Con A; Sigma Aldrich, St. Louis, Mo., USA) or phytohemagglutinin (PHA; Sigma Aldrich, St. Louis, Mo., USA) was added to the wells as appropriate. PBLs in an appropriate dose in 100 ul/well were added on top of PDPCs and incubated for 48 hrs. On Day 4, at least 150 ul culture supernatant was harvested from each well and transferred to a 96-well U-bottom plate and stored at −20° C. for cytokine ELISAs, which were performed using standard methods.

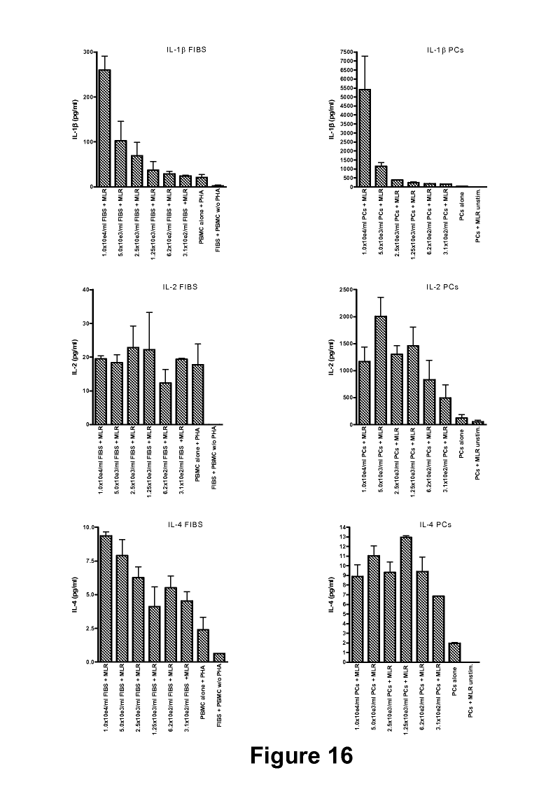

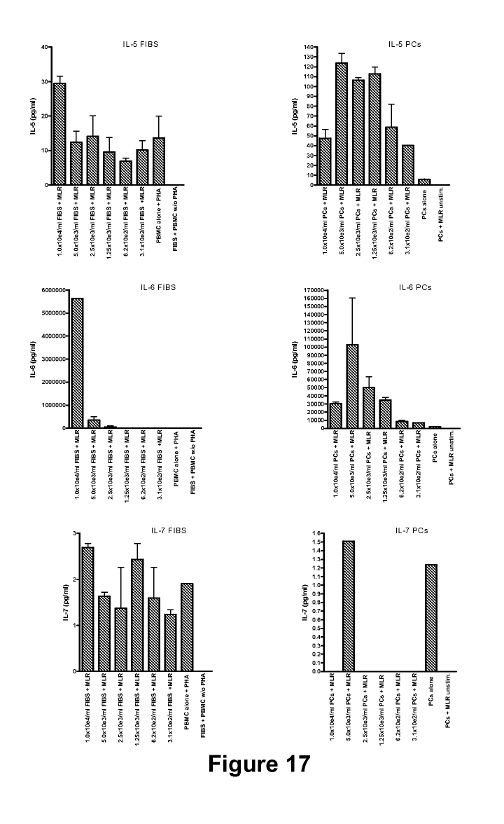

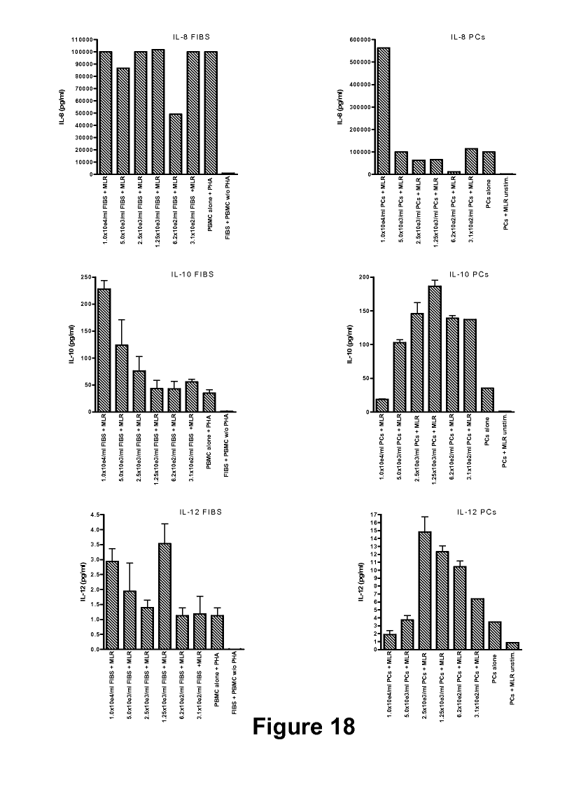

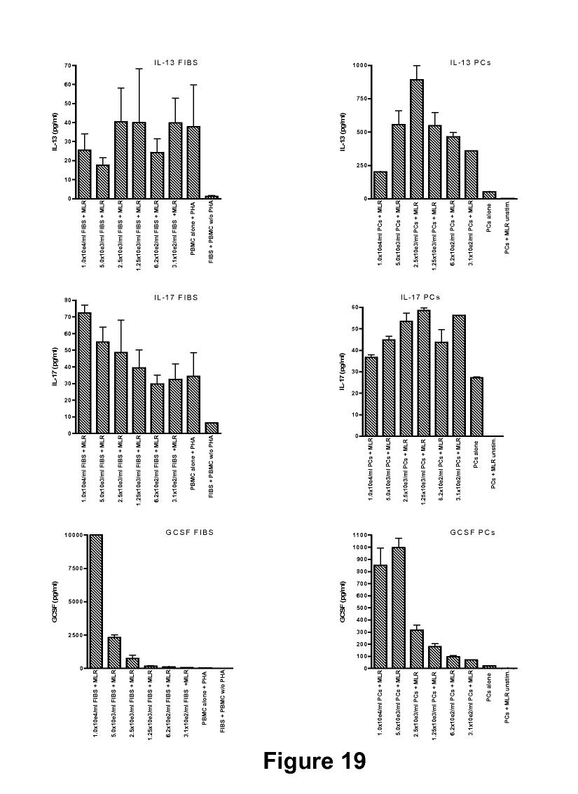

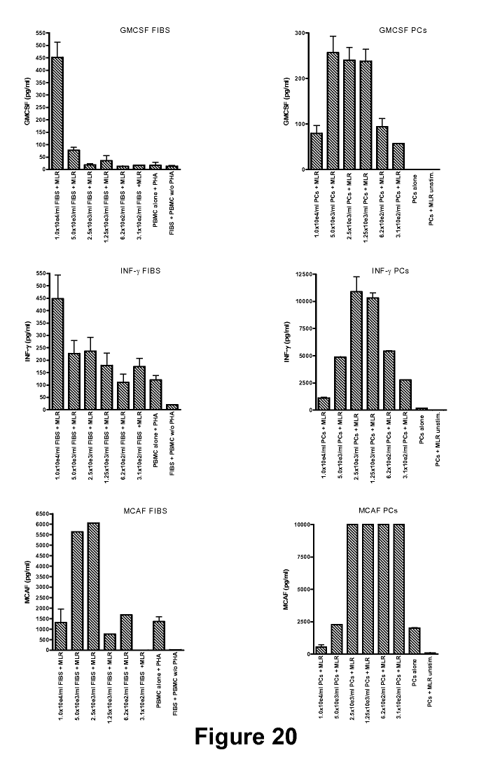

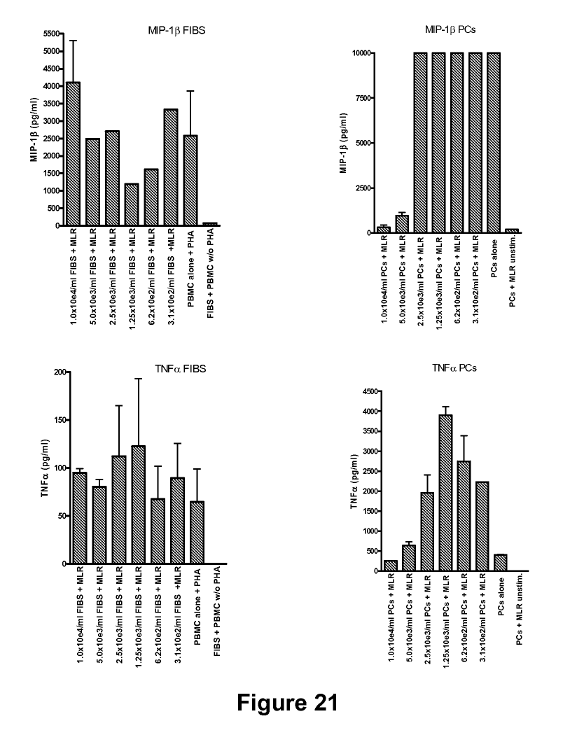

Mixed lymphocyte reactions using various amounts of PDPCs or control cells (FIBS) were performed and cytokine production was measured by ELISA. FIGS. 16-21 depict IL-1B, IL-2, IL-4, IL-5, IL-6, IL-7, IL-8, IL-10, IL-12, IL-13, IL-17, GCSF, GMCSF, IFN-g, MCAF, MIP-1B, and TNFa expression in response to incubation of MLR with PDPCs as compared to incubation of MLR with control cells. As can be seen, the PCs induce suppression of pro-inflammatory cytokines and anti-angiogenic factors. They appear to facilitate a suppression of T cell responses and provide a platform for the induction of immunological tolerance.

Taken together, these data suggest that PDPC induce an anti-inflammatory response, which may lead to immune tolerance. Without wishing to be bound by any particular theory, it is contemplated that PDPC induce expansion of cell types and/or expression of cytokines associated with anti-inflammatory response and induce decreased level and/or activity of cell types and/or cytokines associated with pro-inflammatory response.

### Example 13

### Stimulation and Suppression of Cytokine Production by Pancreas-Derived Pathfinder Cell (PDPC) in Mixed Lymphocyte Reaction (MLR) Fractions

The present Example demonstrates that pancreas-derived pathfinder cells (PDPCs) can stimulate and/or suppress cytokine response in a mixed lymphocyte reaction (MLR).

Sorting Peripheral Blood Leukocytes (PBLs) into T Cell Fractions

Blood was collected into EDTA and diluted 1:2 in MACS buffer. PBLs were harvested in Histopaque 1077 (Sigma Aldrich, St. Louis, Mo., USA) using standard procedures, as described in Example 12. T cells were isolated using Pan T Cell Isolation Kit (Miltenyi Biotec GmbH, Germany). Briefly, T cells pass through into the eluted fraction, leaving non-T cells bound to a magnetized column. The column was removed from the magnet and non-T cells were harvested (Fraction 1). T cells from the flow-through were then sorted for CD4 and CD25 using a CD4+CD25+ Regulatory T Cell Isolation Kit (Miltenyi Biotec GmbH, Germany), which yielded 3 further fractions: Fraction 2 (CD4− T cells), Fraction 3 (CD4+/CD25− T cells), and Fraction 4 (CD4+/CD25+ T cells).

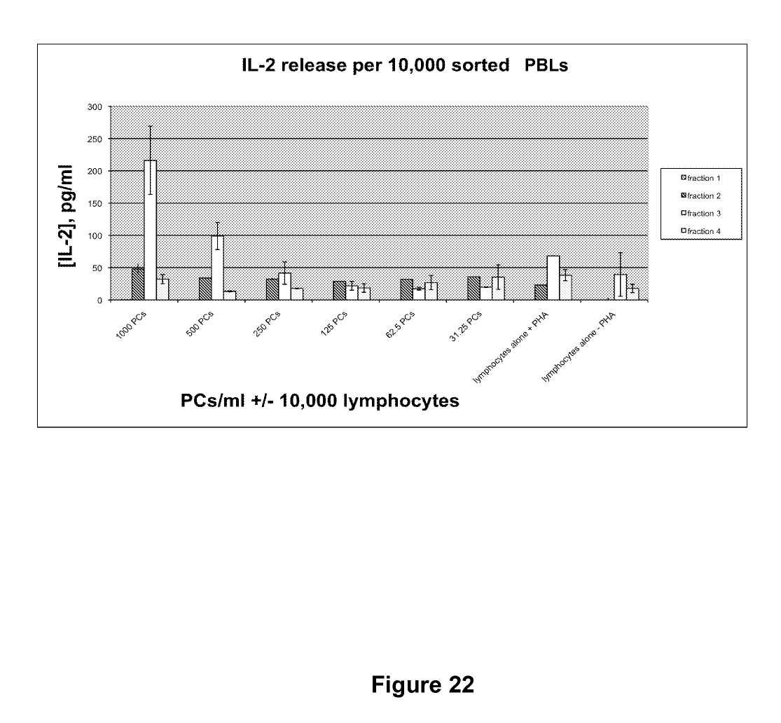

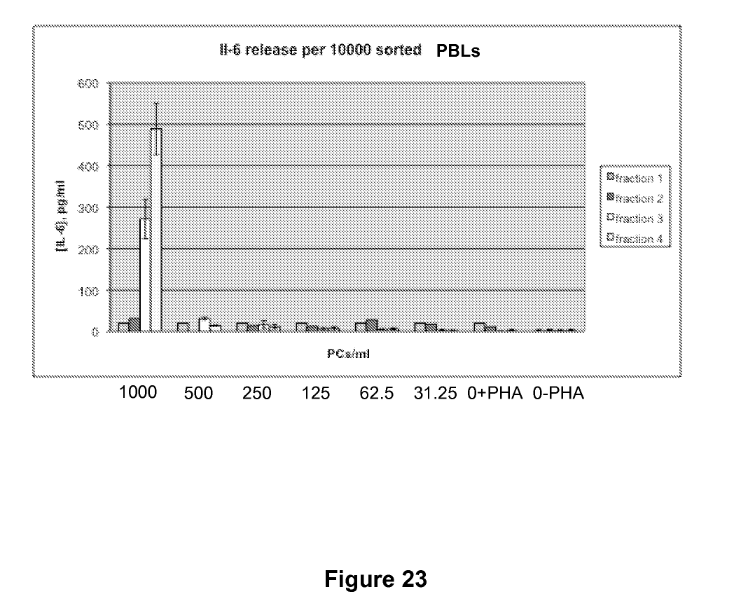

Mixed lymphocyte reactions using various amounts of PDPCs (e.g., 0-1,000 PCs/ml) were performed as described in Example 12 and cytokine production was measured by ELISA by standard methods. FIGS. 22 and 23 depict IL-2 and IL-6 release by each of Fractions 1-4.

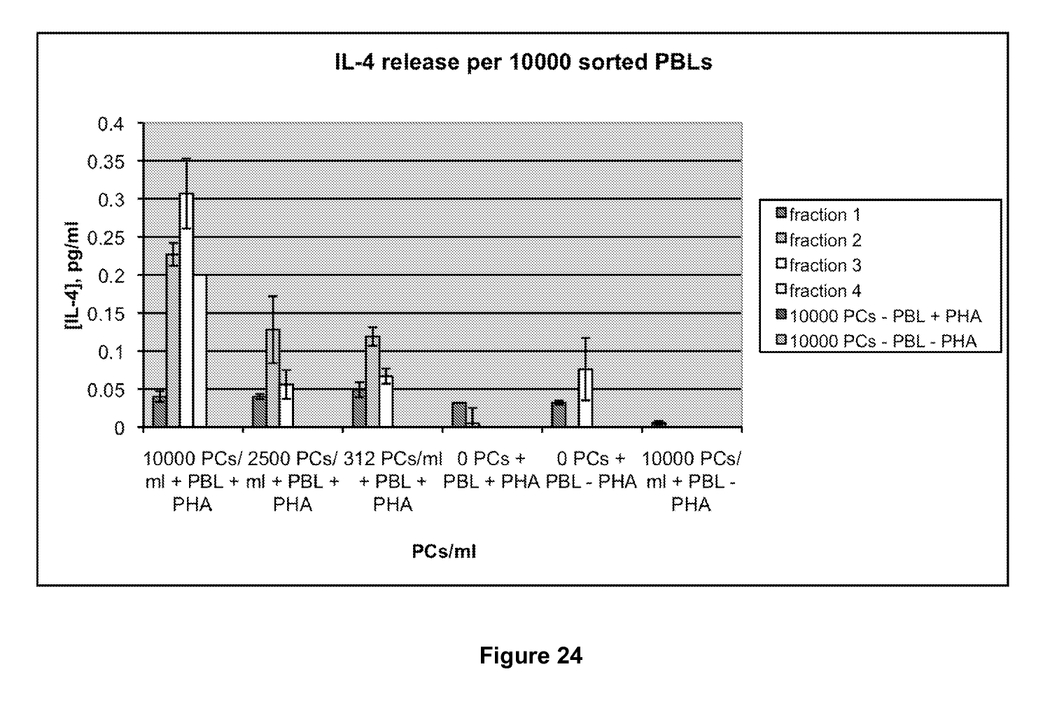

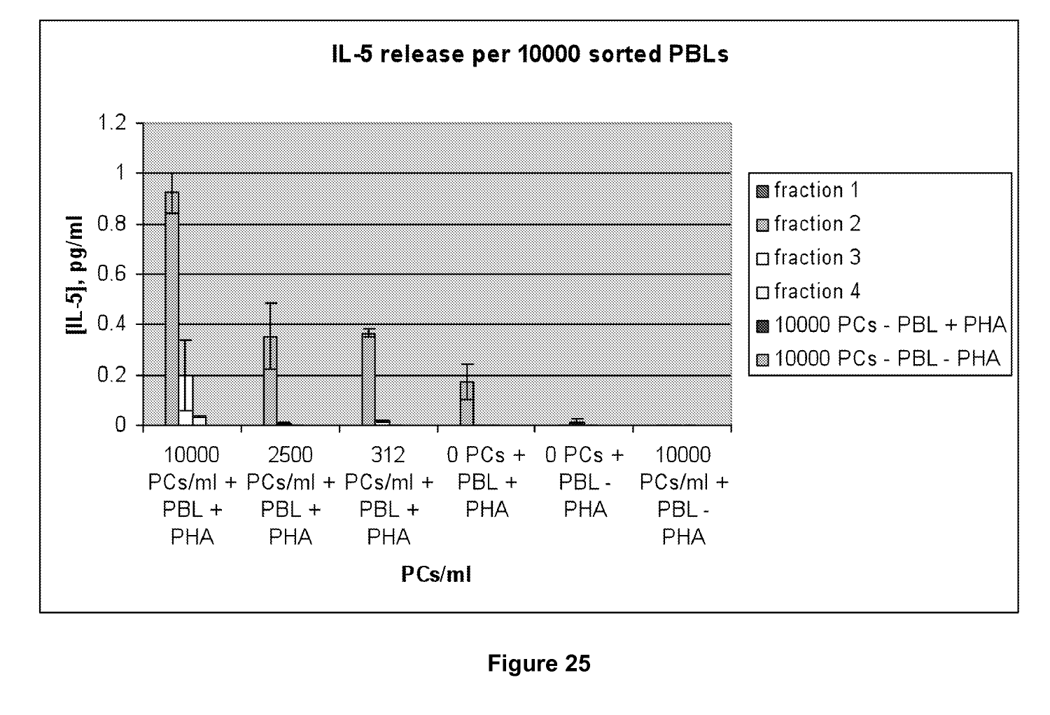

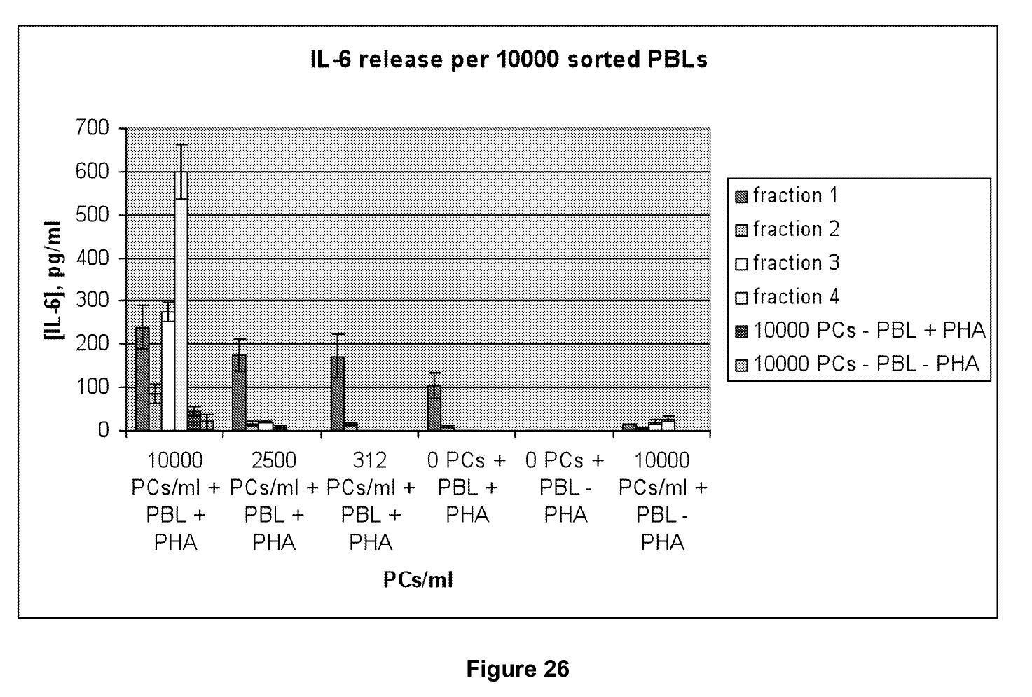

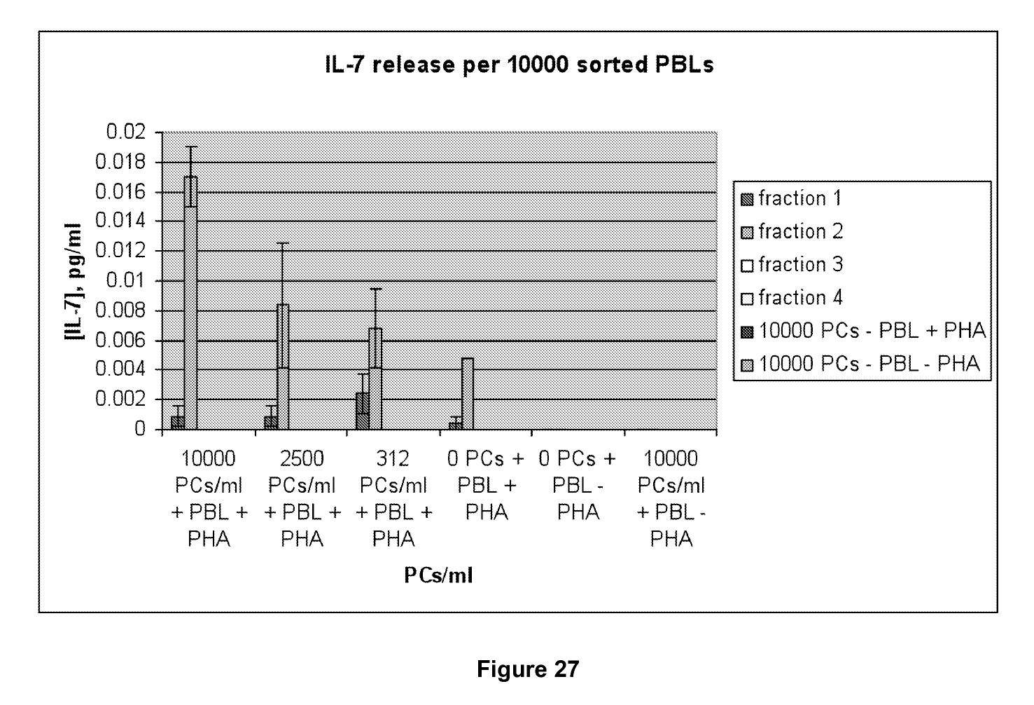

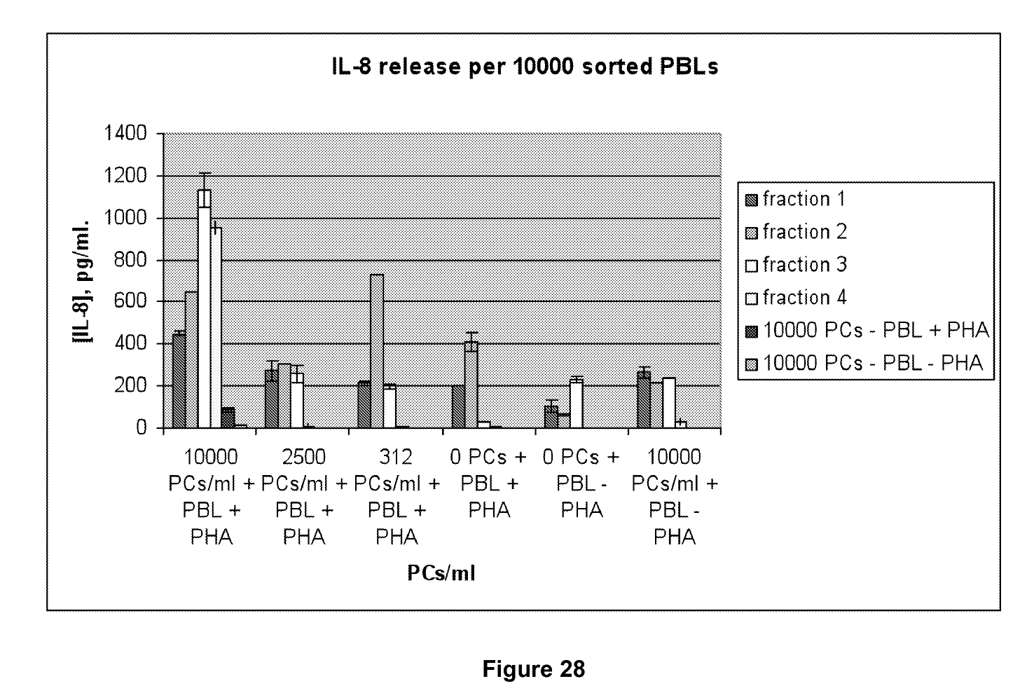

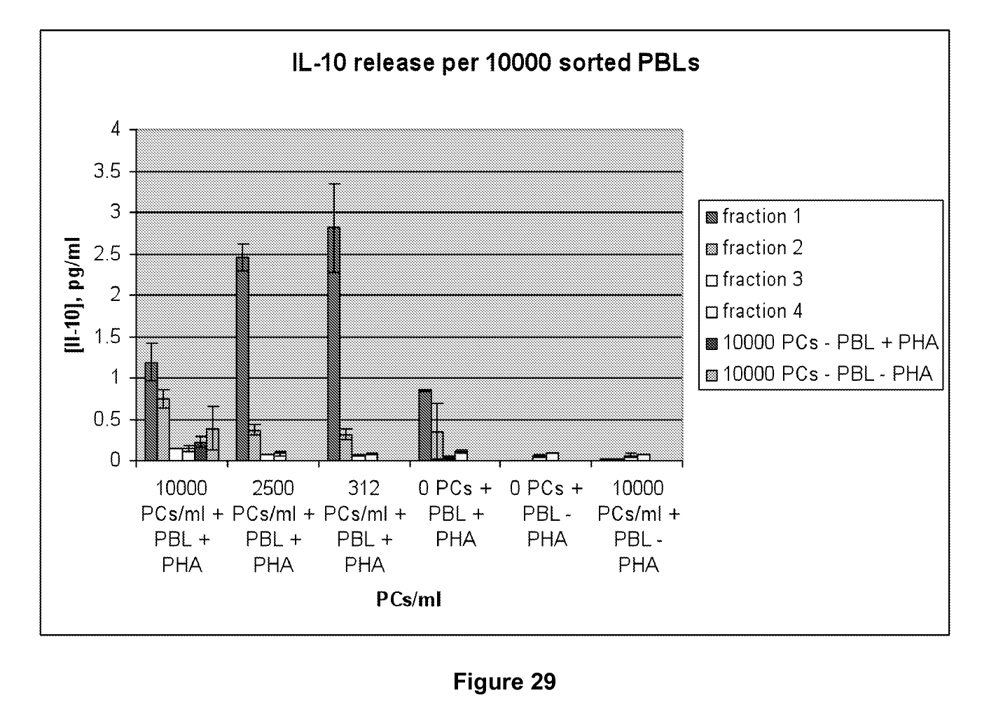

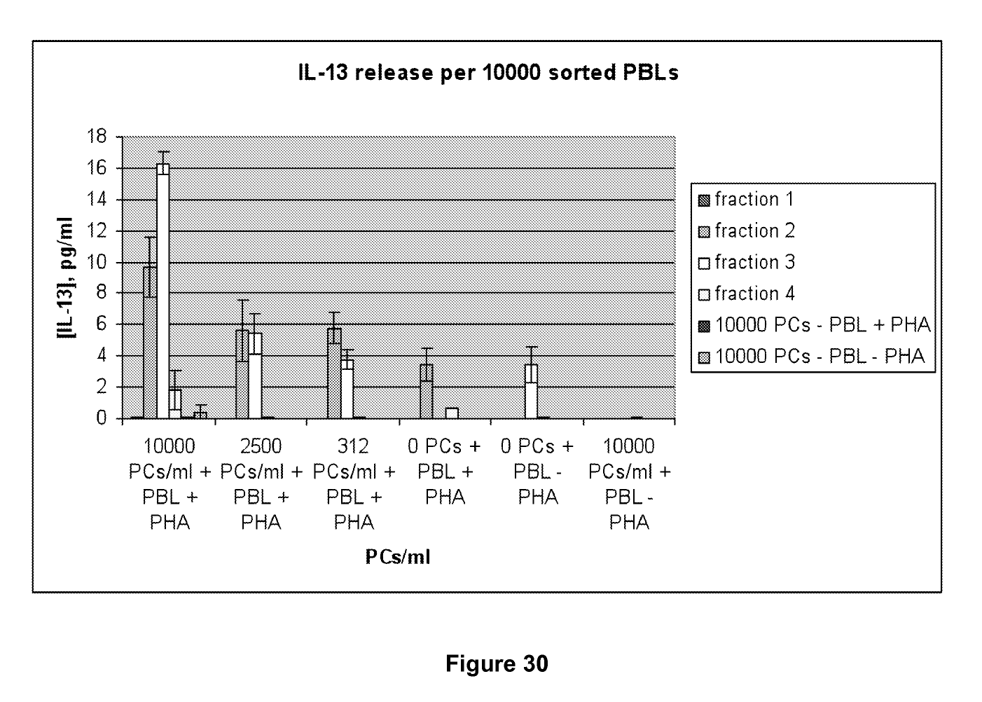

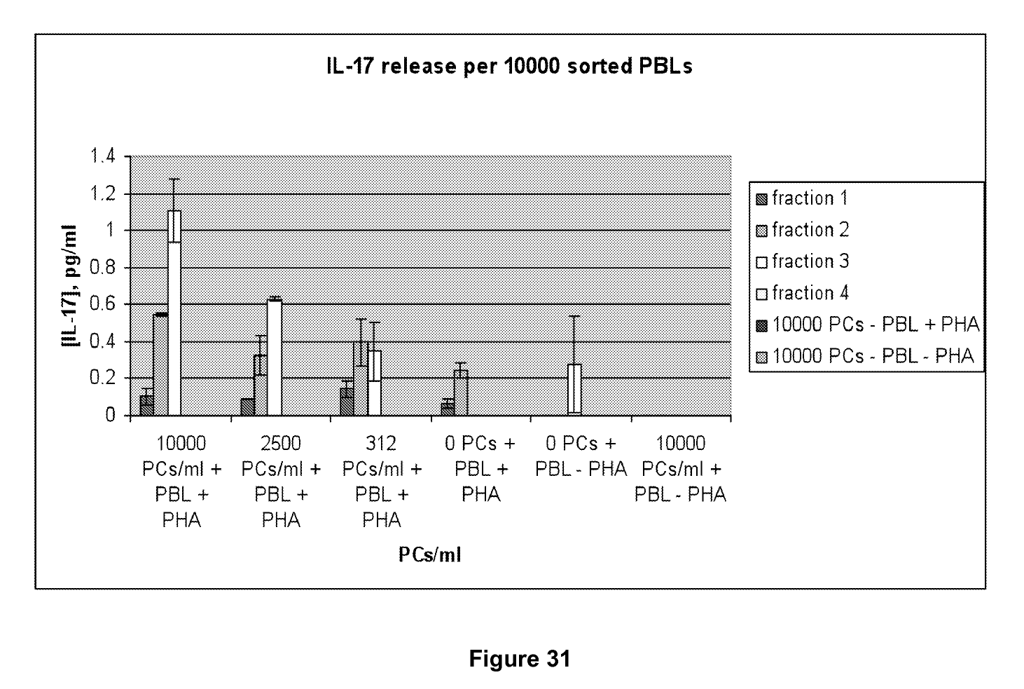

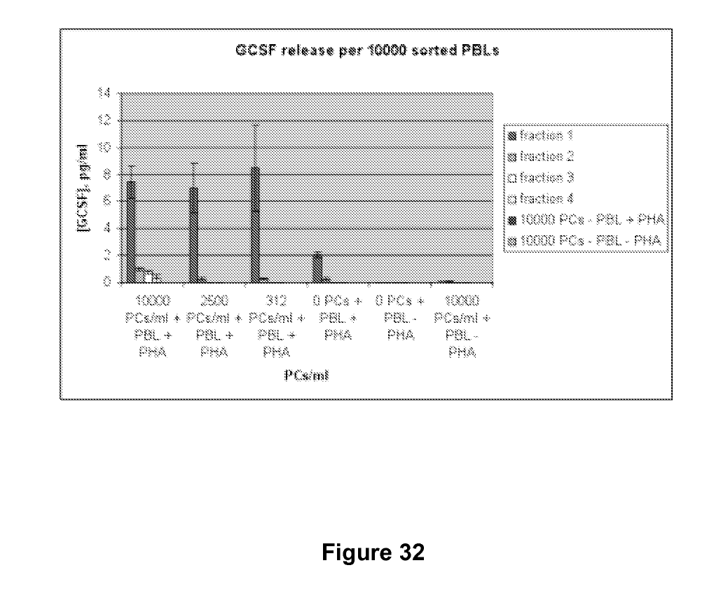

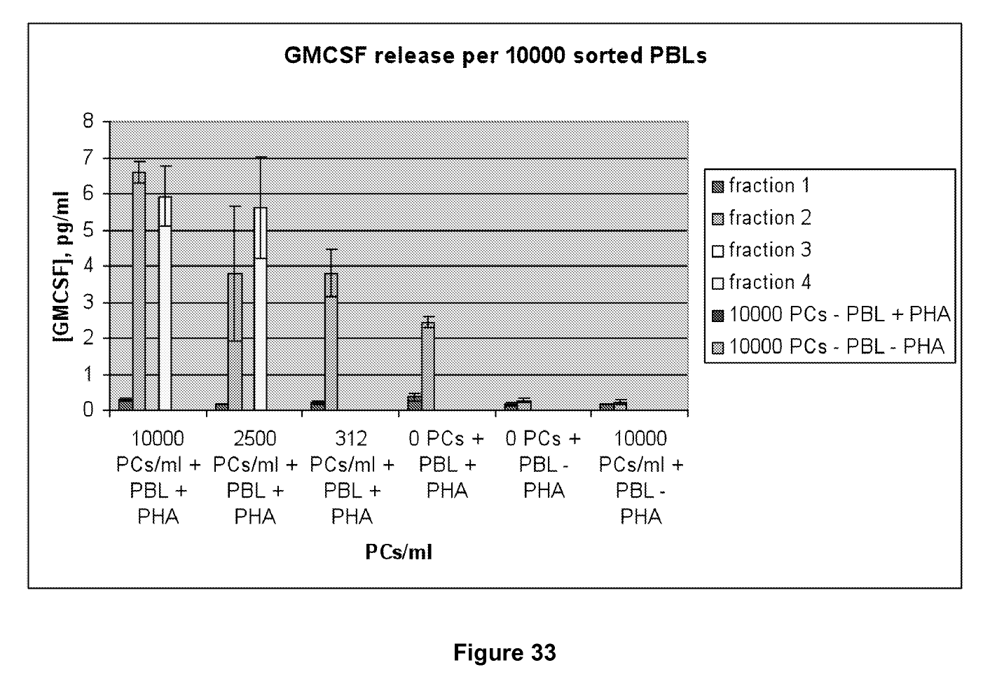

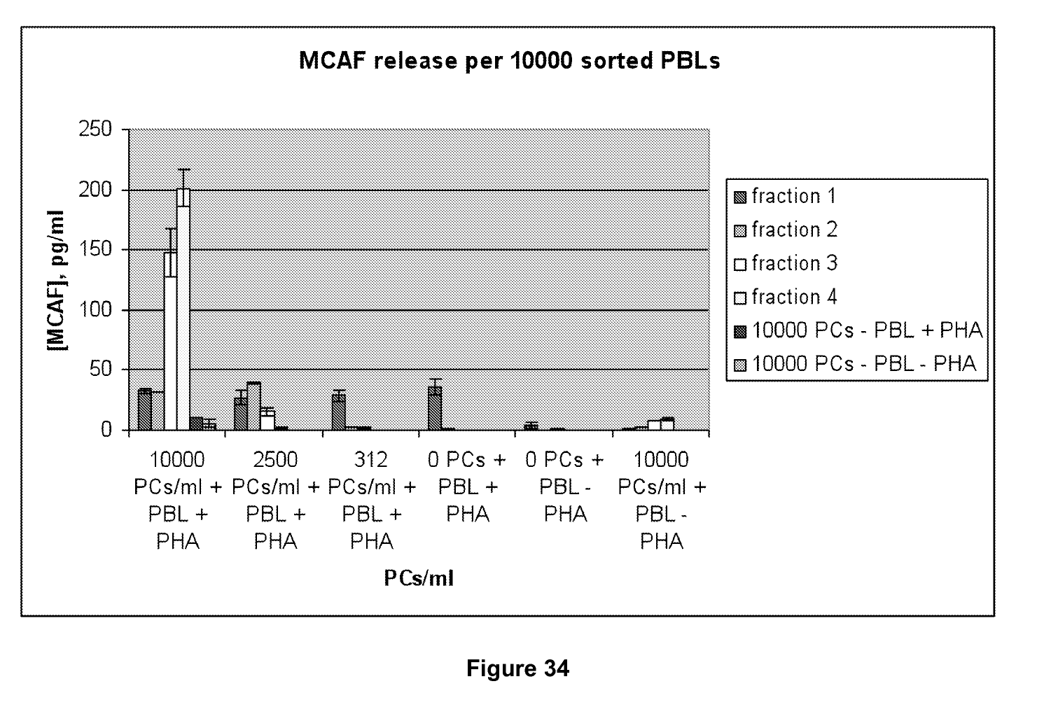

In another experiment, mixed lymphocyte reactions using various amounts of PDPCs (e.g., 0-10,000 PCs/ml) were performed as described in Example 12 and cytokine production was measured by ELISA by standard methods. FIGS. 24-35 depict IL-4, IL-5, IL-6, IL-7, IL-8, IL-10, IL-13, and IL-17, GCSF, GMCSF, and MCAF release by each of Fractions 1-4. It will be appreciated that differences in scale among MLR experiments may be due to different responses to PBLs in the reaction originating from different individuals.

Taken together, these data are consistent with a scenario of expansion of T regulatory cells and suppression of T cell responses, which may lead to induction of tolerance. Although IL-6 responses are complex, the data are consistent with induction of immunological tolerance and anergy.

### Example 14

### Stimulation and Suppression of Cytokine Production by Pancreas-Derived Pathfinder Cells (PDPC) and Mesenchymal Stem Cells (MSCs) in Mixed Lymphocyte Reaction (MLR)

The present Example demonstrates a comparison of cytokine and/or chemokine responses induced by pancreas-derived pathfinder cells (PDPCs) as compared to mesenchymal stem cells (MSCs) in a mixed lymphocyte reaction (MLR). Fractionation of PBLs was performed as described in Example 13. MLRs were performed as described in Example 12 by addition of PDPCs, MSC's or control, and cytokine and/or chemokine production was measured by ELISA by standard methods. Table 7 shows whether particular cytokines and/or chemokines are increased or decreased in response to PDPCs or MSCs as compared to a control in a mixed lymphocyte reaction. As can be seen, PDPCs induce a different cytokine profile than MSCs in the MLR (e.g., see IL-6, IL-4, IL-10 responses, among others).

TABLE 7
Cytokine/Chemokine profile induced by PDPCs
or MSCs in a mixed lymphocyte reaction (MLR)

| Cytokine/ | | | |
|---|---|---|---|
| Chemokine | PDPCs | MSC | Comment |
| IDO | | [symbol] | |
| Sol HLAg | | [symbol] | |
| IL-6 | [symbol] | [symbol] | |
| IL-4 | [symbol] | [symbol] | TH2 increased |
| | | | Il4 secretion |
| IL-10 | [symbol] | [symbol] | MSC Increased |
| | | | DC2s secretion |
| TGF beta | [symbol] | [symbol] | |
| TNFalpha | [symbol] | Stimulate decreased | |
| | | secretion by DC1s | |
| IL-12 | [symbol] | | |
| IFN gamma | [symbol] in MLR | [symbol] | MSC decreased |
| | Up in fraction 2 | | IFNg by TH1 |
| | CD4- T cells | | cells and NK |
| | | | cells |
| MCAF | [symbol] | | |
| MIP1 | [symbol] | | |
| GSCF | [symbol] | | |
| IL-1 beta | [symbol] In Fraction 1 | | |
| | (Non T cells) and | | |
| | Fraction 2 (CD4− | | |
| | Tcells) only | | |
| IL-5 | [symbol] | | |
| IL-8 | [symbol] | | |
| IL-17 | | | |
| IL-13 | [symbol] | | |
| GMCSF | [symbol] | | |

Without wishing to be bound by any particular theory, it is contemplated that PCs induce an increase in cytokines and/or chemokines that are typically associated with anti-inflammatory (e.g., tolerogenic) responses (e.g., IL-1β, GSCF, and IL-8), and induce a decrease in cytokines and/or chemokines that are typically associated with pro-inflammatory responses (e.g., IL-4, IL-5, IL-6, IL-10, IL-12, IL-13, IL-17, GMCSF, TGF-β, TNF-α, IFN-γ, MCAF, MIP1). These data show that IL-5, IL-7, and IL-13 responses may be mediated by a CD 4− T cell fraction containing gamma delta T cells. IL-13 is also mediated by CD4+25− T cells. The IL-10 response is principally via non-T cells, as is GCSF. GMCSF is principally released by regulatory T cells (T regs), while MCAF release is principally via T regs cells and CD4+CD25+ cells.

### Example 15

### RNA Expression Profiling of Pathfinder Cells (PCs) Cultured Under Serum Starvation Conditions

In the present Example, RNA from rat or human PDPC cells grown under serum starvation conditions were prepared using standard procedures. RNA samples were reverse-transcribed (RT) and amplified in a quantitative PCR assay in order to analyze expression of miRNAs.

### Materials and Methods

RNA extraction. RNA from cells was extracted using TRI Reagent (Sigma), with the following modifications to the manufacturer's protocol. After addition of ⅕th volume chloroform to the TRI Reagent, samples were spun at 6° C. for 15 minutes at 16,000×g. Aqueous phases were then subject to an extraction by phenol:choloform:isoamyl alcohol (pH 6.6; Ambion) at 10° C. for 10 minutes at 16,000×g. Aqueous phases were precipitated for a maximum of 2 hours at −20° C. After centrifugation at 6° C. for 30 minutes at 16,000×g, the resultant RNA was washed in 95% ice-cold ethanol. The RNA was then resuspended in DEPC-water and quantified using a NanoDrop 1000 spectrophotometer.

miRNA analysis. RNA from cells was analysed for expression of microRNAs (miRNAs) using Applied Biosystem's Taqman Low Density Arrays (TLDA) cards. For rat PDPCs, Taqman Rodent MicroRNA Arrays A and B were used in combination with MegaPlex RT Rodent Pool A and Pool B primers.

### Results

miRNAs in PCs were identified. Table 8 depicts results from analysis of miRNAs from rat PDPC RNA preparations.

TABLE 8
Exemplary miRNA sequences in serum starved rat PCs

| | | Alternative |
|---|---|---|
| miRNA | Exemplary Sequence(s)(5′ to 3′) | Description |
| mmu-miR-101a-4395364 | UACAGUACUGUGAUAACUGAAG (SEQ ID | MIMAT0000133 |
| | NO: 320) | |
| mmu-miR-10a-4373153 | UACCCUGUAGAUCCGAAUUUGUG (SEQ ID | MIMAT0000648 |
| | NO: 321) | |
| mmu-miR-10b-4395329 | UACCCUGUAGAACCGAAUUUGUG (SEQ ID | MIMAT0000208 |
| | NO: 322) | |
| mmu-miR-125a-3p-4395310 | ACAGGUGAGGUUCUUGGGAGCC (SEQ ID | MIMAT0004528 |
| | NO: 323) | |
| mmu-miR-125a-5p-4395309 | UCCCUGAGACCCUUUAACCUGUGA | MIMAT0000135 |
| | (SEQ ID NO: 324) | |
| mmu-miR-125b-3p-4395489 | ACGGGUUAGGCUCUUGGGAGCU (SEQ ID | MIMAT0004669 |
| | NO: 325) | |
| mmu-miR-128a-4395327 | UCACAGUCAACCGGUCUCUUU | MIMAT0000424 |
| | (SEQ ID NO: 326) | |
| mmu-miR-129-3p-4373297 | AAGCCCUUACCCCAAAAAGCAU (SEQ ID | MIMAT0000544 |
| | NO: 327) | |
| mmu-miR-138-4395395 | AGCUGGUGUUGUGAAUCAGGCCG (SEQ ID | MIMAT0000150 |
| | NO: 328) | |
| mmu-miR-142-3p-4373136 | UGUAGUGUUUCCUACUUUAUGGA (SEQ ID | MIMAT0000155 |
| | NO: 329) | |
| mmu-miR-142-5p-4395359 | CAUAAAGUAGAAAGCACUACU (SEQ ID | MIMAT0000154 |
| | NO: 330) | |
| mmu-miR-143-4395360 | UGAGAUGAAGCACUGUAGCUC (SEQ ID | MIMAT0000247 |
| | NO: 341) | |
| mmu-miR-146a-4373132 | UGAGAACUGAAUUCCAUGGGUU (SEQ ID | MIMAT0000158 |
| | NO: 342) | |
| mmu-miR-147-4395373 | GUGUGCGGAAAUGCUUCUGCUA (SEQ ID | MIMAT0004857 |
| | NO: 343) | |
| mmu-miR-148a-4373130 | UCAGUGCACUACAGAACUUUGU (SEQ ID | MIMAT0000516 |
| | NO: 344) | |
| mmu-miR-148b-4373129 | UCAGUGCAUCACAGAACUUUGU (SEQ ID | MIMAT0000580 |
| | NO: 345) | |
| mmu-miR-151-3p-4373304 | CUAGACUGAGGCUCCUUGAGG (SEQ ID | MIMAT0000161 |
| | NO: 346) | |
| mmu-miR-182-4395729 | UUUGGCAAUGGUAGAACUCACACCG (SEQ | MIMAT0000211 |
| | ID NO: 347) | |
| mmu-miR-187-4373307 | UCGUGUCUUGUGUUGCAGCCGG (SEQ ID | MIMAT0000216 |
| | NO: 348) | |
| mmu-miR-188-5p-4395431 | CAUCCCUUGCAUGGUGGAGGG (SEQ ID | MIMAT0000217 |
| | NO: 349) | |
| mmu-miR-18a-4395533 | UAAGGUGCAUCUAGUGCAGAUAG (SEQ ID | MIMAT0000528 |
| | NO: 350) | |
| mmu-miR-190-4373110 | UGAUAUGUUUGAUAUAUUAGGU (SEQ ID | MIMAT0000220 |
| | NO: 351) | |
| mmu-miR-196b-4395326 | UAGGUAGUUUCCUGUUGUUGGG (SEQ ID | MIMAT0001081 |
| | NO: 352) | |
| mmu-miR-197-4373102 | UUCACCACCUUCUCCACCCAGC | MIMAT0000227 |
| | (SEQ ID NO: 353) | |
| mmu-miR-199a-3p-4395415 | ACAGUAGUCUGCACAUUGGUUA (SEQ ID | MIMAT0000230 |
| | NO: 354) | |
| mmu-miR-200c-4395411 | UAAUACUGCCGGGUAAUGAUGGA (SEQ ID | MIMAT0000657 |
| | NO: 355) | |
| mmu-miR-204-4373094 | UUCCCUUUGUCAUCCUAUGCCU (SEQ ID | MIMAT0000237 |
| | NO: 356) | |
| mmu-miR-210-4373089 | CUGUGCGUGUGACAGCGGCUGA (SEQ ID | MIMAT0000658 |
| | NO: 357) | |
| mmu-miR-21-4373090 | UAGCUUAUCAGACUGAUGUUGA (SEQ ID | MIMAT0000530 |
| | NO: 358) | |
| mmu-miR-222-4395387 | AGCUACAUCUGGCUACUGGGU (SEQ ID | MIMAT0000670 |
| | NO: 359) | |
| mmu-miR-23a-4373074 | AUCACAUUGCCAGGGAUUUCC (SEQ ID | MIMAT0000532 |
| | NO: 360) | |
| mmu-miR-23b-4373073 | AUCACAUUGCCAGGGAUUACC (SEQ ID | MIMAT0000125 |
| | NO: 361) | |
| mmu-miR-26a-4395166 | UUCAAGUAAUCCAGGAUAGGCU (SEQ ID | MIMAT0000533 |
| | NO: 362) | |
| mmu-miR-29b-4373288 | UAGCACCAUUUGAAAUCAGUGUU (SEQ ID | MIMAT0000127 |
| | NO: 363) | |
| mmu-miR-29c-4395171 | UAGCACCAUUUGAAAUCGGUUA (SEQ ID | MIMAT0000536 |
| | NO: 364) | |
| mmu-miR-320-4395388 | AAAAGCUGGGUUGAGAGGGCGA (SEQ ID | MIMAT0000666 |
| | NO: 365) | |
| mmu-miR-322-4378107 | CAGCAGCAAUUCAUGUUUUGGA (SEQ ID | MIMAT0000548 |
| | NO: 366) | |
| mmu-miR-324-5p-4373052 | CGCAUCCCCUAGGGCAUUGGUGU (SEQ ID | MIMAT0000555 |
| | NO: 367) | |
| mmu-miR-331-5p-4395344 | CUAGGUAUGGUCCCAGGGAUCC (SEQ ID | MIMAT0004643 |
| | NO: 368) | |
| mmu-miR-335-3p-4395296 | UUUUUCAUUAUUGCUCCUGACC (SEQ ID | MIMAT0004704 |
| | NO: 369) | |
| mmu-miR-339-5p-4395368 | UCCCUGUCCUCCAGGAGCUCACG (SEQ ID | MIMAT0000584 |
| | NO: 370) | |
| mmu-miR-345-5p-4395658 | GCUGACCCCUAGUCCAGUGCUU (SEQ ID | MIMAT0000595 |
| | NO: 371) | |
| mmu-miR-350-4395660 | UUCACAAAGCCCAUACACUUUC (SEQ ID | MIMAT0000605 |
| | NO: 372) | |
| mmu-miR-351-4373345 | UCCCUGAGGAGCCCUUUGAGCCUG (SEQ | MIMAT0000609 |
| | ID NO: 373) | |
| mmu-miR-361-4373035 | UUAUCAGAAUCUCCAGGGGUAC (SEQ ID | MIMAT0000704 |
| | NO: 374) | |
| mmu-miR-362-3p-4395746 | AACACACCUGUUCAAGGAUUCA (SEQ ID | MIMAT0004684 |
| | NO: 375) | |
| mmu-miR-384-5p-4395732 | UGUAAACAAUUCCUAGGCAAUGU (SEQ ID | MIMAT0004745 |
| | NO: 376) | |
| mmu-miR-429-4373355 | UAAUACUGUCUGGUAAUGCCGU (SEQ ID | MIMAT0001537 |
| | NO: 377) | |
| mmu-miR-450a-5p-4395414 | UUUUGCGAUGUGUUCCUAAUAU (SEQ ID | MIMAT0001546 |
| | NO: 378) | |
| mmu-miR-494-4395476 | UGAAACAUACACGGGAAACCUC (SEQ ID | MIMAT0003182 |
| | NO: 379) | |
| mmu-miR-500-4395736 | AAUGCACCUGGGCAAGGGUUCA (SEQ ID | MIMAT0003507 |
| | NO: 380) | |
| mmu-miR-503-4395586 | UAGCAGCGGGAACAGUACUGCAG (SEQ ID | MIMAT0003188 |
| | NO: 381) | |
| mmu-miR-542-3p-4378101 | UGUGACAGAUUGAUAACUGAAA (SEQ ID | MIMAT0003172 |
| | NO: 382) | |
| mmu-miR-582-3p-4395697 | CCUGUUGAACAACUGAACCCAA (SEQ ID | MIMAT0005292 |
| | NO: 383) | |
| mmu-miR-582-5p-4395696 | UACAGUUGUUCAACCAGUUACU (SEQ ID | MIMAT0005291 |
| | NO: 384) | |
| mmu-miR-598-4395606 | UACGUCAUCGUCGUCAUCGUUA (SEQ ID | MIMAT0004942 |
| | NO: 385) | |
| mmu-miR-652-4395463 | AAUGGCGCCACUAGGGUUGUG (SEQ ID | MIMAT0003711 |
| | NO: 386) | |
| mmu-miR-667-4386769 | UGACACCUGCCACCCAGCCCAAG (SEQ ID | MIMAT0003734 |
| | NO: 387) | |
| mmu-miR-685-4386748 | UCAAUGGCUGAGGUGAGGCAC | MIMAT0003463 |
| | (SEQ ID NO: 388) | |
| mmu-miR-743b-5p-4395600 | UGUUCAGACUGGUGUCCAUCA (SEQ ID | MIMAT0004839 |
| | NO: 389) | |
| mmu-miR-744-4395435 | UGCGGGGCUAGGGCUAACAGCA (SEQ ID | MIMAT0004187 |
| | NO: 390) | |
| mmu-miR-883a-3p-4395591 | UAACUGCAACAGCUCUCAGUAU (SEQ ID | MIMAT0004849 |
| | NO: 391) | |
| mmu-miR-883b-3p-4395695 | UAACUGCAACAUCUCUCAGUAU (SEQ ID | MIMAT0004851 |
| | NO: 392) | |
| mmu-miR-98-4373009 | UGAGGUAGUAAGUUGUAUUGUU (SEQ ID | MIMAT0000545 |
| | NO: 393) | |
| rno-miR-190b-4395749 | UGAUAUGUUUGAUAUUAGGUU (SEQ ID | MIMAT0005302 |
| | NO: 394) | |
| rno-miR-207-4381096 | GCUUCUCCUGGCUCUCCUCCCUU (SEQ ID | MIMAT0003115 |
| | NO: 395) | |
| rno-miR-333-4381109 | GUGGUGUGCUAGUUACUUUU | |
| | (SEQ ID NO: 396) | |
| rno-miR-339-3p-4395760 | UGAGCGCCUCGACGACAGAGCCA (SEQ ID | MIMAT0004648 |
| | NO: 397) | |
| rno-miR-345-3p-4395762 | CCCUGAACUAGGGGUCUGGAGA (SEQ ID | MIMAT0004655 |
| | NO: 398) | |
| rno-miR-351-4395764 | UCCCUGAGGAGCCCUUUGAGCCUGA (SEQ | MIMAT0000608 |
| | ID NO: 399) | |
| rno-miR-466c-4395768 | UGUGAUGUGUGCAUGUACAUG (SEQ ID | MIMAT0005279 |
| | NO: 400) | |
| rno-miR-743b-4395769 | GAAAGACACCAUACUGAAUAGA (SEQ ID | MIMAT0005280 |
| | NO: 401) | |
| snoRNA202-4380914 | GCTGTACTGACTTGATGAAAGTACTTTTGA | |
| | ACCCTTTTCCATCTGATG | |
| | (SEQ ID NO: 402) | |
| mmu-let-7f*-4395528 | CUAUACAAUCUAUUGCCUUCCC (SEQ ID | MIMAT0004623 |
| | NO: 403) | |
| mmu-let-7g*-4395622 | ACUGUACAGGCCACUGCCUUGC (SEQ ID | MIMAT0004519 |
| | NO: 404) | |
| mmu-let-7i*-4395283 | CUGCGCAAGCUACUGCCUUGCU (SEQ ID | MIMAT0004520 |
| | NO: 405) | |
| mmu-miR-106b*-4395491 | CCGCACUGUGGGUACUUGCUGC (SEQ ID | MIMAT0004582 |
| | NO: 406) | |
| mmu-miR-10a*-4395399 | CAAAUUCGUAUCUAGGGGAAUA (SEQ ID | MIMAT0004659 |
| | NO: 407) | |
| mmu-miR-10b*-4395702 | CAGAUUCGAUUCUAGGGGAAUA (SEQ ID | MIMAT0004538 |
| | NO: 408) | |
| mmu-miR-130b*-4395590 | ACUCUUUCCCUGUUGCACUACU (SEQ ID | MIMAT0004583 |
| | NO: 409) | |
| mmu-miR-135a*-4395343 | UAUAGGGAUUGGAGCCGUGGCG (SEQ ID | MIMAT0004531 |
| | NO: 410) | |
| mmu-miR-149-4395366 | UCUGGCUCCGUGUCUUCACUCCC (SEQ ID | MIMAT0000159 |
| | NO: 411) | |
| mmu-miR-15b*-4395284 | CGAAUCAUUAUUUGCUGCUCUA (SEQ ID | MIMAT0004521 |
| | NO: 412) | |
| mmu-miR-16*-4395619 | CCAGUAUUGACUGUGCUGCUGA (SEQ ID | MIMAT0004625 |
| | NO: 413) | |
| mmu-miR-17*-4395673 | ACUGCAGUGAGGGCACUUGUAG (SEQ ID | MIMAT0000650 |
| | NO: 414) | |
| mmu-miR-18a*-4395620 | ACUGCCCUAAGUGCUCCUUCUG (SEQ ID | MIMAT0004626 |
| | NO: 415) | |
| mmu-miR-191*4395706 | GCUGCACUUGGAUUUCGUUCCC (SEQ ID | MIMAT0004542 |
| | NO: 416) | |
| mmu-miR-199b*-4373309 | CCCAGUGUUUAGACUACCUGUUC (SEQ ID | MIMAT0000672 |
| | NO: 417) | |
| mmu-miR-206-4373092 | UGGAAUGUAAGGAAGUGUGUGG (SEQ ID | MIMAT0000239 |
| | NO: 418) | |
| mmu-miR-214*-4395404 | UGCCUGUCUACACUUGCUGUGC (SEQ ID | MIMAT0004664 |
| | NO: 419) | |
| mmu-miR-218-1*-4395682 | AAACAUGGUUCCGUCAAGCACC (SEQ ID | MIMAT0004665 |
| | NO: 420) | |
| mmu-miR-24-1*-4378067 | GUGCCUACUGAGCUGAUAUCAGU (SEQ ID | MIMAT0000218 |
| | NO: 421) | |
| mmu-miR-26b*-4395555 | CCUGUUCUCCAUUACUUGGCUC (SEQ ID | MIMAT0004630 |
| | NO: 422) | |
| mmu-miR-291a-5p-4373322 | CAUCAAAGUGGAGGCCCUCUCU (SEQ ID | MIMAT0000367 |
| | NO: 423) | |
| mmu-miR-297a*-4395584 | UAUACAUACACACAUACCCAUA (SEQ ID | MIMAT0004864 |
| | NO: 424) | |
| mmu-miR-29a*-4395558 | ACUGAUUUCUUUUGGUGUUCAG (SEQ ID | MIMAT0004631 |
| | NO: 425) | |
| mmu-miR-29b*-4395627 | GCUGGUUUCAUAUGGUGGUUUA (SEQ ID | MIMAT0004523 |
| | NO: 426) | |
| mmu-miR-29c*-4381131 | UGACCGAUUUCUCCUGGUGUUC (SEQ ID | MIMAT0004632 |
| | NO: 427) | |
| mmu-miR-30a*-4373062 | CUUUCAGUCGGAUGUUUGCAGC (SEQ ID | MIMAT0000129 |
| | NO: 428) | |
| mmu-miR-30b*-4395628 | CUGGGAUGUGGAUGUUUACGUC (SEQ ID | MIMAT0004524 |
| | NO: 429) | |
| mmu-miR-30c-1*-4395219 | CUGGGAGAGGGUUGUUUACUCC (SEQ ID | MIMAT0004616 |
| | NO: 430) | |
| mmu-miR-30e*-4373057 | CUUUCAGUCGGAUGUUUACAGC (SEQ ID | MIMAT0000249 |
| | NO: 431) | |
| mmu-miR-322*-4395636 | AAACAUGAAGCGCUGCAACAC (SEQ ID | MIMAT0000549 |
| | NO: 432) | |
| mmu-miR-326-4373335 | CCUCUGGGCCCUUCCUCCAGU (SEQ ID | MIMAT0000559 |
| | NO: 433) | |
| mmu-miR-330*-4373337 | GCAAAGCACAGGGCCUGCAGAGA (SEQ ID | MIMAT0000569 |
| | NO: 331) | |
| mmu-miR-374-4381045 | AUAUAAUACAACCUGCUAAGUG (SEQ ID | MIMAT0003727 |
| | NO: 434) | |
| mmu-miR-378*-4373024 | CUCCUGACUCCAGGUCCUGUGU (SEQ ID | MIMAT0000742 |
| | NO: 435) | |
| mmu-miR-378-4395354 | ACUGGACUUGGAGUCAGAAGG (SEQ ID | MIMAT0003151 |
| | NO: 436) | |
| mmu-miR-425*-4373202 | AUCGGGAAUGUCGUGUCCGCC (SEQ ID | MIMAT0001342 |
| | NO: 437) | |
| mmu-miR-466d-3p-4395665 | UAUACAUACACGCACACAUAG (SEQ ID | MIMAT0004931 |
| | NO: 438) | |
| mmu-miR-467a*-4386757 | AUAUACAUACACACACCUACAC (SEQ ID | MIMAT0002108 |
| | NO: 439) | |
| mmu-miR-467b*-4381092 | AUAUACAUACACACACCAACAC (SEQ ID | MIMAT0003478 |
| | NO: 440) | |
| mmu-miR-503*-4395666 | GAGUAUUGUUUCCACUGCCUGG (SEQ ID | MIMAT0004790 |
| | NO: 441) | |
| mmu-miR-673-5p-4386772 | CUCACAGCUCUGGUCCUUGGAG (SEQ ID | MIMAT0003739 |
| | NO: 442) | |
| mmu-miR-674*-4386773 | CACAGCUCCCAUCUCAGAACAA (SEQ ID | MIMAT0003741 |
| | NO: 443) | |
| mmu-miR-678-4381076 | GUCUCGGUGCAAGGACUGGAGG (SEQ ID | MIMAT0003452 |
| | NO: 444) | |
| mmu-miR-692-4381088 | AUCUCUUUGAGCGCCUCACUC (SEQ ID | MIMAT0003471 |
| | NO: 445) | |
| mmu-miR-699-4381056 | AGGCAGUGCGACCUGGCUCG | MIMAT0003489 |
| | (SEQ ID NO: 446) | |
| mmu-miR-720-4381052 | AUCUCGCUGGGGCCUCCA (SEQ ID NO: 447) | MIMAT0003484 |
| mmu-miR-721-4381073 | CAGUGCAAUUAAAAGGGGGAA (SEQ ID | MIMAT0003515 |
| | NO: 448) | |
| mmu-miR-744*-4395436 | CUGUUGCCACUAACCUCAACCU (SEQ ID | MIMAT0004820 |
| | NO: 449) | |
| mmu-miR-760-4395439 | CGGCUCUGGGUCUGUGGGGA (SEQ ID | MIMAT0003898 |
| | NO: 450) | |
| mmu-miR-801-4395562 | GAUUGCUGUGCGUGCGGAAUCGAC | |
| | (SEQ ID NO: 451) | |
| mmu-miR-875-5p-4395314 | UAUACCUCAGUUUUAUCAGGUG (SEQ ID | MIMAT0004937 |
| | NO: 452) | |
| mmu-miR-877-4395402 | GUAGAGGAGAUGGCGCAGGG (SEQ ID | MIMAT0004861 |
| | NO: 453) | |
| mmu-miR-9*-4395342 | AUAAAGCUAGAUAACCGAAAGU (SEQ ID | MIMAT0000143 |
| | NO: 454) | |
| mmu-miR-99b*-4395307 | CAAGCUCGUGUCUGUGGGUCCG (SEQ ID | MIMAT0004525 |
| | NO: 455) | |
| rno-miR-28*-4395557 | CACUAGAUUGUGAGCUCCUGGA (SEQ ID | MIMAT0004716 |
| | NO: 456) | |
| rno-miR-463-4395751 | UGAUAGACGCCAAUUUGGGUAG (SEQ ID | MIMAT0005317 |
| | NO: 457) | |
| rno-miR-99a*-4395774 | CAAGCUCGUUUCUAUGGGUCUG (SEQ ID | MIMAT0004724 |
| | NO: 458) | |
| snoRNAl35-4380912 | CTAAAATAGCTGGAATTACCGGCAGATTGG | Mature miRNA |
| | TAGTGGTGAGCCTATGGTTTTCTGAAG | Control |
| | (SEQ ID NO: 459) | |

Table 9 depicts results from analysis of miRNAs from human PC RNA preparations.

TABLE 9
Exemplary miRNA sequences in serum starved human PCs

| | | Alternative |
|---|---|---|
| miRNA | Exemplary Sequence(s)(5′ to 3′) | Description |
| has-miR-155-4395459 | UUAAUGCUAAUCGUGAUAGGGGU | MIMAT0000646 |
| | (SEQ ID NO: 460) | |
| hsa-let-7a-4373169 | UGAGGUAGUAGGUUGUAUAGUU (SEQ ID | MIMAT0000062 |
| | NO: 461) | |
| hsa-let-7d-4395394 | AGAGGUAGUAGGUUGCAUAGUU (SEQ ID | MIMAT0000065 |
| | NO: 462) | |
| hsa-let-7f-4373164 | UGAGGUAGUAGAUUGUAUAGUU (SEQ ID | MIMAT0000067 |
| | NO: 463) | |
| hsa-let-7g-4395393 | UGAGGUAGUAGUUUGUACAGUU (SEQ ID | MIMAT0000414 |
| | NO: 464) | |
| hsa-miR-100-4373160 | AACCCGUAGAUCCGAACUUGUG (SEQ ID | MIMAT0000098 |
| | NO: 465) | |
| hsa-miR-103-4373158 | AGCAGCAUUGUACAGGGCUAUGA (SEQ ID | MIMAT0000101 |
| | NO: 466) | |
| hsa-miR-106a-4395280 | AAAAGUGCUUACAGUGCAGGUAG (SEQ ID | MIMAT0000103 |
| | NO: 467) | |
| hsa-miR-106b-4373155 | UAAAGUGCUGACAGUGCAGAU (SEQ ID | MIMAT0000680 |
| | NO: 468) | |
| hsa-miR-125a-5p-4395309 | UCCCUGAGACCCUUUAACCUGUGA (SEQ | MIMAT0000443 |
| | ID NO: 469) | |
| hsa-miR-125b-4373148 | UCCCUGAGACCCUAACUUGUGA (SEQ ID | MIMAT0000423 |
| | NO: 470) | |
| hsa-miR-126-4395339 | UCGUACCGUGAGUAAUAAUGCG (SEQ ID | MIMAT0000445 |
| | NO: 471) | |
| hsa-miR-127-3p-4373147 | UCGGAUCCGUCUGAGCUUGGCU (SEQ ID | MIMAT0000446 |
| | NO: 472) | |
| hsa-miR-132-4373143 | UAACAGUCUACAGCCAUGGUCG (SEQ ID | MIMAT0000426 |
| | NO: 473) | |
| hsa-miR-134-4373299 | UGUGACUGGUUGACCAGAGGGG (SEQ ID | MIMAT0000447 |
| | NO: 474) | |
| hsa-miR-138-4395395 | AGCUGGUGUUGUGAAUCAGGCCG (SEQ ID | MIMAT0000430 |
| | NO: 475) | |
| hsa-miR-139-5p-4395400 | UCUACAGUGCACGUGUCUCCAG (SEQ ID | MIMAT0000250 |
| | NO: 476) | |
| hsa-miR-140-3p-4395345 | UACCACAGGGUAGAACCACGG (SEQ ID | MIMAT0004597 |
| | NO: 477) | |
| hsa-miR-140-5p-4373374 | CAGUGGUUUUACCCUAUGGUAG (SEQ ID | MIMAT0000431 |
| | NO: 478) | |
| hsa-miR-143-4395360 | UGAGAUGAAGCACUGUAGCUC (SEQ ID | MIMAT0000435 |
| | NO: 479) | |
| hsa-miR-145-4395389 | GUCCAGUUUUCCCAGGAAUCCCU (SEQ ID | MIMAT0000437 |
| | NO: 480) | |
| hsa-miR-146b-5p-4373178 | UGAGAACUGAAUUCCAUAGGCU (SEQ ID | MIMAT0002809 |
| | NO: 481) | |
| hsa-miR-149-4395366 | UCUGGCUCCGUGUCUUCACUCCC (SEQ ID | MIMAT0000450 |
| | NO: 482) | |
| hsa-miR-15b-4373122 | UAGCAGCACAUCAUGGUUUACA (SEQ ID | MIMAT0000417 |
| | NO: 483) | |
| hsa-miR-17-4395419 | CAAAGUGCUUACAGUGCAGGUAG (SEQ ID | MIMAT0000070 |
| | NO: 484) | |
| hsa-miR-181a-4373117 | AACAUUCAACGCUGUCGGUGAGU (SEQ ID | MIMAT0000256 |
| | NO: 485) | |
| hsa-miR-186-4395396 | CAAAGAAUUCUCCUUUUGGGCU (SEQ ID | MIMAT0000456 |
| | NO: 486) | |
| hsa-miR-191-4395410 | CAACGGAAUCCCAAAAGCAGCUG (SEQ ID | MIMAT0000440 |
| | NO: 487) | |
| hsa-miR-193a-5p-4395392 | UGGGUCUUUGCGGGCGAGAUGA (SEQ ID | MIMAT0004614 |
| | NO: 488) | |
| hsa-miR-193b-4395478 | AACUGGCCCUCAAAGUCCCGCU (SEQ ID | MIMAT0002819 |
| | NO: 489) | |
| hsa-miR-195-4373105 | UAGCAGCACAGAAAUAUUGGC (SEQ ID | MIMAT0000461 |
| | NO: 490) | |
| hsa-miR-197-4373102 | UUCACCACCUUCUCCACCCAGC (SEQ ID | MIMAT0000227 |
| | NO: 491) | |
| hsa-miR-199a-3p-4395415 | ACAGUAGUCUGCACAUUGGUUA (SEQ ID | MIMAT0000232 |
| | NO: 492) | |
| hsa-miR-19a-4373099 | UGUGCAAAUCUAUGCAAAACUGA (SEQ ID | MIMAT0000073 |
| | NO: 493) | |
| hsa-miR-19b-4373098 | UGUGCAAAUCCAUGCAAAACUGA (SEQ ID | MIMAT0000074 |
| | NO: 494) | |
| hsa-miR-204-4373094 | UUCCCUUUGUCAUCCUAUGCCU (SEQ ID | MIMAT0000265 |
| | NO: 495) | |
| hsa-miR-20a-4373286 | UAAAGUGCUUAUAGUGCAGGUAG (SEQ ID | MIMAT0000075 |
| | NO: 496) | |
| hsa-miR-210-4373089 | CUGUGCGUGUGACAGCGGCUGA (SEQ ID | MIMAT0000267 |
| | NO: 497) | |
| hsa-miR-21-4373090 | UAGCUUAUCAGACUGAUGUUGA (SEQ ID | MIMAT0000076 |
| | NO: 498) | |
| hsa-miR-214-4395417 | ACAGCAGGCACAGACAGGCAGU (SEQ ID | MIMAT0000271 |
| | NO: 499) | |
| hsa-miR-221-4373077 | AGCUACAUUGUCUGCUGGGUUUC (SEQ ID | MIMAT0000278 |
| | NO: 500) | |
| hsa-miR-222-4395387 | AGCUACAUCUGGCUACUGGGU (SEQ ID | MIMAT0000279 |
| | NO: 501) | |
| hsa-miR-24-4373072 | UGGCUCAGUUCAGCAGGAACAG (SEQ ID | MIMAT0000080 |
| | NO: 502) | |
| hsa-miR-26a-4395166 | UUCAAGUAAUCCAGGAUAGGCU (SEQ ID | MIMAT0000082 |
| | NO: 503) | |
| hsa-miR-26b-4395167 | UUCAAGUAAUUCAGGAUAGGU (SEQ ID | MIMAT0000083 |
| | NO: 504) | |
| hsa-miR-27a-4373287 | UUCACAGUGGCUAAGUUCCGC (SEQ ID | MIMAT0000084 |
| | NO: 505) | |
| hsa-miR-27b-4373068 | UUCACAGUGGCUAAGUUCUGC (SEQ ID | MIMAT0000419 |
| | NO: 506) | |
| hsa-miR-28-3p-4395557 | CACUAGAUUGUGAGCUCCUGGA (SEQ ID | MIMAT0004502 |
| | NO: 507) | |
| hsa-miR-28-5p-4373067 | AAGGAGCUCACAGUCUAUUGAG (SEQ ID | MIMAT0000085 |
| | NO: 508) | |
| hsa-miR-29a-4395223 | UAGCACCAUCUGAAAUCGGUUA (SEQ ID | MIMAT0000086 |
| | NO: 509) | |
| hsa-miR-29c-4395171 | UAGCACCAUUUGAAAUCGGUUA (SEQ ID | MIMAT0000681 |
| | NO: 510) | |
| hsa-miR-30b-4373290 | UGUAAACAUCCUACACUCAGCU (SEQ ID | MIMAT0000420 |
| | NO: 511) | |
| hsa-miR-30c-4373060 | UGUAAACAUCCUACACUCUCAGC (SEQ ID | MIMAT0000244 |
| | NO: 512) | |
| hsa-miR-31-4395390 | AGGCAAGAUGCUGGCAUAGCU (SEQ ID | MIMAT0000089 |
| | NO: 513) | |
| hsa-miR-320-4395388 | AAAAGCUGGGUUGAGAGGGCGA | MIMAT0000510 |
| | (SEQ ID NO: 514) | |
| hsa-miR-323-3p-4395338 | CACAUUACACGGUCGACCUCU (SEQ ID | MIMAT0000755 |
| | NO: 515) | |
| hsa-miR-324-3p-4395272 | ACUGCCCCAGGUGCUGCUGG (SEQ ID | MIMAT0000762 |
| | NO: 516) | |
| hsa-miR-328-4373049 | CUGGCCCUCUCUGCCCUUCCGU (SEQ ID | MIMAT0000752 |
| | NO: 517) | |
| hsa-miR-331-3p-4373046 | GCCCCUGGGCCUAUCCUAGAA (SEQ ID | MIMAT0000760 |
| | NO: 518) | |
| hsa-miR-339-3p-4395295 | UGAGCGCCUCGACGACAGAGCCG (SEQ ID | MIMAT0004702 |
| | NO: 519) | |
| hsa-miR-342-3p-4395371 | UCUCACACAGAAAUCGCACCCGU (SEQ ID | MIMAT0000753 |
| | NO: 520) | |
| hsa-miR-345-4395297 | GCUGACUCCUAGUCCAGGGCUC (SEQ ID | MIMAT0000772 |
| | NO: 521) | |
| hsa-miR-34a-4395168 | UGGCAGUGUCUUAGCUGGUUGU (SEQ ID | MIMAT0000255 |
| | NO: 522) | |
| hsa-miR-362-5p-4378092 | AAUCCUUGGAACCUAGGUGUGAGU (SEQ | MIMAT0000705 |
| | ID NO: 523) | |
| hsa-miR-365-4373194 | UAAUGCCCCUAAAAAUCCUUAU (SEQ ID | MIMAT0000710 |
| | NO: 524) | |
| hsa-miR-374a-4373028 | UUAUAAUACAACCUGAUAAGUG (SEQ ID | MIMAT0000727 |
| | NO: 525) | |
| hsa-miR-374b-4381045 | AUAUAAUACAACCUGCUAAGUG (SEQ ID | MIMAT0004955 |
| | NO: 526) | |
| hsa-miR-376a-4373026 | AUCAUAGAGGAAAAUCCACGU | MIMAT0000729 |
| | (SEQ ID NO: 527) | |
| hsa-miR-376c-4395233 | AACAUAGAGGAAAUUCCACGU | MIMAT0000720 |
| | (SEQ ID NO: 528) | |
| hsa-miR-411-4381013 | UAGUAGACCGUAUAGCGUACG (SEQ ID | MIMAT0003329 |
| | NO: 529) | |
| hsa-miR-425-4380926 | AAUGACACGAUCACUCCCGUUGA (SEQ ID | MIMAT0003393 |
| | NO: 530) | |
| hsa-miR-431-4395173 | UGUCUUGCAGGCCGUCAUGCA (SEQ ID | MIMAT0001625 |
| | NO: 531) | |
| hsa-miR-452-4395440 | AACUGUUUGCAGAGGAAACUGA (SEQ ID | MIMAT0001635 |
| | NO: 532) | |
| hsa-miR-454-4395434 | UAGUGCAAUAUUGCUUAUAGGGU (SEQ ID | MIMAT0003885 |
| | NO: 533) | |
| hsa-miR-483-5p-4395449 | AAGACGGGAGGAAAGAAGGGAG (SEQ ID | MIMAT0004761 |
| | NO: 534) | |
| hsa-miR-484-4381032 | UCAGGCUCAGUCCCCUCCCGAU (SEQ ID | MIMAT0002174 |
| | NO: 535) | |
| hsa-miR-485-3p-4378095 | GUCAUACACGGCUCUCCUCUCU (SEQ ID | MIMAT0002176 |
| | NO: 536) | |
| hsa-miR-486-5p-4378096 | UCCUGUACUGAGCUGCCCCGAG (SEQ ID | MIMAT0002177 |
| | NO: 537) | |
| hsa-miR-491-5p-4381053 | AGUGGGGAACCCUUCCAUGAGG (SEQ ID | MIMAT0002807 |
| | NO: 538) | |
| hsa-miR-495-4381078 | AAACAAACAUGGUGCACUUCUU (SEQ ID | MIMAT0002817 |
| | NO: 539) | |
| hsa-miR-532-3p-4395466 | CCUCCCACACCCAAGGCUUGCA (SEQ ID | MIMAT0004780 |
| | NO: 540) | |
| hsa-miR-532-5p-4380928 | CAUGCCUUGAGUGUAGGACCGU (SEQ ID | MIMAT0002888 |
| | NO: 541) | |
| hsa-miR-539-4378103 | GGAGAAAUUAUCCUUGGUGUGU (SEQ ID | MIMAT0003163 |
| | NO: 542) | |
| hsa-miR-574-3p-4395460 | CACGCUCAUGCACACACCCACA (SEQ ID | MIMAT0003239 |
| | NO: 543) | |
| hsa-miR-590-5p-4395176 | GAGCUUAUUCAUAAAAGUGCAG (SEQ ID | MIMAT0003258 |
| | NO: 544) | |
| hsa-miR-628-5p-4395544 | AUGCUGACAUAUUUACUAGAGG (SEQ ID | MIMAT0004809 |
| | NO: 545) | |
| hsa-miR-671-3p-4395433 | UCCGGUUCUCAGGGCUCCACC (SEQ ID | MIMAT0004819 |
| | NO: 546) | |
| hsa-miR-708-4395452 | AAGGAGCUUACAAUCUAGCUGGG (SEQ ID | MIMAT0004926 |
| | NO: 547) | |
| hsa-miR-744-4395435 | UGCGGGGCUAGGGCUAACAGCA (SEQ ID | MIMAT0004945 |
| | NO: 548) | |
| hsa-miR-886-3p-4395305 | CGCGGGUGCUUACUGACCCUU | MIMAT0004905 |
| | (SEQ ID NO: 549) | |
| hsa-miR-886-5p-4395304 | CGGGUCGGAGUUAGCUCAAGCGG | MIMAT0004905 |
| | (SEQ ID NO: 550) | |
| hsa-miR-92a-4395169 | UAUUGCACUUGUCCCGGCCUGU (SEQ ID | MIMAT0000092 |
| | NO: 551) | |
| hsa-miR-93-4373302 | CAAAGUGCUGUUCGUGCAGGUAG (SEQ ID | MIMAT0000093 |
| | NO: 552) | |
| hsa-miR-99a-4373008 | AACCCGUAGAUCCGAUCUUGUG (SEQ ID | MIMAT0000097 |
| | NO: 553) | |
| RNU48-4373383 | GATGACCCCAGGTAACTCTGAGTGTGTCGC | Mature miRNA |
| | TGATGCCATCACCGCAGCGCTCTGACC | Control |
| | (SEQ ID NO: 554) | |
| hsa-let-7b#-002404 | UGAGGUAGUAGGUUGUGUGGUU | MIMAT0000063 |
| | (SEQ ID NO: 555) | |
| hsa-miR-10b#-002315 | UACCCUGUAGAACCGAAUUUGUG | MIMAT0000254 |
| | (SEQ ID NO: 556) | |
| hsa-miR-1180-002847 | UUUCCGGCUCGCGUGGGUGUGU (SEQ ID | MIMAT0005825 |
| | NO: 557) | |
| hsa-miR-1201-002781 | AGCCUGAUUAAACACAUGCUCUGA (SEQ | |
| | ID NO: 558) | |
| hsa-miR-1208-002880 | UCACUGUUCAGACAGGCGGA (SEQ ID | MIMAT0005873 |
| | NO: 559) | |
| hsa-miR-1233-002768 | UGAGCCCUGUCCUCCCGCAG (SEQ ID | MIMAT0005588 |
| | NO: 560) | |
| hsa-miR-1247-002893 | ACCCGUCCCGUUCGUCCCCGGA (SEQ ID | MIMAT0005899 |
| | NO: 561) | |
| hsa-miR-125b-1#-002378 | ACGGGUUAGGCUCUUGGGAGCU | MIMAT0004592 |
| | (SEQ ID NO: 562) | |
| hsa-miR-1260-002896 | AUCCCACCUCUGCCACCA (SEQ ID NO: 563) | MIMAT0005911 |
| hsa-miR-1271-002779 | CUUGGCACCUAGCAAGCACUCA (SEQ ID | MIMAT0005796 |
| | NO: 564) | |
| hsa-miR-1274A-002883 | GUCCCUGUUCAGGCGCCA | |
| | (SEQ ID NO: 565) | |
| hsa-miR-1274B-002884 | UCCCUGUUCGGGCGCCA (SEQ ID NO: 566) | MIMAT0005938 |
| hsa-miR-1275-002840 | GUGGGGGAGAGGCUGUC (SEQ ID NO: 567) | MIMAT0005929 |
| hsa-miR-1290-002863 | UGGAUUUUUGGAUCAGGGA (SEQ ID | MIMAT0005880 |
| | NO: 568) | |
| hsa-miR-130b#-002114 | CAGUGCAAUGAUGAAAGGGCAU | MIMAT0000691 |
| | (SEQ ID NO: 569) | |
| hsa-miR-145#-002149 | GUCCAGUUUUCCCAGGAAUCCCU | MIMAT0000437 |
| | (SEQ ID NO: 570) | |
| hsa-miR-149#-002164 | UCUGGCUCCGUGUCUUCACUCCC | MIMAT0000450 |
| | (SEQ ID NO: 571) | |
| hsa-miR-151-3p-002254 | CUAGACUGAAGCUCCUUGAGG (SEQ ID | MIMAT0000757 |
| | NO: 572) | |
| hsa-miR-151-5P-002642 | UCGAGGAGCUCACAGUCUAGU (SEQ ID | MIMAT0004697 |
| | NO: 573) | |
| hsa-miR-181a-2#-002317 | ACCACUGACCGUUGACUGUACC | MIMAT0004558 |
| | (SEQ ID NO: 574) | |
| hsa-miR-193b#-002366 | AACUGGCCCUCAAAGUCCCGCU | MIMAT0002819 |
| | (SEQ ID NO: 575) | |
| hsa-miR-213-000516 | ACCAUCGACCGUUGAUUGUACC | MIMAT0000270 |
| | (SEQ ID NO: 576) | |
| hsa-miR-214#-002293 | ACAGCAGGCACAGACAGGCAGU | MIMAT0000271 |
| | (SEQ ID NO: 577) | |
| hsa-miR-22#-002301 | AAGCUGCCAGUUGAAGAACUGU | MIMAT0000077 |
| | (SEQ ID NO: 578) | |
| hsa-miR-24-2#-002441 | UGCCUACUGAGCUGAAACACAG | MIMAT0004497 |
| | (SEQ ID NO: 579) | |
| hsa-miR-26a-1#-002443 | CCUAUUCUUGGUUACUUGCACG | MIMAT0004499 |
| | (SEQ ID NO: 580) | |
| hsa-miR-27a#-002445 | UUCACAGUGGCUAAGUUCCGC | MIMAT0000084 |
| | (SEQ ID NO: 581) | |
| hsa-miR-27b#-002174 | UUCACAGUGGCUAAGUUCUGC | MIMAT0000419 |
| | (SEQ ID NO: 582) | |
| hsa-miR-30a-3p-000416 | CUUUCAGUCGGAUGUUUGCAGC | MIMAT0000088 |
| | (SEQ ID NO: 583) | |
| hsa-miR-30a-5p-000417 | UGUAAACAUCCUCGACUGGAAG | MIMAT0000087 |
| | (SEQ ID NO: 584) | |
| hsa-miR-30e-3p-000422 | CUUUCAGUCGGAUGUUUACAGC | MIMAT0000693 |
| | (SEQ ID NO: 585) | |
| hsa-miR-31#-002113 | AGGCAAGAUGCUGGCAUAGCU | MIMAT0000089 |
| | (SEQ ID NO: 586) | |
| hsa-miR-335#-002185 | UCAAGAGCAAUAACGAAAAAUGU | MIMAT0002819 |
| | (SEQ ID NO: 587) | |
| hsa-miR-34b-002102 | CAAUCACUAACUCCACUGCCAU (SEQ ID | MIMAT0004676 |
| | NO: 588) | |
| hsa-miR-378-002243 | ACUGGACUUGGAGUCAGAAGG (SEQ ID | MIMAT0000732 |
| | NO: 589) | |
| hsa-miR-409-3p-002332 | GAAUGUUGCUCGGUGAACCCCU (SEQ ID | MIMAT0001639 |
| | NO: 590) | |
| hsa-miR-424#-002309 | CAGCAGCAAUUCAUGUUUUGAA | MIMAT0001341 |
| | (SEQ ID NO: 591) | |
| hsa-miR-425#-002302 | AAUGACACGAUCACUCCCGUUGA | MIMAT0003393 |
| | (SEQ ID NO: 592) | |
| hsa-miR-432-001026 | UCUUGGAGUAGGUCAUUGGGUGG (SEQ ID | MIMAT0002814 |
| | NO: 593) | |
| hsa-miR-520D-3P-002743 | AAAGUGCUUCUCUUUGGUGGGU (SEQ ID | MIMAT0002856 |
| | NO: 594) | |
| hsa-miR-543-002376 | AAACAUUCGCGGUGCACUUCUU (SEQ ID | MIMAT0004954 |
| | NO: 595) | |
| hsa-miR-545#-002266 | UCAGCAAACAUUUAUUGUGUGC | MIMAT0003165 |
| | (SEQ ID NO: 596) | |
| hsa-miR-596-001550 | AAGCCUGCCCGGCUCCUCGGG (SEQ ID | MIMAT0003264 |
| | NO: 597) | |
| hsa-miR-625#-002432 | AGGGGGAAAGUUCUAUAGUCC | MIMAT0003294 |
| | (SEQ ID NO: 598) | |
| hsa-miR-641-001585 | AAAGACAUAGGAUAGAGUCACCUC (SEQ | MIMAT0003311 |
| | ID NO: 599) | |
| hsa-miR-664-002897 | UAUUCAUUUAUCCCCAGCCUACA | MIMAT0005949 |
| | (SEQ ID NO: 600) | |
| hsa-miR-720-002895 | UCUCGCUGGGGCCUCCA (SEQ ID NO: 601) | MIMAT0005954 |
| hsa-miR-766-001986 | ACUCCAGCCCCACAGCCUCAGC (SEQ ID | MIMAT0003888 |
| | NO: 332) | |
| hsa-miR-767-3p-001995 | UCUGCUCAUACCCCAUGGUUUCU (SEQ ID | MIMAT0003883 |
| | NO: 602) | |
| hsa-miR-769-5p-001998 | UGAGACCUCUGGGUUCUGAGCU (SEQ ID | MIMAT0003886 |
| | NO: 603) | |
| hsa-miR-875-5p-002203 | UAUACCUCAGUUUUAUCAGGUG (SEQ ID | MIMAT0004922 |
| | NO: 604) | |
| hsa-miR-93#-002139 | CAAAGUGCUGUUCGUGCAGGUAG | MIMAT0000093 |
| | (SEQ ID NO: 605) | |
| rno-miR-7#-001338 | CAACAAAUCACAGUCUGCCAUA | MIMAT0004553 |
| | (SEQ ID NO: 606) | |
| RNU44-001094 | CCTGGATGATGATAGCAAATGCTGACTGAA | Mature miRNA |
| | CATGAAGGTCTTAATTAGCTCTAACTGACT | Control |
| | (SEQ ID NO: 607) | |
| RNU48-001006 | GATGACCCCAGGTAACTCTGAGTGTGTCGC | Mature miRNA |
| | TGATGCCATCACCGCAGCGCTCTGACC | Control |
| | (SEQ ID NO: 608) | |

Table 10 depicts miRNAs in common between human and rat PCs grown under serum starvation conditions. As can be seen in Table 10, 22 miRNA sequences were found in common between human and rat PCs grown under serum starvation conditions.

TABLE 10
miRNAs found in both serum starved rat and human PCs

| | Exemplary | | | Exemplary | |
|---|---|---|---|---|---|
| miRNA in | Sequence(s) | Alternative | miRNA in | Sequence(s) | Alternative |
| Rat PCs | (5′ to 3′) | Description | Human PCs | (5′ to 3′) | Description |
| mmu-miR- | UCCCUGAG | MIMAT0000135 | hsa-miR-125a- | UCCCUGAG | MIMAT0000443 |
| 125a-5p- | ACCCUUUA | | 5p-4395309 | ACCCUUUA | |
| 4395309 | ACCUGUGA | | | ACCUGUGA | |
| | (SEQ ID | | | (SEQ ID | |
| | NO: 324) | | | NO: 469) | |
| mmu-miR- | ACGGGUUA | MIMAT0004669 | hsa-miR-125b- | UCCCUGAG | MIMAT0000423 |
| 125b-3p- | GGCUCUUG | | 4373148 | ACCCUAAC | |
| 4395489 | GGAGCU | | | UUGUGA | |
| | (SEQ ID | | | (SEQ ID | |
| | NO: 325) | | | NO: 470) | |
| mmu-miR- | AGCUGGUG | MIMAT0000150 | hsa-miR-138- | AGCUGGUG | MIMAT0000430 |
| 138-4395395 | UUGUGAAU | | 4395395 | UUGUGAAU | |
| | CAGGCCG | | | CAGGCCG | |
| | (SEQ ID | | | (SEQ ID | |
| | NO: 328) | MIMAT0000247 | hsa-miR-143- | NO: 475) | MIMAT0000435 |
| mmu-miR- | UGAGAUGA | | 4395360 | UGAGAUGA | |
| 143-4395360 | AGCACUGU | | | AGCACUGU | |
| | AGCUC (SEQ | | | AGCUC (SEQ | |
| | ID NO: 341) | | | ID NO: 479) | |
| mmu-miR- | UUCACCAC | | hsa-miR-197- | UUCACCAC | MIMAT0000227 |
| 197-4373102 | CUUCUCCA | | 4373102 | CUUCUCCA | |
| | CCCAGC | | | CCCAGC | |
| | (SEQ ID | | | (SEQ ID | |
| | NO: 353) | | | NO: 491) | |
| mmu-miR- | ACAGUAGU | MIMAT0000230 | hsa-miR-199a- | ACAGUAGU | MIMAT0000232 |
| 199a-3p- | CUGCACAU | | 3p-4395415 | CUGCACAU | |
| 4395415 | UGGUUA | | | UGGUUA | |
| | (SEQ ID | | | (SEQ ID | |
| | NO: 354) | | | NO: 492) | |
| mmu-miR- | CUGUGCGU | MIMAT0000658 | hsa-miR-210- | CUGUGCGU | MIMAT0000267 |
| 210-4373089 | GUGACAGC | | 4373089 | GUGACAGC | |
| | GGCUGA | | | GGCUGA | |
| | (SEQ ID | | | (SEQ ID | |
| | NO: 357) | | | NO: 497) | |
| mmu-miR-21- | UAGCUUAU | MIMAT0000530 | hsa-miR-21- | UAGCUUAU | MIMAT0000076 |
| 4373090 | CAGACUGA | | 4373090 | CAGACUGA | |
| | UGUUGA | | | UGUUGA | |
| | (SEQ ID | | | (SEQ ID | |
| | NO: 358) | | | NO: 498) | |
| mmu-miR- | AGCUACAU | MIMAT0000670 | hsa-miR-222- | AGCUACAU | MIMAT0000279 |
| 222-4395387 | CUGGCUAC | | 4395387 | CUGGCUAC | |
| | UGGGU | | | UGGGU | |
| | (SEQ ID | | | (SEQ ID | |
| | NO: 359) | | | NO: 501) | |
| mmu-miR- | UUCAAGUA | MIMAT0000533 | hsa-miR-26a- | UUCAAGUA | MIMAT0000082 |
| 26a-4395166 | AUCCAGGA | | 4395166 | AUCCAGGA | |
| | UAGGCU | | | UAGGCU | |
| | (SEQ ID | | | (SEQ ID | |
| | NO: 362) | | | NO: 503) | |
| mmu-miR- | UAGCACCA | MIMAT0000536 | hsa-miR-29c- | UAGCACCA | MIMAT0000681 |
| 29c-4395171 | UUUGAAAU | | 4395171 | UUUGAAAU | |
| | CGGUUA | | | CGGUUA | |
| | (SEQ ID | | | (SEQ ID | |
| | NO: 364) | | | NO: 510) | |
| mmu-miR- | AAAAGCUG | MIMAT0000666 | hsa-miR-320- | AAAAGCUG | MIMAT0000510 |
| 320-4395388 | GGUUGAGA | | 4395388 | GGUUGAGA | |
| | GGGCGA | | | GGGCGA | |
| | (SEQ ID | | | (SEQ ID | |
| | NO: 365) | | | NO: 514) | |
| mmu-miR- | UGCGGGGC | MIMAT0004187 | hsa-miR-744- | UGCGGGGC | MIMAT0004945 |
| 744-4395435 | UAGGGCUA | | 4395435 | UAGGGCUA | |
| | ACAGCA | | | ACAGCA | |
| | (SEQ ID | | | (SEQ ID | |
| | NO: 390) | | | NO: 548) | |
| mmu-miR- | CAGAUUCG | MIMAT0004538 | hsa-miR-10b#- | UACCCUGU | MIMAT0000254 |
| 10b*-4395702 | AUUCUAGG | | 002315 | AGAACCGA | |
| | GGAAUA | | | AUUUGUG | |
| | (SEQ ID | | | (SEQ ID | |
| | NO: 408) | | | NO: 556) | |
| mmu-miR- | ACUCUUUC | MIMAT0004583 | hsa-miR- | CAGUGCAA | MIMAT0000691 |
| 130b*- | CCUGUUGC | | 130b#-002114 | UGAUGAAA | |
| 4395590 | ACUACU | | | GGGCAU | |
| | (SEQ ID | | | (SEQ ID | |
| | NO: 409) | | | NO: 569) | |
| | UCUGGCUC | | | UCUGGCUC | |
| mmu-miR- | CGUGUCUU | MIMAT0000159 | hsa-miR-149#- | CGUGUCUU | MIMAT0000450 |
| 149-4395366 | CACUCCC | | 002164 | CACUCCC | |
| | (SEQ ID | | | (SEQ ID | |
| | NO: 411) | | | NO: 571) | |
| mmu-miR- | UGCCUGUC | MIMAT0004664 | hsa-miR-214#- | ACAGCAGG | MIMAT0000271 |
| 214*-4395404 | UACACUUG | | 002293 | CACAGACA | |
| | CUGUGC | | | GGCAGU | |
| | (SEQ ID | | | (SEQ ID | |
| | NO: 419) | | | NO: 577) | |
| mmu-miR- | CUUUCAGU | MIMAT0000129 | hsa-miR-30a- | CUUUCAGU | MIMAT0000088 |
| 30a*-4373062 | CGGAUGUU | | 3p-000416 | CGGAUGUU | |
| | UGCAGC | | | UGCAGC | |
| | (SEQ ID | | | (SEQ ID | |
| | NO: 428) | | | NO: 583) | |
| mmu-miR- | CUUUCAGU | MIMAT0000249 | hsa-miR-30e- | CUUUCAGU | MIMAT0000693 |
| 30e*-4373057 | CGGAUGUU | | 3p-000422 | CGGAUGUU | |
| | UACAGC | | | UACAGC | |
| | (SEQ ID | | | (SEQ ID | |
| | NO: 431) | | | NO: 585) | |
| mmu-miR- | ACUGGACU | MIMAT0003151 | hsa-miR-378- | ACUGGACU | MIMAT0000732 |
| 378-4395354 | UGGAGUCA | | 002243 | UGGAGUCA | |
| | GAAGG | | | GAAGG | |
| | (SEQ ID | | | (SEQ ID | |
| | NO: 436) | | | NO: 589) | |
| mmu-miR- | AUCUCGCU | MIMAT0003484 | hsa-miR-720- | UCUCGCUG | MIMAT0005954 |
| 720-4381052 | GGGGCCUC | | 002895 | GGGCCUCC | |
| | CA (SEQ ID | | | A (SEQ ID | |
| | NO: 447) | | | NO: 601) | |
| mmu-miR- | UAUACCUC | MIMAT0004937 | hsa-miR-875- | UAUACCUC | MIMAT0004922 |
| 8′75-5p- | AGUUUUAU | | 5p-002203 | AGUUUUAU | |
| 4395314 | CAGGUG | | | CAGGUG | |
| | (SEQ ID | | | (SEQ ID | |
| | NO: 452) | | | NO: 604) | |

### EQUIVALENTS AND SCOPE

Those skilled in the art will recognize, or be able to ascertain using no more than routine experimentation, many equivalents to the specific embodiments, described herein. The scope of the present invention is not intended to be limited to the above Description, but rather is as set forth in the appended claims.

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any cell type; any neuronal cell system; any reporter of synaptic vesicle cycling; any electrical stimulation system; any imaging system; any synaptic vesicle cycling assay; any synaptic vesicle cycle modulator; any method of use; etc.) can be excluded from any one or more claims, for any reason, whether or not related to the existence of prior art.

### INCORPORATION OF REFERENCES

All publications and patent documents cited in this application are incorporated by reference in their entirety to the same extent as though the contents of each individual publication or patent document were incorporated herein.

1. A method for treating acute tissue damage comprising a step of:
administering a population of cells to an individual suffering from a disease, disorder or condition characterized by acute damage to one or more tissues, wherein the cells are originated from an adult tissue and wherein the cells induce tissue repair, regeneration, remodeling, reconstitution or differentiation.

2. The method of claim 1, wherein the one or more tissues are selected from the group consisting of kidney, heart, liver, lungs, pancreas, brain, intestine, bones, tendons, cornea, skin, muscle, veins, spinal cord, spleen, blood, and combinations thereof.

3. The method of claim 1, wherein the one or more tissues are kidney and/or heart.

4. The method of claim 1, wherein the acute damage is ischemic damage.

5. The method of claim 1, wherein the acute damage is associated with tissue transplantation.

6. The method of claim 1, wherein the acute damage is associated with radiation or chemical injury or therapy.

7. The method of claim 1, wherein the disease, disorder or condition is selected from the group consisting of myocardial infarct, acute renal failure, type I diabetes, and combinations thereof.

8. The method of claim 1, wherein the cells are originated from an adult tissue that is distinct from the damaged tissue.

9. The method of claim 1, wherein the cells are originated from an adult tissue that is from a different species.

10. The method of claim 1, wherein the adult tissue is a human adult tissue.

11. The method of, wherein the adult tissue is a non-human adult tissue.

12. The method of claim 1, wherein the adult tissue is selected from the group consisting of pancreas, kidney, breast, lymph node, liver, spleen, myometrium, peripheral blood, cord blood, and bone marrow, and combination thereof.

13. The method of claim 1, wherein the cells are first cultivated in a cell culture medium under conditions and time sufficient for cell proliferation.

14. The method of claim 13, wherein the cell culture medium is a Matrigel free culture medium comprising serum.

15. The method of claim 13, wherein the cells are first treated to reduce a telomeric attrition rate before the cultivating step.

16. The method of claim 1, wherein the population of cells are substantially homogenous.

17. The method of any one of the preceding claims, wherein at least 50% of the population of cells express one or more markers selected from the group consisting of CD24, c-myc, HLA class 1 ABC, ICAM3, Nestin, Nanog, Oct4, Integrin α2+b1, Ngn3, and CD130.

18.-19. (canceled)

20. The method of claim 17, wherein the one or more markers comprise Oct4, Nanog, and c-myc.

21. The method of claim 1, wherein at least 50% of the population of cells express Nestin.

22. The method of claim 1, wherein the cells do not express at least one marker selected from the group consisting of CD34, CD105, VCAM1, CXCR2, CD44, CD73, ICAM1, and NCAM.

23.-73. (canceled)
